(19) United States
(12) Patent Application Publication
(10) Pub. No.: US 2022/0025045 A1
(43) Pub. Date: Jan. 27, 2022

(54) COMPOUNDS AND METHODS FOR TREATMENT OF HEAD AND NECK CANCER

(71) Applicant: INNATE PHARMA, MARSEILLE (FR)

(72) Inventors: OLIVIER BENAC, MARSEILLE (FR); STÉPHANIE CHANTEUX, MARSEILLE (FR); IVAN PERROT, CASSIS (FR); BENJAMIN ROSSI, MARSEILLE (FR); NICOLAS VIAUD, AUBAGNE (FR)

(21) Appl. No.: 17/418,271

(22) PCT Filed: Dec. 20, 2019
(86) PCT No.: PCT/EP2019/086861

§ 371 Date: Jun. 25, 2021

Related U.S. Application Data
(60) Provisional application No. 62/784,862, filed on Dec. 26, 2018.

(51) Int. Cl. C07K 16/28; A61P 35/00

(52) U.S. Cl. CPC C07K 16/2803; C07K 16/2863; A61P 35/00; C07K 2317/76; C07K 2317/24; C07K 2317/565; A61K 2039/507; C07K 2317/41; C07K 2317/52; C07K 2317/73; C07K 2317/71; C07K 2317/21; C07K 2317/56

(57) ABSTRACT

This invention relates to the use of ILT-2-targeting agents for the treatment of cancers head and neck cancers. This invention also provides advantageous combination regimens for use with ILT-2-targeting agents for the treatment of cancers.

### CROSS-REFERENCE TO RELATED APPLICATIONS

This application claims the benefit of U.S. Provisional Application No. 62/784,862 filed 26 Dec. 2018; which is incorporated herein by reference in its entirety; including any drawings.

### REFERENCE TO SEQUENCE LISTING

The present application is being filed along with a Sequence Listing in electronic format. The Sequence Listing is provided as a file entitled “LILRB1-HN_ST25”, created 20 December 2019, which is 184 KB in size. The information in the electronic format of the Sequence Listing is incorporated herein by reference in its entirety.

### FIELD OF THE INVENTION

This invention relates to the use of ILT-2-targeting agents for the treatment of cancers head and neck cancers. This invention also provides advantageous combination regimens for use with ILT-2-targeting agents for the treatment of cancers.

### BACKGROUND OF THE INVENTION

Ig-like transcripts (ILTs), also called lymphocyte inhibitory receptors or leukocyte immunoglobulin- (Ig-) like receptors (LIR/LILRs) that correspond to CD85. This family of proteins is encoded by more than 10 genes located in the 19q13.4 chromosome, and includes both activating and inhibitory members. Inhibitory LILRs transmit signals through their long cytoplasmic tails, which contain between two and four immunoreceptor tyrosine-based inhibitory domains (ITIMs) that, upon phosphorylation, recruit SHP-1 and SHP-2 phosphatases which mediate inhibition of various intracellular signal pathways. ILT-2 is a receptor for class I MHC antigens and recognizes a broad spectrum of HLA-A, HLA-B, HLA-C and HLA-G alleles. ILT-2 (LILRB1) is also a receptor for H301/UL18, a human cytomegalovirus class I MHC homolog. Ligand binding results in inhibitory signals and down-regulation of the immune response.

In addition to expression on dendritic cells (DCs), ILT2 proteins have also been reported to be expressed in NK cells. NK cells are mononuclear cell that develop in the bone marrow from lymphoid progenitors, and morphological features and biological properties typically include the expression of the cluster determinants (CDs) CD16, CD56, and/or CD57; the absence of the alpha/beta or gamma/delta TCR complex on the cell surface; the ability to bind to and kill target cells that fail to express “self” major histocompatibility complex (MHC)/human leukocyte antigen (HLA) proteins; and the ability to kill tumor cells or other diseased cells that express ligands for activating NK receptors. NK cells are characterized by their ability to bind and kill several types of tumor cell lines without the need for prior immunization or activation. NK cells can also release soluble proteins and cytokines that exert a regulatory effect on the immune system; and can undergo multiple rounds of cell division and produce daughter cells with similar biologic properties as the parent cell. Normal, healthy cells are protected from lysis by NK cells.

Based on their biological properties, various therapeutic strategies have been proposed in the art that rely on a modulation of NK cells. However, NK cell activity is regulated by a complex mechanism that involves both stimulating and inhibitory signals. Briefly, the lytic activity of NK cells is regulated by various cell surface receptors that transduce either positive or negative intracellular signals upon interaction with ligands on the target cell. The balance between positive and negative signals transmitted via these receptors determines whether or not a target cell is lysed (killed) by a NK cell. NK cell stimulatory signals can be mediated by Natural Cytotoxicity Receptors (NCR) such as NKp30, NKp44, and NKp46; as well as NKG2C receptors, NKG2D receptors, certain activating Killer Ig-like Receptors (KIRs), and other activating NK receptors (Lanier, Annual Review of Immunology 2005; 23:225-74).

Based on their biological properties, various strategies have been proposed in the art that rely on a modulation of ILT family members, notably vaccination strategies including inhibitors of ILT to relieve ILT-mediated tolerance in dendritic cells. The ILT family and its ligands are also of interest in view of reports correlating HLA-G with inhibition of immune cells such as NK cells. Wan et al. (Cell Physiol Biochem 2017; 44:1828-1841) reported that HLA-G, a natural ligand of several immune receptors including ILT2, ILT4 and KIR2DL4, can inhibit the function of many immune cells by binding to cell surface-expressed receptors.

The interactions of HLA class I molecules with ILT proteins is complex. HLA-G binds not only to ILT2 but also to ILT4 and other receptors (e.g. of the KIR family). Furthermore, many isoforms of HLA-G exist, and only the form HLA-G1 that associates with beta-2-microglobulin (and its soluble/secreted form HLA-G7) associate with bind to ILT2, whereas all forms HLA-G1, -G2, -G3, -G4, -G5, -G6 and -G7 associate with ILT4. Likewise, ILT2 and ILT4 bind not only HLA-G, but also to other MHC class I molecules. ILT2 and ILT4 use their two membrane distal domains (D1 and D2) to recognize the a3 domain and β2m subunit of MHC molecules, both of which are conserved among classical and non-classical MHC class I molecules. Kirwan and Burshtyn (J Immunol 2005; 175:5006-5015) reported that while ILT2 was found to have an inhibitory role on NK cell lines made to overexpress ILT2, the amount of ILT2 on normal (primary) NK cells is held below the threshold that would allow direct recognition of most MHC-I alleles. The authors consequently propose that in normal NK cells ILT2 is not active on its own but could cooperate with inhibitory KIR receptors to increase the functional range of KIRs' interaction with HLA-C molecules. More recently, Heidenreich et al. 2012 (Clinical and Developmental Immunology. Volume 2012, Article ID 652130)) concluded that ILT2 alone does not directly influence NK-cell-mediated cytotoxicity against myeloma.

Various groups have proposed to treat cancer by using antibodies or other agents that bind or target HLA-G, thereby removing the HLA-G-mediated immunosuppression and blocking of all the ILT and other receptors that interact with HLA-G such as ILT2, ILT4, KIR2DL4 and/or others (see, e.g., WO2018/091580). However, targeting HLA-G does not inhibit the interaction (if any) of ILT2 with other HLA class I ligands of ILT proteins. Despite the interest in ILT receptors related to the proposed role of HLA-G in tumor escape, there has been no clinical development of therapeutic agents that provide inhibition of ILT2.

Head and neck squamous cell carcinoma (HNSCC) has an incidence of −600,000 cases per year and mortality rate of −50%. The major risk factors for HNSCC are tobacco use, alcohol consumption, and infection with human papilloma virus (HPV). Despite advances in knowledge of its epidemiology and pathogenesis, the survival rates for many types of HNSCC have improved little over the past forty years. The overall 5-year survival rate of HNSCC patients is only about 50%. Tobacco, alcohol consumption and viral agents are the major risk factors for development of HNSCC. These risk factors, together with genetic susceptibility, result in the accumulation of multiple genetic and epigenetic alterations in a multi-step process of cancer development, and the understanding of such molecular carcinogenesis of HNSCC is being used for the development of targeted agents for treating HNSCC.

The idea of immunotherapy as a treatment for HNSCC has been in existence for decades, and attempts at treating HNSCC have involved targeting of tumor-specific antigens. Although improvements have been made in using such immune stimulatory treatment strategies for a variety of solid cancers, the use of these strategies for patients with head and neck squamous cell carcinoma (HNSCC) is lagging behind. Immunotherapeutic approaches for HNSCC are particularly complicated by the profound immune suppression that is induced by HNSCC, which potentially decreases the effectiveness of immune stimulatory efforts. A review of mechanisms by which HNSCC escapes the anti-tumor immune response, such as down-modulation of HLA class I, is provided in Duray et al. (2010) Clin. Dev. Immunol. Article ID 701657; 2010: 1-15.

The anti-EGFR monoclonal antibody cetuximab is thought to act through blocking oncogenic signaling of the EGF receptor pathway and by inducing Fcγ receptor-mediated antibody dependent cellular cytotoxicity (ADCC). In HNSCC however, ADCC may be affected by the profound immune suppression that is induced. At the same time, blocking oncogenic signaling of the EGF receptor pathway results in posttranscriptional regulation in tumor cells of major histocompatibility complex (MHC) class I-related antigens of the MICA/B and ULBP protein families which are recognized by the activating receptor NKG2D on NK cells and subsets of T cells. In particular, the expression by tumor cells of these stress-related antigens which are the natural ligands of NKG2D is decreased by clinical EGFR inhibitors, thus potentially decreasing the tumor cells' visibility to NK and T cells (Vantourout et al., Sci. Transl. Med. 6: 231ra49 (2014).

### SUMMARY OF THE INVENTION

Herein we studied neutralizing, non-FcγR-binding, specific anti-ILT2 antibodies that are able to induce an increase in the cytotoxic activity of primary NK cells from human donors toward tumor cells. When we studied HNSCC cells of different origins we found that unlike cells from other tumor types, they were negative for surface expression of HLA-A2 and HLA-G, the ligands of ILT2 that are believed to be important in mediating the inhibition of ILT2+ NK and T cells. However, we observed that the combined use of cetuximab and neutralizing anti-ILT2 antibodies resulted strong anti-tumor activity by human NK cells. The combination was particularly effective in causing NK cells to lyse cancer cells through ADCC. The results suggest that HNSCC cells express ligands of ILT2 other than HLA-A2 and HLA-G that are able to induce strong inhibition of cytotoxicity of NK cells, and that such inhibition can be overcome through the use of neutralizing anti-ILT2 antibodies.

The anti-ILT-2 antibodies used herein are examples of antibodies capable of inducing strong NK-mediated cytotoxic activity in primary human NK cells (e.g., donor derived NK cells) that have lower levels of expression of ILT2, and which bind to certain epitopes present solely on ILT2 (and not, e.g. on ILT-1, 4, -5 or -6). Without wishing to be bound by theory, binding ILT2 without binding to ILT6 may have the advantage of providing stronger potentiation of NK and/or CD8 T cell activity because ILT6 is naturally present as a soluble protein which binds HLA class I molecules, thereby acting as a natural inhibitor of inhibitory receptors (other than ILT2) on the surface of the NK and/or T cells. The anti-ILT-2 antibodies used were modified to reduce and/or eliminate binding to human Fcγ receptors.

Provided herein is a combination treatment comprising an antibody that binds EGFR, e.g. cetuximab, and an ILT2-neutralizing agent (e.g. an ILT2-neutralizing antibody), for use in the treatment of HNSCC. Such a combination treatment can be useful to relieve the inhibition of NK and CD8 T cell cytotoxicity, and/or to potentiate and/or enhance NK and CD8 T cell cytotoxicity towards tumor cells. In one embodiment, the combination treatment of the disclosure can be particularly advantageous when further combined with administration of an agent that enhances the activity of NK and/or CD8 T cells, for example an antibody that neutralizes PD-1 such as an antibody that binds PD-1 or an antibody that binds PD-L1.

In one aspect, the present invention provides methods of treating and/or preventing a HNSCC, methods for potentiating (or enhancing) NK and CD8 T cell cytotoxicity towards tumor cells in an individual, and/or methods for eliciting an anti-tumor immune response in an individual in need thereof, the method comprising treating the individual with an agent (e.g. an antibody) that binds EGFR (e.g. cetuximab) in combination with an agent (e.g. an antibody) that neutralizes the inhibitory activity of ILT-2. In any embodiment, the individual has an HNSCC.

In one embodiment, provided is an agent that binds EGFR (e.g. cetuximab), for use as a medicament, wherein the agent that binds EGFR is administered in combination with an agent (e.g. an antibody) that neutralizes the inhibitory activity of ILT-2. In one embodiment, the medicament is for eliciting an anti-tumor immune response in an individual having HNSCC. In one embodiment, the medicament is for potentiating (or enhancing) NK and CD8 T cell cytotoxicity towards tumor cells. In one embodiment, the medicament is for increasing the activity and/or numbers of tumor-infiltrating CD8+ T cells and/or NK cells in an individual.

In one embodiment, provided is an agent that neutralizes the inhibitory activity of ILT2 (e.g. an antibody), for use in the treatment of cancer, wherein the agent that neutralizes ILT-2 is used in combination with an antibody that binds EGFR (e.g., an antibody that inhibits EGFR signaling, an antibody that inhibits binding of EGF to EGFR, cetuximab).

In any aspect, the agent that neutralizes the inhibitory activity of ILT-2 and the antibody that binds EGFR are used to treat an individual in further combination with an agent that neutralizes the inhibitory activity of PD-1, e.g., an anti-PD-1 or anti-PDL1 antibody that inhibits the interaction between PD-1 and PDL1.

In any aspect, the antibody that binds EGFR comprises an Fc domain or portion thereof that binds to a human CD16A polypeptide, wherein such antibody is capable of mediating ADCC toward a cell (e.g. an HNSCC cell) that expresses EGFR. In any aspect, the antibody that binds EGFR inhibits EGFR (e.g. inhibits EGFR signaling in a cell). In any aspect, the antibody that binds EGFR inhibits the binding of EGFR to EGF.

In one aspect, the present invention provides methods for treating and/or preventing an HNSCC, methods for potentiating (or enhancing) NK and CD8 T cell cytotoxicity towards tumor cells, and/or methods for eliciting an anti-tumor immune response in an individual in need thereof, wherein said individual has a tumor or cancer characterized by tumor cells that lack or have low expression (e.g. cell surface expression) of HLA-A2 and/or HLA-G polypeptides, the method comprising treating an individual having a cancer with an antibody that binds EGFR in combination with an agent (e.g. an antibody) that neutralizes the inhibitory activity of ILT-2.

The ability to treat HNSCC independently of HLA-A2 and/or HLA-G polypeptide expression permits HNSCC to be treated without restricting treatment to individuals who have HLA-A2 and/or HLA-G positive cancers. In one aspect, the present invention provides a method of treating an individual having an HNSCC without (or without the requirement of) a prior step of assessing the expression of HLA-A2 and/or HLA-G polypeptides, the method comprising treating said individual with an antibody that binds EGFR in combination with an agent (e.g. an antibody) that neutralizes the inhibitory activity of ILT-2. In one aspect, the present invention provides a method of treating an individual having an HNSCC without (or without the requirement of) a prior step of assessing the expression level of HLA-A2 and/or HLA-G polypeptides, the method comprising treating said individual with an antibody that binds EGFR in combination with an agent (e.g. an antibody) that neutralizes the inhibitory activity of ILT-2.

In one aspect, the present invention provides a method of treating an individual without a prior step of determining whether the individual is suitable for treatment based on tumor cell expression HLA-A2 and/or HLA-G polypeptides, the method comprising treating said individual with an antibody that binds EGFR in combination with an agent (e.g. an antibody) that neutralizes the inhibitory activity of ILT-2.

In one aspect, the present invention provides methods for treating and/or preventing a cancer (e.g. an HNSCC), methods for potentiating (or enhancing) NK and CD8 T cell cytotoxicity towards tumor cells, and/or methods for eliciting an anti-tumor immune response in an individual in need thereof, the method comprising: (i) identifying an individual who has a cancer (e.g. an HNSCC) characterized by low or no detectable expression of HLA-A2 and/or HLA-G polypeptides on tumor cells (e.g. tumor cell membrane), and (ii) administering to the individual an antibody that binds EGFR, an agent (e.g. an antibody or antibody fragment) that neutralizes the inhibitory activity of ILT-2.

In one embodiment, provided is a method of increasing the cytotoxic activity and/or numbers of tumor-infiltrating CD8+ T cells and/or NK cells in an individual, the method comprising administering to the individual an effective amount of an antibody that binds EGFR (e.g. cetuximab), and an effective amount of an agent that neutralizes the inhibitory activity of ILT-2.

Among the agents (e.g., antibodies) that neutralize the inhibitory activity of ILT-2 are included, inter alia, molecules (e.g. an antibody or antibody fragment) that bind ILT-2. The agent that neutralizes ILT2 can be characterized by its ability to potentiate the activity of cytotoxic NK lymphocytes and/or CD8 T cells. The agents that neutralize ILT2 can in another aspect optionally be characterized by its ability to promote the development of an adaptive anti-tumor immune response, notably via the differentiation and/or proliferation of CD8 T cells into cytotoxic CD8 T cells.

In one embodiment, an anti-ILT2 antibody, e.g., an antibody or antibody fragment, comprises an immunoglobulin antigen binding domain, optionally hypervariable region, that specifically binds to a human ILT2 protein. The antibody neutralizes the inhibitory signaling of the ILT2 protein. In any embodiment, the antigen binding domain (or antibody or other protein that comprises such) can be specified as not binding to a human ILT1 protein. In any embodiment, the antigen binding domain (or antibody or other protein that comprises such) can be specified as not binding to a human ILT4 protein. In any embodiment, the antigen binding domain (or antibody or other protein that comprises such) can be specified as not binding to a human ILT5 protein. In any embodiment, the antigen binding domain (or antibody or other protein that comprises such) can be specified as not binding to a human ILT6 protein. In one embodiment, the antibodies do not bind a soluble human ILT6 protein. In any embodiment, the antigen binding domain (or antibody or other protein that comprises such) can be specified as not inhibiting the binding of a soluble human ILT6 protein to HLA class I molecules. In any embodiment, the antigen binding domain (or antibody or other protein that comprises such) can be specified as not binding to any one or more of (e.g., lacking binding to each of) ILT-1, ILT-3, ILT-5, ILT-6, ILT-7, ILT-8, ILT-9, ILT-10 and/or IL-T11 proteins; in one embodiment, the antigen binding domain (or antibody or other protein that comprises such) does not bind to any of the human ILT-1, -4, -5 or -6 proteins (e.g., the wild type proteins, the proteins having the amino acid sequences of SEQ ID NOS: 3, 5, 6 and 7 respectively). In any embodiment herein, any ILT protein (e.g., ILT-2) can be specified to be a protein expressed at the surface of a cell (e.g., a primary or donor cell, an NK cell, a T cell, a DC, a macrophage, a monocyte, a recombinant host cell made to express the protein). In another embodiment herein, any ILT protein (e.g., ILT-2) can be specified to be an isolated, recombinant and/or membrane-bound protein.

Optionally, an anti-ILT2 antibody can be specified as being an antibody fragment, a full-length antibody, a multi-specific or bi-specific antibody, that specifically binds to a human ILT2 polypeptide and neutralizes the inhibitory activity of the ILT2 polypeptide. Optionally, the ILT2 polypeptide is expressed at the surface of a cell, optionally an effector lymphocyte, an NK cell, a T cell, e.g., a primary NK cell, an NK cell or population of NK cells derived obtained, purified or isolated from a human individual (e.g. without further modification of the cells).

In one aspect, antibodies that specifically bind human ILT2 enhance the activity (e.g., cytotoxicity) of NK cells (e.g., primary NK cells) towards a target cell bearing at its surface a ligand (e.g., a natural ligand; an HLA class I protein) of ILT2, optionally an HLA-A protein, an HLA-B protein, an HLA-F protein, an HLA-G protein. Optionally the target cell additionally bears HLA-E protein at its surface.

In one embodiment, an antibody that neutralizes the inhibitory activity of ILT-2 is an antibody (e.g., an antibody fragment or a protein that comprises such a fragment) that specifically binds human ILT2 and that enhances and/or restores the cytotoxicity of NK cells (primary NK cells) in a standard 4-hour in vitro cytotoxicity assay in which NK cells that express ILT2 are incubated with target cells that express a ligand (e.g., a natural ligand; an HLA protein, HLA-G protein) of ILT2. In one embodiment the target cells are labeled with ⁵¹Cr prior to addition of NK cells, and then the killing (cytotoxicity) is estimated as proportional to the release of ⁵¹Cr from the cells to the medium. In one embodiment, an antibody that neutralizes the inhibitory activity of ILT-2 is an antibody (e.g., an antibody fragment or a protein that comprises such a fragment) that specifically binds human ILT2 and that enhances expression of cytotoxicity markers CD107 or CD137 at the surface of NK cells when NK cells that express ILT2 are incubated with target cells that express a ligand of ILT2.

In one embodiment, the antibody or antibody fragment is capable of restoring cytotoxicity of NK cells that express ILT2 to at least the level observed with NK cells that do not express ILT2 (e.g., as determined according to the methods of the Examples herein). In one embodiment, the target cells are K562 cells made to express HLA-G, optionally further K562 cells made to express both HLA-G and HLA-E. In one embodiment, the target cells are HNSCC cells, optionally HN, Cal27 cells or FaDu cells.

In any aspect herein, NK cells (e.g., primary NK cells) can be specified as being fresh NK cells purified from human donors, optionally incubated overnight at 37° C. before use. In any aspect herein, NK cells or primary NK cells can be specified as being ILT2 expressing, e.g., for use in assays the cells can be gated on ILT2 by flow cytometry.

In another aspect of any embodiment herein, the antibodies that bind ILT2 can be characterized as being capable of inhibiting (decreasing) the interactions between ILT2 and a HLA class I ligand(s) thereof, particularly a HLA-A, HLA-B, HLA-F and/or HLA-G protein. In one embodiment, the antibodies that bind ILT2 can be characterized as being capable of inhibiting (decreasing) the interactions between ILT2 and a target cell (e.g., tumor cell) that expresses an HLA ligand(s) of ILT-2, particularly a HLA-A, HLA-B, and/or HLA-G protein.

These aspects are more fully described in, and additional aspects, features, and advantages will be apparent from, the description of the invention provided herein.

### BRIEF DESCRIPTION OF THE DRAWINGS

FIG. 1 shows the percent of ILT2 expressing cells in healthy individuals. B lymphocytes and monocytes always express ILT2, conventional CD4 T cells and CD4 Treg cells do not express ILT2, but a significant fraction of CD8 T cells (about 25%), CD3+ CD56+ lymphocytes (about 50%) and NK cells (about 30%) expressed ILT2.

FIGS. 2A to 2F shows the percent of ILT2 expressing cells in cancer patients compared to healthy individuals, showing monocytes (FIG. 2A), B cells (FIG. 2B), CD8 T cells (FIG. 2C), CD4 γδ T cells (FIG. 2D), CD16⁺ NK cells (FIG. 2E) and CD16⁻ NK cells (FIG. 2F). As can be seen, ILT2 was once again expressed on all monocytes and B cells. However on NK cells and CD8 T cell subsets, ILT2 was expressed more frequently with statistical significance on cells from three types of cancers, HNSCC, NSCLC and RCC, compared to the healthy individuals.

FIG. 3 shows % increase in lysis of K562-HLA-G/HLA-E tumor target cells by ILT2-expressing NK cell lines, in presence of antibodies, compared to isotype controls. Antibodies 12D12, 19F10a and commercial 292319 were significantly more effective than other antibodies in the ability to enhance NK cell cytotoxicity.

FIG. 4 shows ability of three exemplary anti-ILT2 antibodies to block the interactions between HLA-G or HLA-A2 expressed at the surface of cell lines and recombinant ILT2 protein was assessed by flow cytometry. 12D12, 18E1 and 26D8 each blocked the interaction of ILT2 with each of HLA-G or HLA-A2.

FIG. 5A is a representative figure showing the increase of % of total NK cells expressing CD137 mediated by anti-ILT2 antibodies using primary NK cells (from two human donors) and K562 tumor target cells made to express HLA-E and HLA-G. FIG. 5B is a representative figure showing the increase of % of ILT2-positive (left hand panel) and ILT2-negative (right hand panel) NK cells expressing CD137 mediated anti-ILT2 antibodies using NK cells from two human donors and HLA-A2-expressing B cell line. In each assay with ILT2-positive NK cells, 12D12, 18E1 and 26D8 potentiated NK cell cytotoxicity to a greater extent that antibody 292319. Each of FIGS. 5A and 5B shows the first donor on the top two panels and the second donor on the bottom two panels.

FIGS. 6A and 6B shows the ability of antibodies to enhance cytotoxicity of primary NK cells toward tumor target cells in terms of fold-increase of cytotoxicity marker CD137. FIG. 6A shows the ability of antibodies to enhance NK cell activation in presence of HLA-G-expressing target cells using primary NK cells from 5-12 different donors against HLA-G and HLA-E expressing K562 target cells. FIG. 6A shows the ability of antibodies to enhance NK cell activation in presence of HLA-G-expressing target cells using primary NK cells from 3-14 different donors against HLA-A2 expressing target B cells. In each case 12D12, 18E1 and 26D8 had greater enhancement of NK cytotoxicity.

FIG. 7 shows a representative example binding of the antibodies to a subset of the ILT2 domain fragment proteins anchored to the cell surface, as assessed by flow cytometry.

FIG. 8A shows a representative example of titration of antibodies 3H5, 12D12 and 27H5 for binding to mutant ILT2 proteins (mutants 1 and 2) anchored to cells, by flow cytometry, showing the these antibodies lost binding to mutants 2. FIG. 8B shows titration of antibodies 26D8, 18E1 and 27C10 for binding to D4 domain mutants 4-1, 4-1b, 4-2, 4-4 and 4-5 by flow cytometry. Antibodies 26D8 and 18E1 lost binding to mutants 4-1 and 4-2, and 26D8 furthermore lost binding to mutant 4-5, while antibody 18E1 had a decrease in binding (but not complete loss of binding) to mutant 4-5. In contrast, antibody 27C10 which did not potentiate the cytotoxicity of primary NK cells lost binding to mutant 4-5 but retained binding to 4-1 or 4-2.

FIG. 9A shows a model representing a portion of the ILT2 molecule that includes domain 1 (top portion, shaded in dark gray) and domain 2 (bottom, shaded in light gray). FIG. 9B shows a model representing a portion of the ILT2 molecule that includes domain 3 (top portion, shaded in dark gray) and domain 4 (bottom, shaded in light gray).

FIG. 10A shows ability of three exemplary anti-ILT2 antibodies to block the interactions between HLA-G or HLA-A2 expressed at the surface of cell lines and recombinant ILT2 protein as assessed by flow cytometry. All antibodies blocked the interactions between HLA-G or HLA-A2, while control antibody did not. FIG. 10B shows the ability of anti-ILT2 antibodies to enhance NK-cell mediated ADCC, determined by assessing cytotoxicity of primary NK cells toward tumor target cells in terms of fold-increase of cytotoxicity marker CD137. While antibodies 12D12, 2H2B, 48F12, and 3F5 were effective in increasing NK cell cytotoxicity, 1A9, 1E4C and 3A7A were not.

FIGS. 11A, 11B, 11C and 11D shows the ability of anti-ILT2 antibodies 12D12, 18E1 and 26D8 to enhance NK-cell mediated ADCC, determined by assessing cytotoxicity of primary NK cells toward tumor target cells in terms of fold-increase of cytotoxicity marker CD137. FIG. 11A shows the ability of antibodies 12D12, 18E1 and 26D8 to enhance the NK cell activation of primary NK cells mediated by rituximab against tumor target cells, in 3 different human NK cell donors. FIGS. 11B, 11C and 11D show the ability of antibodies 12D12, 18E1 and 26D8 to enhance the NK cell activation of primary NK cells mediated by cetuximab against HN (FIG. 11B), FaDu (FIG. 110) or Ca127 (FIG. 11D) HNSCC tumor target cells, in each case in 3 different human NK cell donors.

FIG. 12 shows HNSCC tumor cells were found to be consistently negative for HLA-G and HLA-A2, as determined by flow cytometry, but positive for staining with an antibody reactive broadly against HLA-A, B and C alleles.

FIG. 13 shows enhancement of ADCP by macrophages towards HLA-A2-expressing B cells by ILT2-blocking antibodies in either mouse IgG2b format that is capable of binding to human Fcγ receptors, or in HUB3 format that is not capable of binding to human Fcγ receptors. Results are shown in terms of fold-increase, in combination with the anti-CD20 antibody rituximab.

### DETAILED DESCRIPTION

As used in the specification, “a” or “an” may mean one or more.

Where “comprising” is used, this can optionally be replaced by “consisting essentially of” or by “consisting of”.

Human ILT2 is a member of the lymphocyte inhibitory receptor or leukocyte immunoglobulin- (Ig-) like receptor (LIR/LILRs) family. ILT-2 includes 6 isoforms. Uniprot identifier number Q8NHL6, the entire disclosure of which is incorporated herein by reference, is referred to as the canonical sequence, comprises 650 amino acids, and has the following amino acid sequence (including the signal sequence of residues 1-23):

(SEQ ID NO: 1)
MTPILTVLIC LGLSLGPRTH VQAGHLPKPT LWAEPGSVIT QGSPVTLRCQ GGQETQEYRL
YREKKTALWI TRIPQELVKK GQFPIPSITW EHAGRYRCYY GSDTAGRSES SDPLELVVTG
AYIKPTLSAQ PSPVVNSGGN VILQCDSQVA FDGFSLCKEG EDEHPQCLNS QPHARGSSRA
IFSVGPVSPS RRWWYRCYAY DSNSPYEWSL PSDLLELLVL GVSKKPSLSV QPGPIVAPEE
TLTLQCGSDA GYNRFVLYKD GERDFLQLAG AQPQAGLSQA NFTLGPVSRS YGGQYRCYGA
HNLSSEWSAP SDPLDILIAG QFYDRVSLSV QPGPTVASGE NVTLLCQSQG WMQTFLLTKE
GAADDPWRLR STYQSQKYQA EFPMGPVTSA HAGTYRCYGS QSSKPYLLTH PSDPLELVVS
GPSGGPSSPT TGPTSTSGPE DQPLTPTGSD PQSGLGRHLG VVIGILVAVI LLLLLLLLLF
LILRHRRQGK HWTSTQRKAD FQHPAGAVGP EPTDRGLQWR SSPAADAQEE NLYAAVKHTQ
PEDGVEMDTR SPHDEDPQAV TYAEVKHSRP RREMASPPSP LSGEFLDTKD RQAEEDRQMD
TEAAASEAPQ DVTYAQLHSL TLRREATEPP PSQEGPSPAV PSIYATLAIH.

The ILT2 amino acid sequence without the leader sequence is shown below:

GHLPKPTLWA EPGSVITQGS PVTLRCQGGQ ETQEYRLYRE KKTALWITRI PQELVKK
GQFPIPSITW EHAGRYRCYY GSDTAGRSES SDPLELVVTG AYIKPTLSAQ PSPVVNSGGN
VILQCDSQVA FDGFSLCKEG EDEHPQCLNS QPHARGSSRA IFSVGPVSPS RRWWYRCYAY
DSNSPYEWSL PSDLLELLVL GVSKKPSLSV QPGPIVAPEE TLTLQCGSDA GYNRFVLYKD
GERDFLQLAG AQPQAGLSQA NFTLGPVSRS YGGQYRCYGA HNLSSEWSAP SDPLDILIAG
QFYDRVSLSV QPGPTVASGE NVTLLCQSQG WMQTFLLTKE GAADDPWRLR STYQSQKYQA
EFPMGPVTSA HAGTYRCYGS QSSKPYLLTH PSDPLELVVS GPSGGPSSPT TGPTSTSGPE
DQPLTPTGSD PQSGLGRHLG VVIGILVAVI LLLLLLLLLF LILRHRRQGK HWTSTQRKAD
FQHPAGAVGP EPTDRGLQWR SSPAADAQEE NLYAAVKHTQ PEDGVEMDTR SPHDEDPQAV
TYAEVKHSRP RREMASPPSP LSGEFLDTKD RQAEEDRQMD TEAAASEAPQ DVTYAQLHSL
TLRREATEPP PSQEGPSPAV PSIYATLAIH

### (SEQ ID NO: 2).

In the context of the present invention, “neutralize” or “neutralize the inhibitory activity of ILT2 refers to a process in which an ILT2 protein is inhibited in its capacity to negatively affect intracellular processes leading to immune cell responses (e.g., cytotoxic responses). For example, neutralization of ILT-2 can be measured for example in a standard NK- or T-cell based cytotoxicity assay, in which the capacity of a therapeutic compound to stimulate killing of HLA positive cells by ILT positive lymphocytes is measured. In one embodiment, an antibody preparation causes at least a 10% augmentation in the cytotoxicity of an ILT-2-restricted lymphocyte, optionally at least a 40% or 50% augmentation in lymphocyte cytotoxicity, or optionally at least a 70% augmentation in NK cytotoxicity, and referring to the cytotoxicity assays described. In one embodiment, an antibody preparation causes at least a 10% augmentation in cytokine release by a ILT-2-restricted lymphocyte, optionally at least a 40% or 50% augmentation in cytokine release, or optionally at least a 70% augmentation in cytokine release, and referring to the cytotoxicity assays described. In one embodiment, an antibody preparation causes at least a 10% augmentation in cell surface expression of a marker of cytotoxicity (e.g., CD107 and/or CD137) by a ILT-2-restricted lymphocyte, optionally at least a 40% or 50% augmentation, or optionally at least a 70% augmentation in cell surface expression of a marker of cytotoxicity (e.g., CD107 and/or CD137).

The ability of an anti-ILT2 antibody to “block” or “inhibit” the binding of an ILT2 molecule to a natural ligand thereof (e.g., an HLA molecule) means that the antibody, in an assay using soluble or cell-surface associated ILT2 and natural ligand (e.g., HLA molecule, for example HLA-A, HLA-B, HLA-F, HLA-G), can detectably reduce the binding of a ILT2 molecule to the ligand (e.g., an HLA molecule) in a dose-dependent fashion, where the ILT2 molecule detectably binds to the ligand (e.g., HLA molecule) in the absence of the antibody.

Whenever within this whole specification “treatment of cancer” or the like is mentioned with reference to anti-ILT2 binding agent (e.g., antibody), there is meant: (a) method of treatment of cancer, said method comprising the step of administering (for at least one treatment) an anti-ILT2 binding agent, (preferably in a pharmaceutically acceptable carrier material) to an individual, a mammal, especially a human, in need of such treatment, in a dose that allows for the treatment of cancer, (a therapeutically effective amount), preferably in a dose (amount) as specified herein; (b) the use of an anti-ILT2 binding agent for the treatment of cancer, or an anti-ILT2 binding agent, for use in said treatment (especially in a human); (c) the use of an anti-ILT2 binding agent for the manufacture of a pharmaceutical preparation for the treatment of cancer, a method of using an anti-ILT2 binding agent for the manufacture of a pharmaceutical preparation for the treatment of cancer, comprising admixing an anti-ILT2 binding agent with a pharmaceutically acceptable carrier, or a pharmaceutical preparation comprising an effective dose of an anti-ILT2 binding agent that is appropriate for the treatment of cancer; or (d) any combination of a), b), and c), in accordance with the subject matter allowable for patenting in a country where this application is filed.

As used herein, the term “antigen binding domain” refers to a domain comprising a three-dimensional structure capable of immunospecifically binding to an epitope. Thus, in one embodiment, said domain can comprise a hypervariable region, optionally a VH and/or VL domain of an antibody chain, optionally at least a VH domain. In another embodiment, the binding domain may comprise at least one complementarity determining region (CDR) of an antibody chain. In another embodiment, the binding domain may comprise a polypeptide domain from a non-immunoglobulin scaffold.

The terms “antibody” or “immunoglobulin,” as used interchangeably herein, include whole antibodies and any antigen binding fragment or single chains thereof. A typical antibody comprises at least two heavy (H) chains and two light (L) chains interconnected by disulfide bonds. Each heavy chain is comprised of a heavy chain variable region (V_{H}) and a heavy chain constant region. The heavy chain constant region is comprised of three domains, CH1, CH2, and CH3. Each light chain is comprised of a light chain variable region (V_{L}) and a light chain constant region. The light chain constant region is comprised of one domain, CL. An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids that is primarily responsible for antigen recognition. The terms variable light chain (V_{L}) and variable heavy chain (V_{H}) refer to these light and heavy chains respectively. The heavy-chain constant domains that correspond to the different classes of immunoglobulins are termed “alpha,” “delta,” “epsilon,” “gamma” and “mu,” respectively. Several of these are further divided into subclasses or isotypes, such as IgG1, IgG2, IgG3, IgG4, and the like. The subunit structures and three-dimensional configurations of different classes of immunoglobulins are well known. IgG are the exemplary classes of antibodies employed herein because they are the most common antibodies in the physiological situation and because they are most easily made in a laboratory setting. Optionally the antibody is a monoclonal antibody. Particular examples of antibodies are humanized, chimeric, human, or otherwise-human-suitable antibodies. “Antibodies” also includes any fragment or derivative of any of the herein described antibodies.

The term “specifically binds to” means that an antibody can bind preferably in a competitive binding assay to the binding partner, e.g., ILT2, as assessed using either recombinant forms of the proteins, epitopes therein, or native proteins present on the surface of isolated target cells. Competitive binding assays and other methods for determining specific binding are further described below and are well known in the art.

When an antibody is said to “compete with” a particular monoclonal antibody, it means that the antibody competes with the monoclonal antibody in a binding assay using either recombinant ILT2 molecules or surface expressed ILT2 molecules. For example, if a test antibody reduces the binding of a reference antibody to an ILT2 polypeptide or ILT2-expressing cell in a binding assay, the antibody is said to “compete” respectively with the reference antibody.

The term “affinity”, as used herein, means the strength of the binding of an antibody to an epitope. The affinity of an antibody is given by the dissociation constant Kd, defined as [Ab]×[Ag]/[Ab−Ag], where [Ab−Ag] is the molar concentration of the antibody-antigen complex, [Ab] is the molar concentration of the unbound antibody and [Ag] is the molar concentration of the unbound antigen. The affinity constant Kₐ is defined by 1/Kd. Methods for determining the affinity of mAbs can be found in Harlow, et al., Antibodies: A Laboratory Manual, Cold Spring Harbor Laboratory Press, Cold Spring Harbor, N.Y., 1988), Coligan et al., eds., Current Protocols in Immunology, Greene Publishing Assoc. and Wiley Interscience, N.Y., (1992, 1993), and Muller, Meth. Enzymol. 92:589-601 (1983), which references are entirely incorporated herein by reference. One standard method well known in the art for determining the affinity of mAbs is the use of surface plasmon resonance (SPR) screening (such as by analysis with a BIAcore™ SPR analytical device).

Within the context herein a “determinant” designates a site of interaction or binding on a polypeptide.

The term “epitope” refers to an antigenic determinant, and is the area or region on an antigen to which an antibody binds. A protein epitope may comprise amino acid residues directly involved in the binding as well as amino acid residues which are effectively blocked by the specific antigen binding antibody or peptide, i.e., amino acid residues within the “footprint” of the antibody. It is the simplest form or smallest structural area on a complex antigen molecule that can combine with e.g., an antibody or a receptor. Epitopes can be linear or conformational/structural. The term “linear epitope” is defined as an epitope composed of amino acid residues that are contiguous on the linear sequence of amino acids (primary structure). The term “conformational or structural epitope” is defined as an epitope composed of amino acid residues that are not all contiguous and thus represent separated parts of the linear sequence of amino acids that are brought into proximity to one another by folding of the molecule (secondary, tertiary and/or quaternary structures). A conformational epitope is dependent on the 3-dimensional structure. The term ‘conformational’ is therefore often used interchangeably with ‘structural’.

The term “deplete” or “depleting”, with respect to ILT2-expressing cells means a process, method, or compound that results in killing, elimination, lysis or induction of such killing, elimination or lysis, so as to negatively affect the number of such ILT2-expressing cells present in a sample or in a subject. “Non-depleting”, with reference to a process, method, or compound means that the process, method, or compound is not depleting.

The term “agent” is used herein to denote a chemical compound, a mixture of chemical compounds, a biological macromolecule, or an extract made from biological materials. The term “therapeutic agent” refers to an agent that has biological activity.

For the purposes herein, a “humanized” or “human” antibody refers to an antibody in which the constant and variable framework region of one or more human immunoglobulins is fused with the binding region, e.g., the CDR, of an animal immunoglobulin. Such antibodies are designed to maintain the binding specificity of the non-human antibody from which the binding regions are derived, but to avoid an immune reaction against the non-human antibody. Such antibodies can be obtained from transgenic mice or other animals that have been “engineered” to produce specific human antibodies in response to antigenic challenge (see, e.g., Green et al. (1994) Nature Genet 7:13; Lonberg et al. (1994) Nature 368:856; Taylor et al. (1994) Int Immun 6:579, the entire teachings of which are herein incorporated by reference). A fully human antibody also can be constructed by genetic or chromosomal transfection methods, as well as phage display technology, all of which are known in the art (see, e.g., McCafferty et al. (1990) Nature 348:552-553). Human antibodies may also be generated by in vitro activated B cells (see, e.g., U.S. Pat. Nos. 5,567,610 and 5,229,275, which are incorporated in their entirety by reference).

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

The term “hypervariable region” when used herein refers to the amino acid residues of an antibody that are responsible for antigen binding. The hypervariable region generally comprises amino acid residues from a “complementarity-determining region” or “CDR” (e.g., residues 24-34 (L1), 50-56 (L2) and 89-97 (L3) in the light-chain variable domain and 31-35 (H1), 50-65 (H2) and 95-102 (H3) in the heavy-chain variable domain; Kabat et al. 1991) and/or those residues from a “hypervariable loop” (e.g., residues 26-32 (L1), 50-52 (L2) and 91-96 (L3) in the light-chain variable domain and 26-32 (H1), 53-55 (H2) and 96-101 (H3) in the heavy-chain variable domain; Chothia and Lesk, J. Mol. Biol 1987; 196:901-917), or a similar system for determining essential amino acids responsible for antigen binding. Typically, the numbering of amino acid residues in this region is performed by the method described in Kabat et al., supra. Phrases such as “Kabat position”, “variable domain residue numbering as in Kabat” and “according to Kabat” herein refer to this numbering system for heavy chain variable domains or light chain variable domains. Using the Kabat numbering system, the actual linear amino acid sequence of a peptide may contain fewer or additional amino acids corresponding to a shortening of, or insertion into, a FR or CDR of the variable domain. For example, a heavy chain variable domain may include a single amino acid insert (residue 52a according to Kabat) after residue 52 of CDR H2 and inserted residues (e.g., residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

By “framework” or “FR” residues as used herein is meant the region of an antibody variable domain exclusive of those regions defined as CDRs. Each antibody variable domain framework can be further subdivided into the contiguous regions separated by the CDRs (FR1, FR2, FR3 and FR4).

The terms “Fc domain,” “Fc portion,” and “Fc region” refer to a C-terminal fragment of an antibody heavy chain, e.g., from about amino acid (aa) 230 to about aa 450 of human γ (gamma) heavy chain or its counterpart sequence in other types of antibody heavy chains (e.g., α, δ, ε and μ for human antibodies), or a naturally occurring allotype thereof. Unless otherwise specified, the commonly accepted Kabat amino acid numbering for immunoglobulins is used throughout this disclosure (see Kabat et al. (1991) Sequences of Protein of Immunological Interest, 5th ed., United States Public Health Service, National Institute of Health, Bethesda, Md.).

The terms “isolated”, “purified” or “biologically pure” refer to material that is substantially or essentially free from components which normally accompany it as found in its native state. Purity and homogeneity are typically determined using analytical chemistry techniques such as polyacrylamide gel electrophoresis or high performance liquid chromatography. A protein that is the predominant species present in a preparation is substantially purified.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (nonrecombinant) form of the cell or express native genes that are otherwise abnormally expressed, under expressed or not expressed at all.

Within the context herein, the term antibody that “binds” a polypeptide or epitope designates an antibody that binds said determinant with specificity and/or affinity.

The term “identity” or “identical”, when used in a relationship between the sequences of two or more polypeptides, refers to the degree of sequence relatedness between polypeptides, as determined by the number of matches between strings of two or more amino acid residues. “Identity” measures the percent of identical matches between the smaller of two or more sequences with gap alignments (if any) addressed by a particular mathematical model or computer program (i.e., “algorithms”). Identity of related polypeptides can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al., SIAM J. Applied Math. 48, 1073 (1988).

Methods for determining identity are designed to give the largest match between the sequences tested. Methods of determining identity are described in publicly available computer programs. Computer program methods for determining identity between two sequences include the GCG program package, including GAP (Devereux et al., Nucl. Acid. Res. 12, 387 (1984); Genetics Computer Group, University of Wisconsin, Madison, Wis.), BLASTP, BLASTN, and FASTA (Altschul et al., J. Mol. Biol. 215, 403-410 (1990)). The BLASTX program is publicly available from the National Center for Biotechnology Information (NCBI) and other sources (BLAST Manual, Altschul et al. NCB/NLM/NIH Bethesda, Md. 20894; Altschul et al., supra). The well-known Smith Waterman algorithm may also be used to determine identity.

Anti-EGFR Antibodies

The epidermal growth factor receptor (EGFR; ErbB-1; HER1 in humans) is a transmembrane protein that is a receptor for members of the epidermal growth factor family (EGF family) of extracellular protein ligands. A number of ADCC-mediating anti-EGFR antibodies are known. The anti-EGFR antibody used in accordance with the disclosure can be, for example, an antibody as described in WO2006/082515 and WO2008/017963, WO2002/100348, WO2004/056847, WO2005/056606, WO2005/012479, WO2005/10151, U.S. Pat. No. 6,794,494, EP1454917, WO2003/14159, WO2002/092771, WO2003/12072, WO2002/066058, WO2001/88138, WO98/50433, WO98/36074, WO96/40210, WO 96/27010, US2002065398, WO95/20045, EP586002, U.S. Pat. No. 5,459,061 or 4,943,533, the disclosures of which are incorporated herein by reference. The agent that binds and/or inhibits EGFR may thus be an anti-EGFR antibody, e.g., a chimeric antibody, a human antibody or a humanized antibody. An anti-EGFR antibody used in the method of the present disclosure may have any suitable affinity and/or avidity for one or more epitopes contained in EGFR. Preferably, the antibody used binds to human EGFR with an equilibrium dissociation constant (KD) of at most 10⁻⁸ M, preferably at most 10⁻¹⁰ M. In one embodiment, an anti-EGFR antibody comprises an Fc domain that retains Fcγ (e.g. CD16) binding. In one embodiment, an anti-EGFR antibody comprises a Fc domain of human IgG1 or IgG3 isotype.

An anti-EGFR antibody that comprises an Fc domain or portion thereof will exhibit binding to EGFR via the antigen binding domain and to Fcγ receptors (e.g., CD16A) via the Fc domain. In one embodiment, its ADCC activity toward tumor cells will be mediated at least in part by CD16A. In one embodiment, the additional therapeutic agent is an antibody having a native or modified human Fc domain, for example an Fc domain from a human IgG1 or IgG3 antibody. The term “antibody-dependent cell-mediated cytotoxicity” or “ADCC” is a term well understood in the art, and refers to a cell-mediated reaction in which non-specific cytotoxic cells that express Fc receptors (FcRs) recognize bound antibody on a target cell and subsequently cause lysis of the target cell. Non-specific cytotoxic cells that mediate ADCC include natural killer (NK) cells, macrophages, monocytes, DC and eosinophils. The term “ADCC-inducing antibody” refers to an antibody that demonstrates ADCC as measured by assay(s) known to those of skill in the art. Such activity is typically characterized by the binding of the Fc region with various FcRs. Without being limited by any particular mechanism, those of skill in the art will recognize that the ability of an antibody to demonstrate ADCC can be, for example, by virtue of its subclass (such as IgG1 or IgG3), by mutations introduced into the Fc region, or by virtue of modifications to the carbohydrate patterns in the Fc region of the antibody.

The c225 antibody (cetuximab, ERBITUX®) is an example of an anti-EGFR antibody that can be used in accordance with the methods of the disclosure; cetuximab was demonstrated to inhibit EGF-mediated tumor cell growth in vitro and received marked approval in 2003. Cetuximab binds to the EGFR with an affinity that is approximately 5- to 10-fold higher than that of endogenous ligands. Cetuximab blocks binding of endogenous EGFR ligands resulting in inhibition of the function of the receptor. It is a chimeric human/mouse monoclonal antibody that targets the epidermal growth factor receptor (EGFR). Other anti-EGFR antibodies are known that share some or all of all the biological activities of cetuximab such as preventing ligand binding of the EGFR, preventing activation of the EGFR receptor and the blocking of the downstream signalling of the EGFR pathway resulting in disrupted cell growth. Other examples of antibodies for use in the present disclosure include zalutumumab (2F8, described in WO02/100348 and WO04/056847), nimotuzumab (h-R3), panitumumab (ABX-EGF), and matuzumab (EMD72000), antibodies having the CDRs of the rat ICR62 antibody (WO2010/112413), necitumumab (IMC-11F8, Eli Lilly) or a variant antibody of any of these, or an antibody which is able to compete with any of these, such as an antibody recognizing the same epitope as any of these. Competition may be determined by any suitable technique. In one embodiment, competition is determined by an ELISA assay. Often competition is marked by a significantly greater relative inhibition than 5%, 10% or 25%, as determined by ELISA analysis. Cetuximab can be administered at a dose of 250 mg/m² weekly, optionally wherein cetuximab is administered at a dose of 400 mg/m² as an initial dose, followed by at least one dose at 250 mg/m² weekly.

The Cetuximab heavy and light chain amino acid sequences are shown below.

Cetuximab heavy chain:
(SEQ ID NO: 214)
QVQLKQSGPGLVQPSQSLSITCTVSGFSLTNYGVHWVRQSPGKGLEWLGV
IWSGGNTDYNTPFTSRLSINKDNSKSQVFFKMNSLQSNDTAIYYCARALT
YYDYEFAYWGQGTLVTVSAASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSPGK
Cetuximab light chain:
(SEQ ID NO: 215)
DILLTQSPVILSVSPGERVSFSCRASQSIGTNIHWYQQRTNGSPRLLIKY
ASESISGIPSRFSGSGSGTDFTLSINSVESEDIADYYCQQNNNWPTTFGA
GTKLELKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

Anti-ILT2 Antibodies

An anti-ILT-2 antibody that neutralizes the inhibitory activity of ILT-2 binds an extracellular portion of human ILT-2 receptor and reduces the inhibitory activity of human ILT2 receptor expressed on the surface of an ILT2 positive cell, e.g. an NK cell. In one embodiment the agent competes with HLA-G in binding to ILT-2, i.e. the agent blocks the interaction between ILT-2 and an HLA ligand thereof (e.g. HLA-G).

The starting point for anti-ILT2 antibodies that can then be tested for ILT-2 neutralization activity can include for example produced by classical immunization protocols (e.g. in mice or rats) or selected from libraries of immunoglobulins or immunoglobulin sequences, as disclosed for instance in (Ward et al. Nature, 341 (1989) p. 544). Antibodies can be titrated on ILT2 proteins for the concentration required to achieve maximal binding to a ILT2 polypeptide. Once antibodies are identified that are capable of binding ILT2 and/or having other desired properties, they will also typically be assessed, using standard methods including those described herein, for their ability to bind to other polypeptides, including other ILT2 polypeptides and/or unrelated polypeptides. Ideally, the antibodies only bind with substantial affinity to ILT2 and do not bind at a significant level to unrelated polypeptides or to other ILT proteins, notably ILT-1, -3, -4, -5, -6, -7, and/or -8). However, it will be appreciated that, as long as the affinity (e.g., KD as determined by SPR) for ILT2 is substantially greater (e.g., 10×, 100×, 1000×, 10,000×, or more) than it is for other ILTs and/or other, unrelated polypeptides), then the antibodies are suitable for use in the present methods.

In any embodiment herein, an antibody can be characterized by a KD for binding affinity of less than 1×10⁻⁸ M, optionally less than 1×10⁻⁹ M, or of about 1×10⁻⁸ M to about 1×10⁻¹⁰ M, or about 1×10⁻⁹M to about 1×10¹¹ M, for binding to a human a human ILT2 polypeptide. In one embodiment, affinity is monovalent binding affinity. In one embodiment, affinity is bivalent binding affinity.

In any embodiment herein, an antibody can be characterized by a monovalent KD for binding affinity of less than 2 nM, optionally less than 1 nM.

In any embodiment herein, an antibody can be characterized by a 1:1 Binding fit, as determined by SPR. In any embodiment herein, an antibody can be characterized by dissociation or off rate (kd (1/s)) of less than about 1E-2, optionally less than about 1E-3.

In any embodiment herein, binding affinity can be specified to be monovalent binding as determined by surface plasmon resonance (SPR) screening (such as by analysis with a BIAcore™ SPR analytical device). In any embodiment herein, binding affinity can be specified as being determined by SPR, when anti anti-ILT2 antibodies at 1 μg/mL are captured onto a Protein-A chip and recombinant human ILT2 proteins (e.g., tetrameric ILT2 protein) are injected over captured antibodies.

The affinity can be specified as being determined by SPR, when anti anti-ILT2 antibodies at 1 μg/mL are captured onto a Protein-A chip and recombinant human ILT2 proteins were injected over captured antibodies.

The anti-ILT2 antibodies can be prepared as non-depleting antibodies such that they have reduced, or substantially lack, specific binding to human FCγ receptors. Such antibodies may comprise constant regions of various heavy chains that are known not to bind, or to have low binding affinity for CD16 and optionally further other FCγ receptors. One such example is a wild-type human IgG4 constant region which naturally has lowered CD16 binding but retains significant binding to other receptors such as CD64. Alternatively, antibody fragments that do not comprise constant regions, such as Fab or F(ab′)2 fragments, can be used to avoid Fc receptor binding. Fc receptor binding can be assessed according to methods known in the art, including for example testing binding of an antibody to Fc receptor protein in a BIACORE assay. Also, any antibody isotype (e.g. human IgG1, IgG2, IgG3 or IgG4) can be used in which the Fc portion is modified to decrease, minimize or eliminate binding to Fc receptors (see, e.g., WO03101485). Assays such as, e.g., cell based assays, to assess Fc receptor binding are well known in the art, and are described in, e.g., WO03101485.

Cross-blocking assays can also be used to evaluate whether a test antibody affects the binding of the HLA class I ligand for human ILT2. For example, to determine whether an anti-ILT2 antibody preparation reduces or blocks ILT2 interactions with an HLA class I molecule, the following test can be performed: A dose-range of anti-human ILT2 Fab is co-incubated 30 minutes at room temperature with the human ILT2-Fc at a fixed dose, then added on HLA class 1-ligand expressing cell lines for 1 h. After washing cells two times in staining buffer, a PE-coupled goat anti-mouse IgG Fc fragment secondary antibodies diluted in staining buffer is added to the cells and plates are incubated for 30 additional minutes at 4° C. Cells are washed two times and analyzed on an Accury C6 flow cytometer equipped with an HTFC plate reader. In the absence of test antibodies, the ILT2-Fc binds to the cells. In the presence of an antibody preparation pre-incubated with ILT2-Fc that blocks ILT2-binding to HLA class I, there is a reduced binding of ILT2-Fc to the cells.

In one aspect, the antibodies lack binding to an ILT2 protein modified to lack the D1 domain. In one aspect, the antibodies bind full-length wild-type ILT2 polypeptide but lack binding to an ILT2 protein modified to lack the segment of residues 24 to 121 of the amino acid sequence of SEQ ID NO: 1. In another aspect, the antibodies bind full-length wild-type ILT2 polypeptide but have reduced binding to an ILT2 protein modified to lack the D4 domain. In one aspect, the antibodies bind full-length wild-type ILT2 polypeptide but lack binding to an ILT2 protein modified to lack the segment of residues 322 to 458 of the amino acid sequence of SEQ ID NO: 1.

Binding of anti-ILT2 antibody to cells transfected to express a ILT2 mutant can be measured and compared to the ability of anti-ILT2 antibody to bind cells expressing wild-type ILT2 polypeptide (e.g., SEQ ID NO: 1). A reduction in binding between an anti-ILT2 antibody and a mutant ILT2 polypeptide means that there is a reduction in binding affinity (e.g., as measured by known methods such FACS testing of cells expressing a particular mutant, or by Biacore™ (SPR) testing of binding to mutant polypeptides) and/or a reduction in the total binding capacity of the anti-ILT antibody (e.g., as evidenced by a decrease in B max in a plot of anti-ILT2 antibody concentration versus polypeptide concentration). A significant reduction in binding indicates that the mutated residue is directly involved in binding to the anti-ILT2 antibody or is in close proximity to the binding protein when the anti-ILT2 antibody is bound to ILT2.

In some embodiments, a significant reduction in binding means that the binding affinity and/or capacity between an anti-ILT2 antibody and a mutant ILT2 polypeptide is reduced by greater than 40%, greater than 50%, greater than 55%, greater than 60%, greater than 65%, greater than 70%, greater than 75%, greater than 80%, greater than 85%, greater than 90% or greater than 95% relative to binding between the antibody and a wild type ILT2 polypeptide. In certain embodiments, binding is reduced below detectable limits. In some embodiments, a significant reduction in binding is evidenced when binding of an anti-ILT2 antibody to a mutant ILT2 polypeptide is less than 50% (e.g., less than 45%, 40%, 35%, 30%, 25%, 20%, 15% or 10%) of the binding observed between the anti-ILT2 antibody and a wild-type ILT2 polypeptide.

Once an antigen-binding compound having the desired binding for ILT2 is obtained it may be assessed for its ability to inhibit ILT2. For example, if an anti-ILT2 antibody reduces or blocks ILT2 activation induced by a HLA ligand (e.g., as present on a cell), it can increase the cytotoxicity of ILT2-restricted lymphocytes. This can be evaluated by a typical cytotoxicity assay, examples of which are described below.

The ability of an antibody to reduce ILT2-mediated signaling can be tested in a standard 4-hour in vitro cytotoxicity assay using, e.g., NK cells that express ILT2, and target cells that express an HLA ligand of the ILT2. Such NK cells do not efficiently kill targets that express the ligand because ILT2 recognizes the HLA ligand, leading to initiation and propagation of inhibitory signaling that prevents lymphocyte-mediated cytolysis. Such an assay can be carried out using primary NK cells, e.g., fresh NK cells purified from donors, incubated overnight at 37° C. before use. Such an in vitro cytotoxicity assay can be carried out by standard methods that are well known in the art, as described for example in Coligan et al., eds., Current Protocols in Immunology, Greene Publishing Assoc. and Wiley Interscience, N.Y., (1992, 1993). The target cells are labeled with ⁵¹Cr prior to addition of NK cells, and then the killing is estimated as proportional to the release of ⁵¹Cr from the cells to the medium, as a result of killing. The addition of an antibody that prevents ILT2 protein from binding to the HLA class I ligand (e.g. HLA-G) results in prevention of the initiation and propagation of inhibitory signaling via the ILT2 protein. Therefore, addition of such agents results in increases in lymphocyte-mediated killing of the target cells. This step thereby identifies agents that prevent ILT2-mediated negative signaling by, e.g., blocking ligand binding. In a particular ⁵¹Cr-release cytotoxicity assay, ILT2-expressing NK effector-cells can kill HLA ligand-negative target cells, but less well HLA ligand-expressing control cells. Thus, NK effector cells kill less efficiently HLA ligand positive cells due to HLA-induced inhibitory signaling via ILT2. When NK cells are pre-incubated with blocking anti-ILT2 antibodies in such a ⁵¹Cr-release cytotoxicity assay, HLA ligand-expressing cells are more efficiently killed, in an antibody-concentration-dependent fashion.

The inhibitory activity (i.e., cytotoxicity enhancing potential) of an antibody can also be assessed in any of a number of other ways, e.g., by its effect on intracellular free calcium as described, e.g., in Sivori et al., J. Exp. Med. 1997; 186: 1129-1136, the disclosure of which is herein incorporated by reference, or by the effect on markers of NK cell cytotoxicity activation, such as degranulation marker CD107 or CD137 expression. NK or CD8 T cell activity can also be assessed using any cell based cytotoxicity assays, e.g., measuring any other parameter to assess the ability of the antibody to stimulate NK cells to kill target cells such as P815, K562 cells, or appropriate tumor cells as disclosed in Sivori et al., J. Exp. Med. 1997; 186:1129-1136; Vitale et al., J. Exp. Med. 1998; 187:2065-2072; Pessino et al. J. Exp. Med. 1998; 188:953-960; Neri et al. Clin. Diag. Lab. Immun. 2001; 8:1131-1135; Pende et al. J. Exp. Med. 1999; 190:1505-1516, the entire disclosures of each of which are herein incorporated by reference.

In one embodiment, an antibody preparation causes at least a 10% augmentation in the cytotoxicity of an ILT2-restricted lymphocyte, preferably at least a 30%, 40% or 50% augmentation in NK cytotoxicity, or more preferably at least a 60% or 70% augmentation in NK cytotoxicity.

The activity of a cytotoxic lymphocyte can also be addressed using a cytokine-release assay, wherein NK cells are incubated with the antibody to stimulate the cytokine production of the NK cells (for example IFN-γ and TNF-α production). In an exemplary protocol, IFN-γ production from PBMC is assessed by cell surface and intracytoplasmic staining and analysis by flow cytometry after 4 days in culture. Briefly, Brefeldin A (Sigma Aldrich) is added at a final concentration of 5 μg/ml for the last 4 hours of culture. The cells are then incubated with anti-CD3 and anti-CD56 mAb prior to permeabilization (IntraPrep™; Beckman Coulter) and staining with PE-anti-IFN-γ or PE-IgG1 (Pharmingen). GM-CSF and IFN-γ production from polyclonal activated NK cells are measured in supernatants using ELISA (GM-CSF: DuoSet Elisa, R&D Systems, Minneapolis, Minn., IFN-γ: OptEIA set, Pharmingen).

In one approach, antibodies can optionally be identified and selected based on binding to the same region or epitope on the surface of the ILT2 polypeptide as any of the antibodies described herein, e.g., 12D12, 26D8, 18E1, 2A8A, 2A9, 2C4, 2C8, 2D8, 2E2B, 2E2C, 2E8, 2E11, 2H2A, 2H2B, 2H12, 1A10D, 1E4B, 3E5, 3E7A, 3E7B, 3E9B, 3F5, 4C11B, 4E3A, 4E3B, 4H3, 5D9, 6C6 or 48F12 (e.g. an epitope- or binding region-directed screen). In one aspect, the antibodies bind substantially the same epitope as any of antibodies 12D12, 26D8, 18E1, 2A8A, 2A9, 2C4, 2C8, 2D8, 2E2B, 2E2C, 2E8, 2E11, 2H2A, 2H2B, 2H12, 1A10D, 1E4B, 3E5, 3E7A, 3E7B, 3E9B, 3F5, 4C11B, 4E3A, 4E3B, 4H3, 5D9, 6C6 or 48F12. In one embodiment, the antibodies bind to an epitope of ILT2 that at least partially overlaps with, or includes at least one residue in, the epitope bound by antibody 12D12, 26D8, 18E1, 2A8A, 2A9, 2C4, 2C8, 2D8, 2E2B, 2E2C, 2E8, 2E11, 2H2A, 2H2B, 2H12, 1A10D, 1E4B, 3E5, 3E7A, 3E7B, 3E9B, 3F5, 4C11B, 4E3A, 4E3B, 4H3, 5D9, 6C6 or 48F12. The residues bound by the antibody can be specified as being present on the surface of the ILT2 polypeptide, e.g., on an ILT2 polypeptide expressed on the surface of a cell.

Binding of anti-ILT2 antibody to a particular site on ILT2 can be assessed by measuring binding of an anti-ILT2 antibody to cells transfected with ILT2 mutants, as compared to the ability of anti-ILT2 antibody to bind wild-type ILT2 polypeptide (e.g., SEQ ID NO: 1). A reduction in binding between an anti-ILT2 antibody and a mutant ILT2 polypeptide (e.g., a mutant of Table 6) means that there is a reduction in binding affinity (e.g., as measured by known methods such FACS testing of cells expressing a particular mutant, or by Biacore testing of binding to mutant polypeptides) and/or a reduction in the total binding capacity of the anti-ILT2 antibody (e.g., as evidenced by a decrease in B max in a plot of anti-ILT2 antibody concentration versus polypeptide concentration). A significant reduction in binding indicates that the mutated residue is directly involved in binding to the anti-ILT2 antibody or is in close proximity to the binding protein when the anti-ILT2 antibody is bound to ILT2.

In some embodiments, a significant reduction in binding means that the binding affinity and/or capacity between an anti-ILT2 antibody and a mutant ILT2 polypeptide is reduced by greater than 40%, greater than 50%, greater than 55%, greater than 60%, greater than 65%, greater than 70%, greater than 75%, greater than 80%, greater than 85%, greater than 90% or greater than 95% relative to binding between the antibody and a wild type ILT2 polypeptide. In certain embodiments, binding is reduced below detectable limits. In some embodiments, a significant reduction in binding is evidenced when binding of an anti-ILT2 antibody to a mutant ILT2 polypeptide is less than 50% (e.g., less than 45%, 40%, 35%, 30%, 25%, 20%, 15% or 10%) of the binding observed between the anti-ILT2 antibody and a wild-type ILT2 polypeptide.

In some embodiments, anti-ILT2 antibodies are provided that exhibit significantly lower binding for a mutant ILT2 polypeptide in which a residue in a segment comprising an amino acid residue bound by antibody 12D12, 26D8, 18E1, 2A8A, 2A9, 2C4, 2C8, 2D8, 2E2B, 2E2C, 2E8, 2E11, 2H2A, 2H2B, 2H12, 1A10D, 1E4B, 3E5, 3E7A, 3E7B, 3E9B, 3F5, 4C11B, 4E3A, 4E3B, 4H3, 5D9, 6C6 or 48F12 is substituted with a different amino acid, compared to a binding to a wild-type ILT2 polypeptide not comprising such substitution(s) (e.g. a polypeptide of SEQ ID NO: 1).

In some embodiments, anti-ILT2 antibodies (e.g., other than 12D12, 26D8 or 18E1) are provided that bind the epitope on ILT2 bound by antibody 12D12, 26D8 or 18E1.

In any embodiment herein, an antibody can be characterized as an antibody other than GHI/75, 292319, HP-F1, 586326 and 292305 (or an antibody sharing the CDRs thereof).

In one aspect, an anti-ILT2 antibody binds an epitope positioned on or within the D1 domain (domain 1) of the human ILT2 protein. In one aspect, an anti-ILT2 antibody competes with antibody 12D12 for binding to an epitope on the D1 domain (domain 1) of the human ILT2 protein.

The D1 domain can be defined as corresponding or having the amino acid sequence as follows:

(SEQ ID NO: 55
GHLPKPTLWAEPGSVITQGSPVTLRCQGGQETQEYRLYREKKTALWITRI
PQELVKKGQFPIPSITWEHAGRYRCYYGSDTAGRSESSDPLELVVTGA.

In one aspect, the anti-ILT2 antibody has reduced binding, optionally loss of binding, to an ILT2 polypeptide having a mutation at a residue selected from the group consisting of: E34, R36, Y76, A82 and R84 (with reference to SEQ ID NO: 2); optionally, the mutant ILT2 polypeptide has the mutations: E34A, R36A, Y76I, A82S, R84L. In one embodiment, an antibody furthermore has reduced binding to a mutant ILT2 polypeptide comprising a mutation at one or more (or all of) residues selected from the group consisting of G29, Q30, Q33, T32 and D80 (with reference to SEQ ID NO: 2), optionally, the mutant ILT2 polypeptide has the mutations: G29S, Q30L, Q33A, T32A, D80H. In one aspect, the anti-ILT2 antibody has reduced binding, optionally loss of binding, to an ILT2 polypeptide having the mutations: G29S, Q30L, Q33A, T32A, E34A, R36A, Y76I, A82S, D80H and R84L. In each case, a decrease or loss of binding can be specified as being relative to binding between the antibody and a wild-type ILT2 polypeptide comprising the amino acid sequence of SEQ ID NO: 2.

In one aspect, the anti-ILT2 antibody binds an epitope on ILT2 comprising an amino acid residue (e.g., one, two, three, four or five of the residues) selected from the group consisting of E34, R36, Y76, A82 and R84 (with reference to SEQ ID NO: 2). In one aspect, the anti-ILT2 antibody binds an epitope on ILT2 comprising an amino acid residue (e.g., one, two, three, four or five of the residues) selected from the group consisting of G29, Q30, Q33, T32 and D80 (with reference to SEQ ID NO: 2). In one aspect, the anti-ILT2 antibody binds an epitope on ILT2 comprising: (i) an amino acid residue (e.g., one, two, three, four or five of the residues) selected from the group consisting of E34, R36, Y76, A82 and R84, and (ii) an amino acid residue (e.g., one, two, three, four or five of the residues) selected from the group consisting of G29, Q30, Q33, T32 and D80. In one aspect, the anti-ILT2 antibody binds an epitope on ILT2 comprising an amino acid residue (e.g., one, two, three, four or five of the residues) selected from the group consisting of G29, Q30, Q33, T32, E34, R36, Y76, A82, D80 and R84.

In one aspect, an anti-ILT2 antibody binds an epitope positioned on or within the D4 domain (domain 4) of the human ILT2 protein. In one aspect, an anti-ILT2 antibody competes with antibody 26D8 and/or 18E1 for binding to an epitope on the D4 domain (domain 4) of the human ILT2 protein.

The D4 domain can be defined as corresponding or having the amino acid sequence as follows:

(SEQ ID NO: 56)
FYDRVSLSVQPGPTVASGENVTLLCQSQGWMQTFLLTKEGAADDPWRLRS
TYQSQKYQAEFPMGPVTSAHAGTYRCYGSQSSKPYLLTHPSDPLELVVSG
PSGGPSSPTTGPTSTSGPEDQPLTPTGSDPQSGLGRH.

In one aspect, the anti-ILT2 antibody has reduced binding, optionally loss of binding, to an ILT2 polypeptide having a mutation at a residue selected from the group consisting of: F299, Y300, D301, W328, Q378 and K381 (with reference to SEQ ID NO: 2); optionally, the mutant ILT2 polypeptide has the mutations: F299I, Y300R, D301A, W328G, Q378A, K381N. In one embodiment, an antibody furthermore has reduced binding to a mutant ILT2 polypeptide comprising a mutation at one or more (or all of) residues selected from the group consisting of W328, Q330, R347, T349, Y350 and Y355 (with reference to SEQ ID NO: 2), optionally, the mutant ILT2 polypeptide has the mutations: W328G, Q330H, R347A, T349A, Y350S, Y355A. In one embodiment, an antibody furthermore has reduced binding to a mutant ILT2 polypeptide comprising a mutation at one or more (or all of) residues selected from the group consisting of D341, D342, W344, R345 and R347 (with reference to SEQ ID NO: 2), optionally, the mutant ILT2 polypeptide has the mutations: D341A, D342S, W344L, R345A, R347A. In one embodiment, an antibody has reduced binding to a mutant ILT2 polypeptide having the mutations: F299I, Y300R, D301A, W328G, Q330H, R347A, T349A, Y350S, Y355A, Q378A and K381N. In one embodiment, an antibody has reduced binding to a mutant ILT2 polypeptide having the mutations F299I, Y300R, D301A, W328G, D341, D342, W344, R345, R347, Q378A and K381N. In one embodiment, an antibody has reduced binding to a mutant ILT2 polypeptide having the mutations: F299I, Y300R, D301A, W328G, Q330H, D341A, D342S, W344L, R345A, R347A, T349A, Y350S, Y355A, Q378A and K381N. In each case, a decrease or loss of binding can be specified as being relative to binding between the antibody and a wild-type ILT2 polypeptide comprising the amino acid sequence of SEQ ID NO: 2.

In one aspect, the anti-ILT2 antibody binds an epitope on ILT2 comprising an amino acid residue (e.g., one, two, three, four or five of the residues) selected from the group consisting of F299, Y300, D301, W328, Q378 and K381 (with reference to SEQ ID NO: 2). In one aspect, the anti-ILT2 antibody binds an epitope on ILT2 comprising an amino acid residue (e.g., one, two, three, four or five of the residues) selected from the group consisting of W328, Q330, R347, T349, Y350 and Y355 (with reference to SEQ ID NO: 2). In one aspect, the anti-ILT2 antibody binds an epitope on ILT2 comprising an amino acid residue (e.g., one, two, three, four or five of the residues) selected from the group consisting of D341, D342, W344, R345 and R347 (with reference to SEQ ID NO: 2).

In one aspect, the anti-ILT2 antibody binds an epitope on ILT2 comprising an amino acid residue (e.g., one, two, three, four or five of the residues) selected from the group consisting of: F299, Y300, D301, W328, Q330, D341, D342, W344, R345, R347, T349, Y350, Y355, Q378 and K381.

In one aspect, the anti-ILT2 antibody binds an epitope on ILT2 comprising: (i) an amino acid residue (e.g., one, two, three, four or five of the residues) selected from the group consisting of F299, Y300, D301, W328, Q378 and K381, and (ii) an amino acid residue (e.g., one, two, three, four or five of the residues) selected from the group consisting of Q330, R347, T349, Y350 and Y355. In one aspect, the anti-ILT2 antibody binds an epitope on ILT2 comprising: (i) an amino acid residue (e.g., one, two, three, four or five of the residues) selected from the group consisting of F299, Y300, D301, W328, Q378 and K381, (ii) an amino acid residue (e.g., one, two, three, four or five of the residues) selected from the group consisting of Q330, R347, T349, Y350 and Y355, and (iii) an amino acid residue (e.g., one, two, three, four or five of the residues) selected from the group consisting of D341, D342, W344, R345 and R347.

### Antibody CDR Sequences

The amino acid sequence of the heavy chain variable region of antibody 26D8 is listed as SEQ ID NO: 12 (see also Table A), the amino acid sequence of the light chain variable region is listed as SEQ ID NO: 13 (see also Table A). In a specific embodiment, provided is an antibody that binds essentially the same epitope or determinant as monoclonal antibodies 26D8; optionally the antibody comprises the hypervariable region of antibody 26D8. In any of the embodiments herein, antibody 26D8 can be characterized by the amino acid sequences and/or nucleic acid sequences encoding it. In one embodiment, the monoclonal antibody comprises the Fab or F(ab′)₂ portion of 26D8. Also provided is an antibody or antibody fragment that comprises the heavy chain variable region of 26D8. According to one embodiment, the antibody or antibody fragment comprises the three CDRs of the heavy chain variable region of 26D8. Also provided is an antibody or antibody fragment that further comprises the variable light chain variable region of 26D8 or one, two or three of the CDRs of the light chain variable region of 26D8. The HCDR1, 2, 3 and LCDR1, 2, 3 sequences can optionally be specified as all (or each, independently) being those of the Kabat numbering system, those of the Chotia numbering system, those of the IMGT numbering, or any other suitable numbering system. Optionally any one or more of said light or heavy chain CDRs may contain one, two, three, four or five or more amino acid modifications (e.g. substitutions, insertions or deletions).

In another aspect, provided is an antibody, wherein the antibody or antibody fragment comprises: a HCDR1 region of 26D8 comprising an amino acid sequence EHTIH (SEQ ID NO: 14), or a sequence of at least 3, 4 or 5 contiguous amino acids thereof, optionally wherein one or more of these amino acids may be substituted by a different amino acid; a HCDR2 region of 26D8 comprising an amino acid sequence WFYPGSGSMKYNEKFKD (SEQ ID NO: 15), or a sequence of at least 4, 5, 6, 7, 8, 9 or 10 contiguous amino acids thereof, optionally wherein one or more of these amino acids may be substituted by a different amino acid; a HCDR3 region of 26D8 comprising an amino acid sequence HTNWDFDY (SEQ ID NO: 16), or a sequence of at least 4, 5, 6, 7, 8, 9 or 10 contiguous amino acids thereof, optionally wherein one or more of these amino acids may be substituted by a different amino acid; a LCDR1 region of 26D8 comprising an amino acid sequence KASQSVDYGGDSYMN (SEQ ID NO: 17), or a sequence of at least 4, 5, 6, 7, 8, 9 or 10 contiguous amino acids thereof, optionally wherein one or more of these amino acids may be substituted by a different amino acid; a LCDR2 region of 26D8 comprising an amino acid sequence AASNLES (SEQ ID NO: 18), or a sequence of at least 4, 5, or 6 contiguous amino acids thereof, optionally wherein one or more of these amino acids may be substituted by a different amino acid; a LCDR3 region of 26D8 comprising an amino acid sequence QQSNEEPWT (SEQ ID NO: 19), or a sequence of at least 4, 5, 6, 7, or 8 contiguous amino acids thereof, optionally wherein one or more of these amino acids may be deleted or substituted by a different amino acid.

The amino acid sequence of the heavy chain variable region of antibody 18E1 is listed as SEQ ID NO: 20 (see also Table A), the amino acid sequence of the light chain variable region is listed as SEQ ID NO: 21 (see also Table A). In a specific embodiment, provided is an antibody that binds essentially the same epitope or determinant as monoclonal antibodies 18E1; optionally the antibody comprises the hypervariable region of antibody 18E1. In any of the embodiments herein, antibody 18E1 can be characterized by the amino acid sequences and/or nucleic acid sequences encoding it. In one embodiment, the monoclonal antibody comprises the Fab or F(ab′)₂ portion of 18E1. Also provided is an antibody or antibody fragment that comprises the heavy chain variable region of 18E1. According to one embodiment, the antibody or antibody fragment comprises the three CDRs of the heavy chain variable region of 18E1. Also provided is an antibody or antibody fragment that further comprises the variable light chain variable region of 18E1 or one, two or three of the CDRs of the light chain variable region of 18E1. The HCDR1, 2, 3 and LCDR1, 2, 3 sequences can optionally be specified as all (or each, independently) being those of the Kabat numbering system, those of the Chotia numbering system, those of the IMGT numbering, or any other suitable numbering system. Optionally any one or more of said light or heavy chain CDRs may contain one, two, three, four or five or more amino acid modifications (e.g. substitutions, insertions or deletions).

In another aspect, provided is an antibody, wherein the antibody or antibody fragment comprises: a HCDR1 region of 18E1 comprising an amino acid sequence AHTIH (SEQ ID NO: 22), or a sequence of at least 3 or 4 contiguous amino acids thereof, optionally wherein one or more of these amino acids may be substituted by a different amino acid; a HCDR2 region of 18E1 comprising an amino acid sequence WLYPGSGSIKYNEKFKD (SEQ ID NO: 23), or a sequence of at least 4, 5, 6, 7, 8, 9 or 10 contiguous amino acids thereof, optionally wherein one or more of these amino acids may be substituted by a different amino acid; a HCDR3 region of 18E1 comprising an amino acid sequence HTNWDFDY (SEQ ID NO: 24), or a sequence of at least 4, 5, 6, 7, 8, 9 or 10 contiguous amino acids thereof, optionally wherein one or more of these amino acids may be substituted by a different amino acid; a LCDR1 region of 18E1 comprising an amino acid sequence KASQSVDYGGASYMN (SEQ ID NO: 25), or a sequence of at least 4, 5, 6, 7, 8, 9 or 10 contiguous amino acids thereof, optionally wherein one or more of these amino acids may be substituted by a different amino acid; a LCDR2 region of 18E1 comprising an amino acid sequence AASNLES (SEQ ID NO: 26), or a sequence of at least 4, 5 or 6 10 contiguous amino acids thereof, optionally wherein one or more of these amino acids may be substituted by a different amino acid; a LCDR3 region of 18E1 comprising an amino acid sequence QQSNEEPWT (SEQ ID NO: 27), or a sequence of at least 4, 5, 6 or 7 contiguous amino acids thereof, optionally wherein one or more of these amino acids may be deleted or substituted by a different amino acid.

The amino acid sequence of the heavy chain variable region of antibody 12D12 is listed as SEQ ID NO: 28 (see also Table A), the amino acid sequence of the light chain variable region is listed as SEQ ID NO: 29 (see also Table A). In a specific embodiment, provided is an antibody that binds essentially the same epitope or determinant as monoclonal antibodies 12D12; optionally the antibody comprises the hypervariable region of antibody 12D12. In any of the embodiments herein, antibody 12D12 can be characterized by the amino acid sequences and/or nucleic acid sequences encoding it. In one embodiment, the monoclonal antibody comprises the Fab or F(ab′)₂ portion of 12D12. Also provided is an antibody or antibody fragment that comprises the heavy chain variable region of 12D12. According to one embodiment, the antibody or antibody fragment comprises the three CDRs of the heavy chain variable region of 12D12. Also provided is an antibody or antibody fragment that further comprises the variable light chain variable region of 12D12 or one, two or three of the CDRs of the light chain variable region of 12D12. The HCDR1, 2, 3 and LCDR1, 2, 3 sequences can optionally be specified as all (or each, independently) being those of the Kabat numbering system, those of the Chotia numbering, those of the IMGT numbering, or any other suitable numbering system. Optionally any one or more of said light or heavy chain CDRs may contain one, two, three, four or five or more amino acid modifications (e.g. substitutions, insertions or deletions).

In another aspect, provided is an antibody or antibody fragment, wherein the antibody or antibody fragment comprises: a HCDR1 region of 12D12 comprising an amino acid sequence SYWVH (SEQ ID NO: 30), or a sequence of at least 3 or 4 contiguous amino acids thereof, optionally wherein one or more of these amino acids may be substituted by a different amino acid; a HCDR2 region of 12D12 comprising an amino acid sequence VIDPSDSYTSYNQNFKG (SEQ ID NO: 31), or a sequence of at least 4, 5, 6, 7, 8, 9 or 10 contiguous amino acids thereof, optionally wherein one or more of these amino acids may be substituted by a different amino acid; a HCDR3 region of 12D12 comprising an amino acid sequence GERYDGDYFAMDY (SEQ ID NO: 32), or a sequence of at least 4, 5, 6, 7, 8, 9 or 10 contiguous amino acids thereof, optionally wherein one or more of these amino acids may be substituted by a different amino acid; a LCDR1 region of 12D12 comprising an amino acid sequence RASENIYSNLA (SEQ ID NO: 33), or a sequence of at least 4, 5, 6, 7, 8, 9 or 10 contiguous amino acids thereof, optionally wherein one or more of these amino acids may be substituted by a different amino acid; a LCDR2 region of 12D12 comprising an amino acid sequence AATNLAD (SEQ ID NO: 34), or a sequence of at least 4, 5 or 6 contiguous amino acids thereof, optionally wherein one or more of these amino acids may be substituted by a different amino acid; a LCDR3 region of 12D12 comprising an amino acid sequence QHFWNTPRT (SEQ ID NO: 35), or a sequence of at least 4, 5, 6 or 7 contiguous amino acids thereof, optionally wherein one or more of these amino acids may be deleted or substituted by a different amino acid.

The respective VH and VL and antibodies 3H5, 27C10 and 27H5 are shown in SEQ ID NOS: 36-37, 38-39 and 40-41, respectively. The HCDR1, 2, 3 and LCDR1, 2, 3 sequences of the antibodies can optionally be specified as all (or each, independently) being those of the Kabat numbering system, those of the Chotia numbering, those of the IMGT numbering, or any other suitable numbering system.

In another aspect of any of the embodiments herein, a heavy chain CDR (e.g., CDR1, 2 and/or 3) may be characterized as being encoded by, or derived from, a murine IGHV1 (e.g., a IGHV1-66 or IGHV1-66*01, or a IGHV1-84 or IGHV1-84*01) gene, or by a rat, non-human primate or human gene corresponding thereto, or at least 80%, 90%, 95%, 98% or 99% identical thereto. In another aspect of any of the embodiments herein, a light chain CDR (e.g., CDR1, 2 and/or 3) may be characterized as being encoded by, or derived from, a murine IGKV3 gene (e.g. IGKV3-4 or IGKV3-4*01, or a IGKV3-5 or IGKV3-5*01 gene), or by a rat, non-human primate or human gene corresponding thereto, or at least 80%, 90%, 95%, 98% or 99% identical thereto.

In another aspect of any of the embodiments herein, a heavy chain CDR (e.g., CDR1, 2 and/or 3) may be characterized as being encoded by, or derived from, a murine IGHV2 (e.g., a IGHV1-3 or IGHV1-3*01 gene, or by a rat, non-human primate or human gene corresponding thereto, or at least 80%, 90%, 95%, 98% or 99% identical thereto. In another aspect of any of the embodiments herein, a light chain CDR (e.g., CDR1, 2 and/or 3) may be characterized as being encoded by, or derived from, a murine IGKV10 gene (e.g. IGKV10-96 or IGK10-96*02), or by a rat, non-human primate or human gene corresponding thereto, or at least 80%, 90%, 95%, 98% or 99% identical thereto.

In another aspect of any of the embodiments herein, a heavy chain CDR (e.g., CDR1, 2 and/or 3) may be characterized as being encoded by a murine IGHV1 or IGHV1-84 gene (e.g., IGHV1-84*01) gene. In another aspect of any of the embodiments herein, a light chain CDR (e.g., CDR1, 2 and/or 3) may be characterized as being encoded by a murine IGKV3 or IGKV3-5 gene (e.g., IGKV3-5*01).

In another aspect of any of the embodiments herein, any of the CDRs 1, 2 and 3 of the heavy and light chains of 12D12, 26D8, 18E1, 2A8A, 2A9, 2C4, 2C8, 2D8, 2E2B, 2E2C, 2E8, 2E11, 2H2A, 2H2B, 2H12, 1A10D, 1E4B, 3E5, 3E7A, 3E7B, 3E9B, 3F5, 4C11B, 4E3A, 4E3B, 4H3, 5D9, 6C6 or 48F12 may be characterized by a sequence of at least 4, 5, 6, 7, 8, 9 or 10 contiguous amino acids thereof, and/or as having an amino acid sequence that shares at least 50%, 60%, 70%, 80%, 85%, 90% or 95% sequence identity with the particular CDR or set of CDRs listed in the corresponding SEQ ID NO.

Optionally, in any embodiment, an 12D12, 26D8, 18E1, 2A8A, 2A9, 2C4, 2C8, 2D8, 2E2B, 2E2C, 2E8, 2E11, 2H2A, 2H2B, 2H12, 1A10D, 1E4B, 3E5, 3E7A, 3E7B, 3E9B, 3F5, 4C11B, 4E3A, 4E3B, 4H3, 5D9, 6C6 or 48F12 antibody can be specified as having a heavy chain comprising part or all of an antigen binding region of the respective antibody (e.g. heavy chain CDR1, 2 and 3), fused to an immunoglobulin heavy chain constant region of the human IgG type, optionally a human IgG1, IgG2, IgG3 or IgG4 isotype, optionally further comprising an amino acid substitution to reduce effector function (binding to human Fcγ receptors). Optionally, in any embodiment, an 12D12, 26D8, 18E1, 2A8A, 2A9, 2C4, 2C8, 2D8, 2E2B, 2E2C, 2E8, 2E11, 2H2A, 2H2B, 2H12, 1A10D, 1E4B, 3E5, 3E7A, 3E7B, 3E9B, 3F5, 4C11B, 4E3A, 4E3B, 4H3, 5D9, 6C6 or 48F12 antibody can be specified as having a light chain comprising part or all of an antigen binding region of the respective antibody (e.g. light chain CDR1, 2 and 3), fused to an immunoglobulin light chain constant region of the human kappa type.

The amino acid sequence of the respective heavy and light chain variable regions of antibodies 2A8A, 2A9, 2C4, 2C8, 2D8, 2E2B, 2E2C, 2E8, 2E11, 2H2A, 2H2B, 2H12, 1A10D, 1E4B, 3E5, 3E7A, 3E7B, 3E9B, 3F5, 4C11B, 4E3A, 4E3B, 4H3, 5D9, 6C6 and 48F12 are listed in Table A. In a specific embodiment, provided is an antibody that binds essentially the same epitope or determinant as monoclonal antibodies 2A8A, 2A9, 2C4, 2C8, 2D8, 2E2B, 2E2C, 2E8, 2E11, 2H2A, 2H2B, 2H12, 1A10D, 1E4B, 3E5, 3E7A, 3E7B, 3E9B, 3F5, 4C11B, 4E3A, 4E3B, 4H3, 5D9, 6C6 or 48F12; optionally the antibody comprises the hypervariable region of antibody 2A8A, 2A9, 2C4, 2C8, 2D8, 2E2B, 2E2C, 2E8, 2E11, 2H2A, 2H2B, 2H12, 1A10D, 1E4B, 3E5, 3E7A, 3E7B, 3E9B, 3F5, 4C11B, 4E3A, 4E3B, 4H3, 5D9, 6C6 or 48F12. In any of the embodiments herein, antibody 26D8 can be characterized by the amino acid sequences and/or nucleic acid sequences encoding it. In one embodiment, the monoclonal antibody comprises the Fab or F(ab′)₂ portion of 2A8A, 2A9, 2C4, 2C8, 2D8, 2E2B, 2E2C, 2E8, 2E11, 2H2A, 2H2B, 2H12, 1A10D, 1E4B, 3E5, 3E7A, 3E7B, 3E9B, 3F5, 4C11B, 4E3A, 4E3B, 4H3, 5D9, 6C6 or 48F12. Also provided is an antibody or antibody fragment that comprises the heavy chain variable region of 2A8A, 2A9, 2C4, 2C8, 2D8, 2E2B, 2E2C, 2E8, 2E11, 2H2A, 2H2B, 2H12, 1A10D, 1E4B, 3E5, 3E7A, 3E7B, 3E9B, 3F5, 4C11B, 4E3A, 4E3B, 4H3, 5D9, 6C6 or 48F12. According to one embodiment, the antibody or antibody fragment comprises the three CDRs of the heavy chain variable region of 2A8A, 2A9, 2C4, 2C8, 2D8, 2E2B, 2E2C, 2E8, 2E11, 2H2A, 2H2B, 2H12, 1A10D, 1E4B, 3E5, 3E7A, 3E7B, 3E9B, 3F5, 4C11B, 4E3A, 4E3B, 4H3, 5D9, 6C6 or 48F12. Also provided is an antibody or antibody fragment that further comprises the variable light chain variable region of 2A8A, 2A9, 2C4, 2C8, 2D8, 2E2B, 2E2C, 2E8, 2E11, 2H2A, 2H2B, 2H12, 1A10D, 1E4B, 3E5, 3E7A, 3E7B, 3E9B, 3F5, 4C11B, 4E3A, 4E3B, 4H3, 5D9, 6C6 or 48F12 or one, two or three of the CDRs of the light chain variable region of 2A8A, 2A9, 2C4, 2C8, 2D8, 2E2B, 2E2C, 2E8, 2E11, 2H2A, 2H2B, 2H12, 1A10D, 1E4B, 3E5, 3E7A, 3E7B, 3E9B, 3F5, 4C11B, 4E3A, 4E3B, 4H3, 5D9, 6C6 or 48F12. The HCDR1, 2, 3 and LCDR1, 2, 3 sequences can optionally be specified as all (or each, independently) being those of the Kabat numbering system, those of the Chotia numbering system, those of the IMGT numbering, or any other suitable numbering system. Optionally any one or more of said light or heavy chain CDRs may contain one, two, three, four or five or more amino acid modifications (e.g. substitutions, insertions or deletions).

In another aspect, provided is an antibody or antibody fragment (or respective VH or VL domain thereof) comprising:

a HCDR1 region (Kabat positions 31-35) of 2H2B comprising an amino acid sequence NYYMQ (SEQ ID NO: 139), or a sequence of at least 3, 4 or 5 contiguous amino acids thereof, optionally wherein one or more of these amino acids may be substituted by a different amino acid, optionally wherein the HCDR1 (or VH) comprises an amino acid substitution at Kabat position 32, 33, 34 and/or 35, optionally wherein the HCDR1 (or VH) comprises at least two aromatic residues (e.g. a Y, H or F) at Kabat position 32, 33, 34 and/or 35, optionally wherein the HCDR1 (or VH) comprises an aromatic residue at Kabat position 32 and/or an aromatic residue, N or Q at 35;

a HCDR2 region (Kabat positions 50-65) of 2H2B comprising an amino acid sequence WIFPGSGESSYNEKFKG (SEQ ID NO: 140) or WIFPGSGESNYNEKFKG (SEQ ID NO: 161), or a sequence of at least 4, 5, 6, 7, 8, 9 or 10 contiguous amino acids thereof, optionally wherein one or more of these amino acids may be substituted by a different amino acid, optionally wherein one or more of these amino acids may be substituted by a different amino acid, optionally wherein the HCDR2 (or VH) comprises an amino acid substitution at Kabat position 52A, 54, 55, 56, 57, 58, 60 and/or 65, optionally wherein the residue at 52A is P or L, optionally wherein the residue at 54 is G, S, N or T, optionally wherein the residue at 55 is G, N or Y, optionally wherein the residue at 56 is E or D, optionally wherein the residue at 57 is S or T, optionally wherein the residue at 58 is S, K or N, optionally wherein the residue at 60 is N or S, optionally wherein the residue at 65 is G or V;

a HCDR3 region (Kabat positions 95-102) of 2H2B comprising an amino acid sequence TWNYDARWGY (SEQ ID NO: 141), or a sequence of at least 4, 5, 6, 7, 8, 9 or 10 contiguous amino acids thereof, optionally wherein one or more of these amino acids may be substituted by a different amino acid, optionally wherein the HCDR3 (or VH) comprises an amino acid substitution at Kabat position 95, optionally wherein the residue at 95 is T or S, optionally wherein the HCDR3 (or VH) comprises an amino acid substitution at Kabat position 101, optionally wherein the residue at 101 is G or V;

a Kabat LCDR1 region (Kabat positions 34-34) of 2H2B comprising an amino acid sequence IPSESIDSYGISFMH (SEQ ID NO: 142), or a sequence of at least 4, 5, 6, 7, 8, 9 or 10 contiguous amino acids thereof, optionally wherein one or more of these amino acids may be substituted by a different amino acid, optionally wherein the LCDR1 (or VL) comprises an amino acid substitution at Kabat position 24, 25, 26, 27, 27A, 28, 33 and/or 34, optionally wherein the residue at 24 is I or R, optionally wherein the residue at 25 is A, P or V, optionally wherein the residue at 26 is S or N, optionally wherein the residue at 27 is E or D, optionally wherein the residue at 27A is S, G, T, I or N, optionally wherein the residue at 28 is Y or F, optionally wherein the residue at 33 is M, I or L, optionally wherein the residue at 34 is H or S, optionally wherein the LCDR1 (or VL) comprises an amino acid deletion at Kabat position 29, 30 31 and/or 32;

a Kabat LCDR2 region (Kabat positions 50-56) of 2H2B comprising an amino acid sequence RASNLES (SEQ ID NO: 143), or a sequence of at least 4, 5, or 6 contiguous amino acids thereof, optionally wherein one or more of these amino acids may be substituted by a different amino acid, optionally wherein one or more of these amino acids may be substituted by a different amino acid, optionally wherein the LCDR2 (or VL) comprises an amino acid substitution at Kabat position 50, 53 and/or 55, optionally wherein the residue at 50 is R or G, optionally wherein the residue at 53 is N, T or I, optionally wherein the residue at 54 is D, E or V;

a Kabat LCDR3 region (Kabat positions 89-97) of 2H2B comprising an amino acid sequence QQSNEDPFT (SEQ ID NO: 144), or a sequence of at least 4, 5, 6, 7, or 8 contiguous amino acids thereof, optionally wherein one or more of these amino acids may be deleted or substituted by a different amino acid, optionally wherein the LCDR3 (or VL) comprises an amino acid substitution at Kabat position 91, 94 and/or 96, optionally wherein the residue at 91 is S or T, optionally wherein the residue at 94 is D or A, optionally wherein the residue at 96 is F or W.

In another aspect, provided is an antibody or antibody fragment comprising: a HCDR1 region of 2A8A comprising an amino acid sequence NFYIH (SEQ ID NO: 145); a HCDR2 region of 2A8A comprising an amino acid sequence WIFPGSGETKFNEKFKV (SEQ ID NO: 146); a HCDR3 region of 2A8A comprising an amino acid sequence SWNYDARWGY (SEQ ID NO: 147); a LCDR1 region of 2A8A comprising an amino acid sequence RASESIDSYGISFLH (SEQ ID NO: 148); a LCDR2 region of 2A8A comprising an amino acid sequence RASNLES (SEQ ID NO: 149); a LCDR3 region of 2A8A comprising an amino acid sequence QQSNEDPFT (SEQ ID NO: 150), Optionally, any CDR sequence can be characterized as a sequence of at least 4, 5, 6 or 7 contiguous amino acids of the listed sequence, optionally wherein one or more of these amino acids may be deleted or substituted by a different amino acid.

In another aspect, provided is an antibody or antibody fragment comprising: a HCDR1 region of 2C4 comprising an amino acid sequence NYYVQ (SEQ ID NO: 151); a HCDR2 region of 2C4 comprising an amino acid sequence WIFPGSGETNYNEKFKA (SEQ ID NO: 152); a HCDR3 region of 2C4 comprising an amino acid sequence TWNYDARWGY (SEQ ID NO: 141); a LCDR1 region of 2C4 comprising an amino acid sequence RPSENIDSYGISFMH (SEQ ID NO: 181); a LCDR2 region of 2C4 comprising an amino acid sequence RASNLES (SEQ ID NO: 149); a LCDR3 region of 2C4 comprising an amino acid sequence QQTNEDPFT (SEQ ID NO: 153), Optionally, any CDR sequence can be characterized as a sequence of at least 4, 5, 6 or 7 contiguous amino acids of the listed sequence, optionally wherein one or more of these amino acids may be deleted or substituted by a different amino acid.

In another aspect, provided is an antibody or antibody fragment comprising: a HCDR1 region of 2E2B comprising an amino acid sequence NYYMQ (SEQ ID NO: 154); a HCDR2 region of 2E2B comprising an amino acid sequence WIFPGGGESNYNEKFKG (SEQ ID NO: 155); a HCDR3 region of 2E2B comprising an amino acid sequence TWNYDARWGY (SEQ ID NO: 141); a LCDR1 region of 2E2B comprising an amino acid sequence IPSESIDSYGISFMH (SEQ ID NO: 156); a LCDR2 region of 2E2B comprising an amino acid sequence RASNLES (SEQ ID NO: 149); a LCDR3 region of 2E2B comprising an amino acid sequence QQSNEDPFT (SEQ ID NO: 150), Optionally, any CDR sequence can be characterized as a sequence of at least 4, 5, 6 or 7 contiguous amino acids of the listed sequence, optionally wherein one or more of these amino acids may be deleted or substituted by a different amino acid.

In another aspect, provided is an antibody or antibody fragment comprising: a HCDR1 region of 2C8 comprising an amino acid sequence NYYIQ (SEQ ID NO: 157); a HCDR2 region of 2C8 comprising an amino acid sequence WIFPGNGETNYNEKFKG (SEQ ID NO: 158); a HCDR3 region of 2C8 comprising an amino acid sequence TWNYDARWGY (SEQ ID NO: 141); a LCDR1 region of 2C8 comprising an amino acid sequence RANESIDSYGISFMH (SEQ ID NO: 159); a LCDR2 region of 2C8 comprising an amino acid sequence RASNLDS (SEQ ID NO: 160); a LCDR3 region of 2C8 comprising an amino acid sequence QQSNEDPFT (SEQ ID NO: 150), Optionally, any CDR sequence can be characterized as a sequence of at least 4, 5, 6 or 7 contiguous amino acids of the listed sequence, optionally wherein one or more of these amino acids may be deleted or substituted by a different amino acid.

In another aspect, provided is an antibody or antibody fragment comprising: a HCDR1 region of 2E2C comprising an amino acid sequence NYYMQ (SEQ ID NO: 154); a HCDR2 region of 2E2C comprising an amino acid sequence WIFPGSGESNYNEKFKG (SEQ ID NO: 161); a HCDR3 region of 2E2C comprising an amino acid sequence TWNYDARWGY (SEQ ID NO: 141); a LCDR1 region of 2E2C comprising an amino acid sequence IPSESIDSYGISFMH (SEQ ID NO: 162); a LCDR2 region of 2E2C comprising an amino acid sequence RASNLES (SEQ ID NO: 149); a LCDR3 region of 2E2C comprising an amino acid sequence QQSNEDPFT (SEQ ID NO: 150), Optionally, any CDR sequence can be characterized as a sequence of at least 4, 5, 6 or 7 contiguous amino acids of the listed sequence, optionally wherein one or more of these amino acids may be deleted or substituted by a different amino acid.

In another aspect, provided is an antibody or antibody fragment comprising: a HCDR1 region of 2A9 comprising an amino acid sequence NYYIH (SEQ ID NO: 163); a HCDR2 region of 2A9 comprising an amino acid sequence WIFPGSGETNYNEKFKV (SEQ ID NO: 164); a HCDR3 region of 2A9 comprising an amino acid sequence TWNYDARWGY (SEQ ID NO: 141); a LCDR1 region of 2A9 comprising an amino acid sequence RASESIDSYGISFMH (SEQ ID NO: 165); a LCDR2 region of 2A9 comprising an amino acid sequence RASNLES (SEQ ID NO: 149); a LCDR3 region of 2A9 comprising an amino acid sequence QQSNEDPFT (SEQ ID NO: 150), Optionally, any CDR sequence can be characterized as a sequence of at least 4, 5, 6 or 7 contiguous amino acids of the listed sequence, optionally wherein one or more of these amino acids may be deleted or substituted by a different amino acid.

In another aspect, provided is an antibody or antibody fragment comprising: a HCDR1 region of 2E11 comprising an amino acid sequence NYYIH (SEQ ID NO: 163); a HCDR2 region of 2E11 comprising an amino acid sequence WIFPGSGDTNYNEKFKG (SEQ ID NO: 166); a HCDR3 region of 2E11 comprising an amino acid sequence TWNYDARWGY (SEQ ID NO: 141); a LCDR1 region of 2E11 comprising an amino acid sequence RVSESIDSYGISFMH (SEQ ID NO: 167); a LCDR2 region of 2E11 comprising an amino acid sequence RASTLES (SEQ ID NO: 168); a LCDR3 region of 2E11 comprising an amino acid sequence QQSNEDPFT (SEQ ID NO: 150), Optionally, any CDR sequence can be characterized as a sequence of at least 4, 5, 6 or 7 contiguous amino acids of the listed sequence, optionally wherein one or more of these amino acids may be deleted or substituted by a different amino acid.

In another aspect, provided is an antibody or antibody fragment comprising: a HCDR1 region of 2E8 comprising an amino acid sequence NFYIH (SEQ ID NO: 145); a HCDR2 region of 2E8 comprising an amino acid sequence WIFPGNGETNYSEKFKG (SEQ ID NO: 169); a HCDR3 region of 2E8 comprising an amino acid sequence TWNYDARWVY (SEQ ID NO: 170); a LCDR1 region of 2E8 comprising an amino acid sequence RASDGIDSYGISFMH (SEQ ID NO: 171); a LCDR2 region of 2E8 comprising an amino acid sequence RASILES (SEQ ID NO: 172); a LCDR3 region of 2E8 comprising an amino acid sequence QQTNEDPFT (SEQ ID NO: 153), Optionally, any CDR sequence can be characterized as a sequence of at least 4, 5, 6 or 7 contiguous amino acids of the listed sequence, optionally wherein one or more of these amino acids may be deleted or substituted by a different amino acid.

In another aspect, provided is an antibody or antibody fragment comprising: a HCDR1 region of 2H12 comprising an amino acid sequence NFYIH (SEQ ID NO: 145); a

HCDR2 region of 2H12 comprising an amino acid sequence WIFPGNGETNYSEKFKG (SEQ ID NO: 173); a HCDR3 region of 2H12 comprising an amino acid sequence TWNYDARWGY (SEQ ID NO: 141); a LCDR1 region of 2H12 comprising an amino acid sequence RASDGIDSYGISFMH (SEQ ID NO: 174); a LCDR2 region of 2H12 comprising an amino acid sequence RASTLES (SEQ ID NO: 168); a LCDR3 region of 2H12 comprising an amino acid sequence QQTNEAPFT (SEQ ID NO: 175), Optionally, any CDR sequence can be characterized as a sequence of at least 4, 5, 6 or 7 contiguous amino acids of the listed sequence, optionally wherein one or more of these amino acids may be deleted or substituted by a different amino acid.

In another aspect, provided is an antibody or antibody fragment comprising: a HCDR1 region of 1E4B comprising an amino acid sequence NYYIN (SEQ ID NO: 176); a HCDR2 region of 1E4B comprising an amino acid sequence WIFPGNGDTNYNEKFKG (SEQ ID NO: 177); a HCDR3 region of 1E4B comprising an amino acid sequence TWNYDARWGY (SEQ ID NO: 141); a LCDR1 region of 1E4B comprising an amino acid sequence RASESIDSYMS (SEQ ID NO: 178); a LCDR2 region of 1E4B comprising an amino acid sequence GASNLES (SEQ ID NO: 179); a LCDR3 region of 1E4B comprising an amino acid sequence QQSNEDPWT (SEQ ID NO: 180), Optionally, any CDR sequence can be characterized as a sequence of at least 4, 5, 6 or 7 contiguous amino acids of the listed sequence, optionally wherein one or more of these amino acids may be deleted or substituted by a different amino acid.

In another aspect, provided is an antibody or antibody fragment comprising: a HCDR1 region of 3E5 comprising an amino acid sequence NFYIH (SEQ ID NO: 145); a HCDR2 region of 3E5 comprising an amino acid sequence WIFPGTGETNFNEKFKV (SEQ ID NO: 182); a HCDR3 region of 3E5 comprising an amino acid sequence SWNYDARWGY (SEQ ID NO: 183); a LCDR1 region of 3E5 comprising an amino acid sequence RASESIDSFGISFMH (SEQ ID NO: 184); a LCDR2 region of 3E5 comprising an amino acid sequence RASNLES (SEQ ID NO: 149); a LCDR3 region of 3E5 comprising an amino acid sequence QQSNEAPFT (SEQ ID NO: 185), Optionally, any CDR sequence can be characterized as a sequence of at least 4, 5, 6 or 7 contiguous amino acids of the listed sequence, optionally wherein one or more of these amino acids may be deleted or substituted by a different amino acid.

In another aspect, provided is an antibody or antibody fragment comprising: a HCDR1 region of 3E7A comprising an amino acid sequence NYYIH (SEQ ID NO: 163); a HCDR2 region of 3E7A comprising an amino acid sequence WIFPGSGETNFNEKFKG (SEQ ID NO: 186); a HCDR3 region of 3E7A comprising an amino acid sequence TWNYDARWGY (SEQ ID NO: 141); a LCDR1 region of 3E7A comprising an amino acid sequence RASESIDSYGISFMH (SEQ ID NO: 187); a LCDR2 region of 3E7A comprising an amino acid sequence RASNLES (SEQ ID NO: 149); a LCDR3 region of 3E7A comprising an amino acid sequence QQSNEDPFT (SEQ ID NO: 150), Optionally, any CDR sequence can be characterized as a sequence of at least 4, 5, 6 or 7 contiguous amino acids of the listed sequence, optionally wherein one or more of these amino acids may be deleted or substituted by a different amino acid.

In another aspect, provided is an antibody or antibody fragment comprising: a HCDR1 region of 3E7A or 3E7B comprising an amino acid sequence NYYIH (SEQ ID NO: 163); a HCDR2 region of 3E7A or 3E7B comprising an amino acid sequence WIFPGSGETNFNEKFKG (SEQ ID NO: 188); a HCDR3 region of 3E7A or 3E7B comprising an amino acid sequence TWNYDARWGY (SEQ ID NO: 141); a LCDR1 region of 3E7A or 3E7B comprising an amino acid sequence RASESIDSYGISFMH (SEQ ID NO: 189); a LCDR2 region of 3E7A or 3E7B comprising an amino acid sequence RASNLES (SEQ ID NO: 149) or RASNLVS (SEQ ID NO: 190); a LCDR3 region of 3E7A or 3E7B comprising an amino acid sequence QQSNEDPFT (SEQ ID NO: 150), Optionally, any CDR sequence can be characterized as a sequence of at least 4, 5, 6 or 7 contiguous amino acids of the listed sequence, optionally wherein one or more of these amino acids may be deleted or substituted by a different amino acid.

In another aspect, provided is an antibody or antibody fragment comprising: a HCDR1 region of 3E9B comprising an amino acid sequence NYYIH (SEQ ID NO: 163); a HCDR2 region of 3E9B comprising an amino acid sequence WIFPGSGETNYNEKFKG (SEQ ID NO: 191); a HCDR3 region of 3E9B comprising an amino acid sequence TWNYDARWGY (SEQ ID NO: 141); a LCDR1 region of 3E9B comprising an amino acid sequence RASETIDSYGISFMH (SEQ ID NO: 192); a LCDR2 region of 3E9B comprising an amino acid sequence RASNLES (SEQ ID NO: 149); a LCDR3 region of 3E9B comprising an amino acid sequence QQSNEDPFT (SEQ ID NO: 150), Optionally, any CDR sequence can be characterized as a sequence of at least 4, 5, 6 or 7 contiguous amino acids of the listed sequence, optionally wherein one or more of these amino acids may be deleted or substituted by a different amino acid.

In another aspect, provided is an antibody or antibody fragment comprising: a HCDR1 region of 3F5 comprising an amino acid sequence NYYIQ (SEQ ID NO: 157); a HCDR2 region of 3F5 comprising an amino acid sequence WIFPGNNETNYNEKFKG (SEQ ID NO: 193); a HCDR3 region of 3F5 comprising an amino acid sequence SWNYDARWGY (SEQ ID NO: 147); a LCDR1 region of 3F5 comprising an amino acid sequence RASEIIDSYGISFMH (SEQ ID NO: 194); a LCDR2 region of 3F5 comprising an amino acid sequence RASNLES (SEQ ID NO: 149); a LCDR3 region of 3F5 comprising an amino acid sequence QQSNEDPFT (SEQ ID NO: 150), Optionally, any CDR sequence can be characterized as a sequence of at least 4, 5, 6 or 7 contiguous amino acids of the listed sequence, optionally wherein one or more of these amino acids may be deleted or substituted by a different amino acid.

In another aspect, provided is an antibody or antibody fragment comprising: a HCDR1 region of 4C11B comprising an amino acid sequence NYYIH (SEQ ID NO: 163); a HCDR2 region of 4C11B comprising an amino acid sequence WIFPGSGETNYSEKFKG (SEQ ID NO: 195); a HCDR3 region of 4C11B comprising an amino acid sequence SWNYDARWGY (SEQ ID NO: 147); a LCDR1 region of 4C11B comprising an amino acid sequence RASESIDSYGISFMH (SEQ ID NO: 196); a LCDR2 region of 4C11B comprising an amino acid sequence RASNLES (SEQ ID NO: 149); a LCDR3 region of 4C11B comprising an amino acid sequence QQSNEDPFT (SEQ ID NO: 150), Optionally, any CDR sequence can be characterized as a sequence of at least 4, 5, 6 or 7 contiguous amino acids of the listed sequence, optionally wherein one or more of these amino acids may be deleted or substituted by a different amino acid.

In another aspect, provided is an antibody or antibody fragment comprising: a HCDR1 region of 4E3A or 4E3B comprising an amino acid sequence NYYIQ (SEQ ID NO: 157); a HCDR2 region of 4E3A or 4E3B comprising an amino acid sequence WIFPGSGETNYNENFKA (SEQ ID NO: 197) or WIFPGSGETNYNENFRA (SEQ ID NO: 198); a HCDR3 region of 4E3A or 4E3B comprising an amino acid sequence TWNYDARWGY (SEQ ID NO: 141); a LCDR1 region of 4E3A or 4E3B comprising an amino acid sequence RPSENIDSYGISFMH (SEQ ID NO: 199); a LCDR2 region of 4E3A or 4E3B comprising an amino acid sequence RASNLES (SEQ ID NO: 149); a LCDR3 region of 4E3A or 4E3B comprising an amino acid sequence QQSNEDPFT (SEQ ID NO: 150), Optionally, any CDR sequence can be characterized as a sequence of at least 4, 5, 6 or 7 contiguous amino acids of the listed sequence, optionally wherein one or more of these amino acids may be deleted or substituted by a different amino acid.

In another aspect, provided is an antibody or antibody fragment comprising: a HCDR1 region of 4H3 comprising an amino acid sequence NYYIH (SEQ ID NO: 163); a HCDR2 region of 4H3 comprising an amino acid sequence WIFPGSGDTNYNEKFKG (SEQ ID NO: 200); a HCDR3 region of 4H3 comprising an amino acid sequence TWNYDARWGY (SEQ ID NO: 141); a LCDR1 region of 4H3 comprising an amino acid sequence RVSESIDSYGISFMH (SEQ ID NO: 201); a LCDR2 region of 4H3 comprising an amino acid sequence RASTLES (SEQ ID NO: 168); a LCDR3 region of 4H3 comprising an amino acid sequence QQSNEDPFT (SEQ ID NO: 150), Optionally, any CDR sequence can be characterized as a sequence of at least 4, 5, 6 or 7 contiguous amino acids of the listed sequence, optionally wherein one or more of these amino acids may be deleted or substituted by a different amino acid.

In another aspect, provided is an antibody or antibody fragment comprising: a HCDR1 region of 5D9 comprising an amino acid sequence NYYIH (SEQ ID NO: 163); a HCDR2 region of 5D9 comprising an amino acid sequence WIFLGSGETNYNEKFKG (SEQ ID NO: 202); a HCDR3 region of 5D9 comprising an amino acid sequence SWNYDARWGY (SEQ ID NO: 147); a LCDR1 region of 5D9 comprising an amino acid sequence RASESIDSYGISFIH (SEQ ID NO: 203); a LCDR2 region of 5D9 comprising an amino acid sequence RASNLES (SEQ ID NO: 149); a LCDR3 region of 5D9 comprising an amino acid sequence QQSNEDPFT (SEQ ID NO: 150), Optionally, any CDR sequence can be characterized as a sequence of at least 4, 5, 6 or 7 contiguous amino acids of the listed sequence, optionally wherein one or more of these amino acids may be deleted or substituted by a different amino acid.

In another aspect, provided is an antibody or antibody fragment comprising: a HCDR1 region of 6C6 comprising an amino acid sequence NFYIH (SEQ ID NO: 145); a HCDR2 region of 6C6 comprising an amino acid sequence WIFPGSGETNYNERFKG (SEQ ID NO: 204); a HCDR3 region of 6C6 comprising an amino acid sequence SWNYDARWGY (SEQ ID NO: 147); a LCDR1 region of 6C6 comprising an amino acid sequence RASESIDSYGISFMH (SEQ ID NO: 205); a LCDR2 region of 6C6 comprising an amino acid sequence RASNLES (SEQ ID NO: 149); a LCDR3 region of 6C6 comprising an amino acid sequence QQSNEDPFT (SEQ ID NO: 150), Optionally, any CDR sequence can be characterized as a sequence of at least 4, 5, 6 or 7 contiguous amino acids of the listed sequence, optionally wherein one or more of these amino acids may be deleted or substituted by a different amino acid.

In another aspect, provided is an antibody or antibody fragment comprising: a HCDR1 region of 2D8 comprising an amino acid sequence NFYIH (SEQ ID NO: 145); a HCDR2 region of 2D8 comprising an amino acid sequence WIFPGSGETNFNEKFKV (SEQ ID NO: 206); a HCDR3 region of 2D8 comprising an amino acid sequence SWNYDARWGY (SEQ ID NO: 147); a LCDR1 region of 2D8 comprising an amino acid sequence RASESVDSYGISFMH (SEQ ID NO: 207); a LCDR2 region of 2D8 comprising an amino acid sequence RASILES (SEQ ID NO: 172); a LCDR3 region of 2D8 comprising an amino acid sequence QQSNEDPFT (SEQ ID NO: 150), Optionally, any CDR sequence can be characterized as a sequence of at least 4, 5, 6 or 7 contiguous amino acids of the listed sequence, optionally wherein one or more of these amino acids may be deleted or substituted by a different amino acid.

In another aspect, provided is an antibody or antibody fragment comprising: a HCDR1 region of 48F12 comprising an amino acid sequence SYGVS (SEQ ID NO: 208); a HCDR2 region of 48F12 comprising an amino acid sequence IIWGDGSTNYHSALVS (SEQ ID NO: 209); a HCDR3 region of 48F12 comprising an amino acid sequence PNWDYYAMDY (SEQ ID NO: 210); a LCDR1 region of 48F12 comprising an amino acid sequence RASQDISNYLN (SEQ ID NO: 211); a LCDR2 region of 48F12 comprising an amino acid sequence YTSRLHS (SEQ ID NO: 212); a LCDR3 region of 48F12 comprising an amino acid sequence QQGITLPLT (SEQ ID NO: 213), Optionally, any CDR sequence can be characterized as a sequence of at least 4, 5, 6 or 7 contiguous amino acids of the listed sequence, optionally wherein one or more of these amino acids may be deleted or substituted by a different amino acid.

In any of the antibodies, e.g., 12D12, 26D8, 18E1, 27C10, 2A8A, 2A9, 2C4, 2C8, 2D8, 2E2B, 2E2C, 2E8, 2E11, 2H2A, 2H2B, 2H12, 1A10D, 1E4B, 3E5, 3E7A, 3E7B, 3E9B, 3F5, 4C11B, 4E3A, 4E3B, 4H3, 5D9, 6C6 or 48F12, the specified variable region and CDR sequences may comprise sequence modifications, e.g. a substitution (1, 2, 3, 4, 5, 6, 7, 8 or more sequence modifications). In one embodiment, any one or more (or all of) CDRs 1, 2 and/or 3 of the heavy and light chains comprises one, two, three or more amino acid substitutions, optionally where the residue substituted is a residue present in a sequence of human origin. In one embodiment the substitution is a conservative modification. A conservative sequence modification refers to an amino acid modification that does not significantly affect or alter the binding characteristics of the antibody containing the amino acid sequence. Such conservative modifications include amino acid substitutions, additions and deletions. Modifications can be introduced into an antibody by standard techniques known in the art, such as site-directed mutagenesis and PCR-mediated mutagenesis. Conservative amino acid substitutions are typically those in which an amino acid residue is replaced with an amino acid residue having a side chain with similar physicochemical properties. Specified variable region and CDR sequences may comprise one, two, three, four or more amino acid insertions, deletions or substitutions. Where substitutions are made, preferred substitutions will be conservative modifications. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g. glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g. threonine, valine, isoleucine) and aromatic side chains (e.g., tyrosine, phenylalanine, tryptophan, histidine). Thus, one or more amino acid residues within the CDR regions of an antibody can be replaced with other amino acid residues from the same side chain family and the altered antibody can be tested for retained function (i.e., the properties set forth herein) using the assays described herein.

Optionally, in any embodiment, a VH may comprise an amino acid substitution at Kabat position 32, 33, 34 and/or 35. A VH may comprise an amino acid substitution at Kabat position 52A, 54, 55, 56, 57, 58, 60 and/or 65. In any embodiment, a VH may comprise an amino acid substitution at Kabat position 95 and/or 101. In any embodiment, a VL may comprise an amino acid substitution at Kabat position 24, 25, 26, 27, 27A, 28, 33 and/or 34, and/or an amino acid deletion at Kabat position 29, 30 31 and/or 32. In any embodiment, a VL may comprise an amino acid substitution at Kabat position 50, 53 and/or 55. In any embodiment, a VL may comprise an amino acid substitution at Kabat position 91, 94 and/or 96.

Optionally, in any embodiment herein, an anti-ILT2 antibody can be characterized as being a function-conservative variant of any of the antibodies, heavy and/or light chains, CDRs or variable regions thereof described herein. “Function-conservative variants” are those in which a given amino acid residue in a protein or antibody has been changed without altering the overall conformation and function of the polypeptide, including, but not limited to, replacement of an amino acid with one having similar properties (such as, for example, polarity, hydrogen bonding potential, acidic, basic, hydrophobic, aromatic, and the like). Amino acids other than those indicated as conserved may differ in a protein so that the percent protein or amino acid sequence similarity between any two proteins of similar function may vary and may be, for example, from 70% to 99% as determined according to an alignment scheme such as by the Cluster Method, wherein similarity is based on the MEGALIGN algorithm. A “function-conservative variant” also includes a polypeptide which has at least 60% amino acid identity as determined by BLAST or FASTA algorithms, preferably at least 75%, more preferably at least 85%, still preferably at least 90%, and even more preferably at least 95%, and which has the same or substantially similar properties or functions as the native or parent protein (e.g. heavy or light chains, or CDRs or variable regions thereof) to which it is compared. In one embodiment, the antibody comprises a heavy chain variable region that is a function-conservative variant of the heavy chain variable region of antibody 2H2B, 48F12, 3F5, 12D12, 26D8 or 18E1, and a light chain variable region that is a function-conservative variant of the light chain variable region of the respective 2H2B, 48F12, 3F5, 12D12, 26D8 or 18E1 antibody. In one embodiment, the antibody comprises a heavy chain that is a function-conservative variant of the heavy chain variable region of antibody 2H2B, 48F12, 3F5, 12D12, 26D8 or 18E1 fused to a human heavy chain constant region disclosed herein, optionally a human IgG4 constant region, optionally a modified IgG (e.g. IgG1) constant region, e.g. a constant region of any of SEQ ID NOS: 42-45, and a light chain that is a function-conservative variant of the light chain variable region of the respective 2H2B, 48F12, 3F5, 12D12, 26D8 or 18E1 antibody fused to a human Ckappa light chain constant region.

TABLE A

| Antibody | SEQ ID | |
|---|---|---|
| domain | NO: | Amino AcidSequence |
| 26D8 VH | 12 | QVQLQQSGAELVKPGASVKLSCKASGYTFTEHTIHWIKQRSGQGLEWIGW |
| | | FYPGSGSMKYNEKFKDKATLTADKSSSTVYMELTRLTSEDSAVYFCARHT |
| | | NWDFDYWGQGTTLTVSS |
| 26D8 VL | 13 | DIVLTQSPASLAVSLGQRATISCKASQSVDYGGDSYMNWYQQKPGQPPKL |
| | | LIYAASNLESGIPARFSGSGSGTDLTLNIHPVEEDDAAMYYCQQSNEEPW |
| | | TFGGGTKLEIK |
| 18E1 VH | 20 | QVQLQQSGAELVKPGASVRLSCKASGYTFTAHTIHWVKQRSGQGLEWIGW |
| | | LYPGSGSIKYNEKFKDKATLTADKSSSTVYMELSRLTSEDSAVYFCARHT |
| | | NWDFDYWGQGTTLTVSS |
| 18E1 VL | 21 | NIVLTQSPASLAVSLGQRATISCKASQSVDYGGASYMNWYQQKPGQPPKL |
| | | LIYAASNLESGIPARFSGSGSGTDLTLNIHPVEEEDAAMYYCQQSNEEPW |
| | | TFGGGTKLEIK |
| 12D12VH | 28 | QVQLQQPGAELVKPGASVRMSCKASGYTFTSYWVHWVKQRPGQGLEWIGV |
| | | IDPSDSYTSYNQNFKGKATLTVDTSSKTAYIHLSSLTSEDSAVYFCARGE |
| | | RYDGDYFAMDYWGQGTSVTVSS |
| 12D12 VL | 29 | DIVMTQSPASLSVSVGETVTITCRASENIYSNLAWYQQKQGKSPQLLVYA |
| | | ATNLADGVPSRFSGSRSGTQYSLKINSLQSEDFGTYYCQHFWNTPRTFGG |
| | | GTKLEIK |
| 3H5 VH | 36 | QVQLKESGPGLVAPSQSLSITCTVSGFSLTSYGVSWVRQPPGKGLEWLGV |
| | | IWGDGSTNYHSALISRLSISKDNSKSQVFLKLNSLQTDDTATYYCAKPRW |
| | | DDYAMDYWGQGTSVTVSS |
| 3H5 VL | 37 | DIQMTQTTSSLSASLGDRVTISCRASQDISNYLNWYQQKPDGTVKLLIYY |
| | | TSRLHSGVPSRFSGSGSGTDYSLTISNLEQEDIATYFCQQGNTLWTFGGG |
| | | TKLEIK |
| 27C10 VH | 38 | EVQLQESGPGLVKPSQSLSLICSVTGYSITSGYYWNWIRQFPENKLEWMG |
| | | YIRYDGSNNYNPSLNNRISITRDASKNQFFLKLNSVTTEDTATYYCARGW |
| | | LLWFYAVDYWGQGTSVTVSS |
| 27C10 VL | 39 | DVVMTQTPLSLPVSLGDQASISCRSSQSIVHTNGNTYLEWYLQKSGQSPK |
| | | LLIYKVSNRLSGVPDRFSGSGSGTDFTLKISRVEAEDLGIYYCFQGSHVP |
| | | WTFGGGTKLEIK |
| 27H5 VH | 40 | QVQLKESGPGLVAPSQSLSITCTVSGFSLTSYGVSWVRQPPGKGLEWLGV |
| | | IWGDGNTNYHSALISRLSISKDNSKSQVFLKLNSLQTDDTATYYCARTNW |
| | | DGWFAYWGQGTLVTVSA |
| 27H5 VL | 41 | DIVMTQSHKFMSTSVGDRVSITCKASQDVGTAVAWYQQKPGQSPKLLIYW |
| | | ASTRHTGVPDRFTGSGSGTDFTLTISNVQSEDLADYFCQQYRSYPLGTFG |
| | | GGTKLEIK |
| 2A8A VH | 81 | QVQLQQSGPELVKPGASVKISCKASGYSFINFYIHWVRQRPGQGLDWIGW |
| | | IFPGSGETKFNEKFKVKATLTADTSSSTAYMQLNSLTSEDSAVYFCARSW |
| | | NYDARWGYWGQGTSVTVSS |
| 2A8A VL | 82 | QIVLTQSPASLAVSLGQRATISCRASESIDSYGISFLHWYQQKPGQPPKL |
| | | LIYRASNLESGIPARFSGSGSRPDFTLTINPVEADDVATYYCQQSNEDPF |
| | | TFGSGTKLEIK |
| 2C4 VH | 83 | DVQLVESGPELVKPGASVKISCKASGYSFTNYYMQWVKQRPGQGLEWIGW |
| | | IFPGGGESNYNEKFKGKATLSADTSSTTAYMQLSSLTSEDSAVYFCARTW |
| | | NYDARWGYWGQGTTVTVSS |
| 2C4 VL | 84 | DIQMTQSPASLTVSLGQRATISCRPSENIDSYGISFMHWYQQKPGQPPKL |
| | | LIYRASNLESGIPVRFSGSGSRTDFTLTINPVEADDVATYYCQQTNEDPF |
| | | TFGSGTKLEIK |
| 2E2B VH | 85 | EVQLKQSGPELVKPGASVKISCKASGYSFTNYYIQWVKQRPGQGLEWIGW |
| | | IFPGNGETNYNEKFKGKATLTADTSSSTAYMQLSSLTSEDSAVYFCARTW |
| | | NYDARWGYWGQGTSVTVSS |
| 2E2B VL | 86 | DIVLTQSPASLAVSLGQRATISCIPSESIDSYGISFMHWYQQKPGQPPKL |
| | | LIYRASNLESGIPARFSGSGSRTDFTLTINPVEADDVATYYCQQSNEDPF |
| | | TFGSGTKLEIK |
| 208 VH | 87 | QVQLQQSGPELVKPGASVKISCKASGYSFTNYYMQWVKQRPGQGLEWIGW |
| | | IFPGSGESNYNEKFKGKATLSADTSSTTAYMQLSSLTSEDSAVYFCARTW |
| | | NYDARWGYWGQGTSVTVSS |
| 208 VL | 88 | DILLTQSPASLTVSLGQRATISCRANESIDSYGISFMHWYQQKPGQPPKL |
| | | LIYRASNLDSGIPARFSGSGSRTDFTLTINPVEADDVATYYCQQSNEDPF |
| | | TFGSGTKLEIK |
| 2E20 VH | 89 | EFQLQQSGPELVKPGASVKISCKASGYSFTNYYMQWVKQRPGQGLEWIGW |
| | | IFPGSGESNYNEKFKGKATLSADTSSTTAYMQLSSLTSEDSAVYFCARTW |
| | | NYDARWGYWGQGTTLTVSS |
| 2E20 VL | 90 | DIVMTQSPASLAVSLGQRATISCIPSESIDSYGISFMHWYQQKPGQPPKL |
| | | LIYRASNLESGIPARFSGSGSRTDFTLTINPVEADDVATYYCQQSNEDPF |
| | | TFGSGTKLEIK |
| 2H2A VH | 91 | EVKLEESGPELVKPGASVKLSCKASGYTFTNYYMQWVKQRPGQGLEWIGW |
| | | IFPGSGESSYNEKFKGKATLSADTSSTTAYMQLSSLTSEDSAVYFCARTW |
| | | NYDARWGYWGQGTTLTVSS |
| 2H2A VL | 92 | DILMTQSPASLAVSLGQRATISCIPSESIDSYGISFMHWYQQKPGQPPKL |
| | | LIYRASNLESGIPARFSGSGSRTDFTLTINPVEADDVATYYCQQSNEDPF |
| | | TFGSGTKLELK |
| 2H2B VH | 93 | EVKLQQSGPELVKPGASVKISCKASGYSFTNYYIHWVKQRPGQGLEWIGW |
| | | IFPGSGETNYNEKFKVKATLSADTSSTTAYMQLSSLTSEDSAVYFCARTW |
| | | NYDARWGYWGQGTTLTVSS |
| 2H2B VL | 94 | DILMTQSPASLAVSLGQRATISCIPSESIDSYGISFMHWYQQKPGQPPKL |
| | | LIYRASNLESGIPARFSGSGSRTDFTLTINPVEADDVATYYCQQSNEDPF |
| | | TFGSGTKLELK |
| 2A9 VH | 95 | QVQLKESGPELVKPGASVKISCKTSGYSFTNYYIHWVKQRPGQGLEWIGW |
| | | IFPGSGDTNYNEKFKGKATLTADTSSNTASMHLSSLTSEDSAVYFCARTW |
| | | NYDARWGYWGQGTTLTVSS |
| 2A9 VL | 96 | DVVVTQTPASLAVSLGQRATISCRASESIDSYGISFMHWYQQKPGQPPKL |
| | | LIYRASNLESGIPARFSGSGSRTDFTLTINPVEADDVATYYCQQSNEDPF |
| | | TFGSGTKLEIK |
| 2E11 VH | 97 | EVQLQQSGPDLVKPGASVKMSCKASGYSFINFYIHWVKQRPGQGLEWIGW |
| | | IFPGNGETNYSEKFKGKATLTADTSSSTAYMQFNSLTYEDSAVYFCARTW |
| | | NYDARWVYWGQGTTVTVSS |
| 2E11 VL | 98 | DIVMTQSPASLAVSLGQRATISCRVSESIDSYGISFMHWYQQKSGQPPKV |
| | | LIYRASTLESGIPARFSGSGSRTDFTLTINPVEADDVATYYCQQSNEDPF |
| | | TFGSGTKLEIK |
| 2E8 VH | 99 | EVKLQQSGPDLVKPGASVKISCKASGYSFINFYIHWVKQRPGQGLEWIGW |
| | | IFPGNGETNYSEKFKGKATLTADTSSSTAYMQFNSLTYEDSAVYFCARTW |
| | | NYDARWGYWGQGTTLTVSS |
| 2E8 VL | 100 | EIVLTQSPASLAVSLGQRATISCRASDGIDSYGISFMHWYQQKPGQPPTV |
| | | LIYRASILESGIPARFSGSGSRTDFTLTINPVEADDVATYYCQQTNEDPF |
| | | TFGSGTKLEIK |
| 2H12 VH | 101 | DVQLVESGPELVKPGASVKISCKASGYSFTNYYMQWVKQRPGQGLEWIGW |
| | | IFPGGGESNYNEKFKGKATLSADTSSTTAYMQLSSLTSEDSAVYFCARTW |
| | | NYDARWGYWGQGTTVTVSS |
| 2H12 VL | 102 | DILLTQSPASLAVSLGQRATISCRASDGIDSYGISFMHWYQQKPGQPPTL |
| | | LIYRASTLESGIPARFSGSGSRTDFTLTINPVEADDVATYYCQQTNEAPF |
| | | TFGSGTKLELK |
| 1E4B VH | 103 | DVQLQESGPELVKPGASVKISCKSSGYSFINFYIHWVKQRPGQGLDWIGW |
| | | IFPGTGETNFNEKFKVKAALTADTSSSTVYMQLSTLTSEDSAVYFCARSW |
| | | NYDARWGYWGQGTSITVSS |
| 1E4B VL | 104 | DVVMTQTPAFLAVSLGQRATISCRASESIDSYMSWYQQKPGQPPKVLIYG |
| | | ASNLESGIPARFSGSGSGTDFTLNIHPVEEEDAATYYCQQSNEDPWTFGG |
| | | GTKLEIK |
| 3E5 VH | 105 | EVQLQESGPELVKPGASVKISCKASGYSFRNYYIQWVKQRPGQGLEWIGW |
| | | IFPGNYETNYNEKFKGKATLSADTSSTTAYMQLSSLTSEDSAVYFCARSW |
| | | NYDARWGYWGQGTSVTVSS |
| 3E5 VL | 106 | ENVLTQSPASLAVSLGQRATISCRASESIDSFGISFMHWYQQKPGQPPKL |
| | | LIYRASNLESGIPARFSGSGSGPDFSLTIDPVEADDVATYYCQQSNEAPF |
| | | TFGSGTKLEIK |
| 1A10D VH | 107 | QVQLKQSGPELVKPGASVKISCKASGYSFTNYYIHWVKQRPGQGLEWIGW |
| | | IFPGSGETNFNEKFKGKATLTADTSSSTAYMQFSSLTSEDSAVYFCARTW |
| | | NYDARWGYWGQGTTVTVSS |
| 1A10D VL | 108 | EIVLTQSPASLAVSLGQRATISCRASESIDSYGISFMHWYQQKPGQPPKL |
| | | LIYRASNLESGIPARFSGSGSRTDFTLTINPVEADDVATYYCQQSNEDPF |
| | | TFGSGTKLEIK |
| 3E7A VH | 109 | QVQLKQSGPELVKPGASVKISCKASGYSFTNYYIHWVKQRPGQGLEWIGW |
| | | IFPGSGETNFNEKFKGKATLTADTSSSTAYMQFSSLTSEDSAVYFCARTW |
| | | NYDARWGYWGQGTTVTVSS |
| 3E7A VL | 110 | DILMTQSPASLAVSLGQRATISCRASEGIDSYGISFMHWYQQKPGQPPTL |
| | | LIYRASNLESGIPARFSGSGSRTDFTLTINPVEADDVATYYCQQTNEDPF |
| | | TFGSGT LEIK |
| 3E7B VH | 111 | EVQLQESGPELVKPGASVKISCKTSGYSFTNYYIHWVKQRPGQGLEWIGW |
| | | IFPGSGETNYNEKFKGKATLSADTSSTTAYMQLSSLTSEDSAVYFCARTW |
| | | NYDARWGYWGQGTTVTVSS |
| 3E7B VL | 112 | EIQMTQSPASLAVSLGQRATISCRASEGIDSYGISFMHWYQQKPGQPPTL |
| | | LIYRASNLVSGIPARFSGSGSRTDFTLTINPVEADDVATYYCQQTNEDPF |
| | | TFGSGTKLEIK |
| 3E9B VH | 113 | DVQLQESGPDLVKPGASVKISCKASGYSFRNYYIQWVKQRPGQGLEWIGW |
| | | IFPGNNETNYNEKFKGKATLSADTSSTTAYMQLSSLTSEDSAVYFCARSW |
| | | NYDARWGYWGQGTTLTVSS |
| 3E9B VL | 114 | EILLTQSPASLAVSLGQRATISCRASETIDSYGISFMHWYQQKPGQPPKL |
| | | LIYRASNLESGIPARFSGSGSRTDFTLTINPVEADDVATYYCQQSNEDPF |
| | | TFGSGTKLEIK |
| 3F5 VH | 115 | QVQLKESGPELVKPGASVKISCKASGYSFTNYYIHWVKQRPGQGLEWIGW |
| | | IFPGSGETNYSEKFKGEAILTADTSSNTAYMQLSSLTSEDSAVYFCARSW |
| | | NYDARWGYWGQGTTLTVSS |
| 3F5 VL | 116 | EIVLTQSPASLAVSLGQRATISCRASEIIDSYGISFMHWYQQKPGQPPKL |
| | | LIYRASNLESGIPARFSGSGSRTDFTLTINPVEADDVATYYCQQSNEDPF |
| | | TFGSGTKLEIK |
| 4C11B VH | 117 | QIQLQQSGPELVKPGASVKISCKASGYSFTNYYIQWVKQRPGQGLEWIGW |
| | | IFPGSGETNYNENFKAKATLSADTSSTTAYMQLSSLTSEDSAVYFCARTW |
| | | NYDARWGYWGQGTSVTVSS |
| 4C11B VL | 118 | QIVLSQSPVSLAVSPGQRATISCRASESIDSYGISFMHWYKQKPGQPPKL |
| | | LIYRASNLESGIPARFSGSGSRTDFTLTINPVEADDVATYYCQQSNEDPF |
| | | TFGSGTKLEIK |
| 4E3A VH | 119 | EVHLQQSGPELVKPGASVKISCKASGYSFTNYYIQWVKQRPGQGLEWIGW |
| | | IFPGSGETNYNENFRAKATLSADTSSTTAYMQLSSLTSEDSAVYFCARTW |
| | | NYDARWGYWGQGTTVTVSS |
| 4E3A VL | 120 | EILLTQSPPASLAVSLGQRVTISCRPSENIDSYGISFMHWYQQKPGQPPK |
| | | LLIYRASNLESGIPVRFSGSGSRTDFILTINPVEADDVATYYCQQSNEDP |
| | | FTFGSGTKLEIK |
| 4E3B VH | 121 | QVQLKESGPELVKPGASVKISCKTSGYIFTNYYIHWVKQRPGQGLEWIGW |
| | | IFPGSGDTNYNEKFKGKATLTADTSSSTASMQLSSLTSEDSAVYFCARTW |
| | | NYDARWGYWGQGTTVTVSS |
| 4E3B VL | 122 | DILLTQSPASLAVSLGQRATISCRPSENIDSYGISFMHWCQQKPGQPPKL |
| | | LIYRASNLESGIPVRFSGSGSRTDFTLTINPVEADDVATYYCQQSNEDPF |
| | | TFGSGTKLEIK |
| 4H3 VH | 123 | QVQLKESGPELVKPGASVKISCKASGYSFTNYYIHWVKQRPGQGLEWIGW |
| | | IFLGSGETNYNEKFKGEAILTADTSSTTAYMQLSSLTSEDSAVYFCARSW |
| | | NYDARWGYWGQGTTLTVSS |
| 4H3 VL | 124 | DILLTQSPASLAVSLGQRATISCRVSESIDSYGISFMHWYQQKSGQPPKV |
| | | LIYRASTLESGIPARFSGSGSRTDFTLTINPVEADDVATYYCQQSNEDPF |
| | | TFGSGTKLEIK |
| 5D9 VH | 125 | EVQLQQSGPELVKPGASVKISCKASGYSFINFYIHWVKQRPGQGLDWIGW |
| | | IFPGSGETNYNERFKGKATLTSDTSSSTAYMQLSSLTSEDSAVYFCARSW |
| | | NYDARWGYWGQGTTLTVSS |
| 5D9 VL | 126 | EIVLTQSPASLAVSLGQRATISCRASESIDSYGISFIHWYQQKPGQPPKL |
| | | LIYRASNLESGIPARFSGSGSRTDFTLTINPVEAEDVATYYCQQSNEDPF |
| | | TFGSGTKLEIK |
| 606 VH | 127 | EVQLQQSGPELVKPGASVKISCKSSGYSFINFYIHWVKQRPGQGLDWIGW |
| | | IFPGSGETNFNEKFKVKAALTADTSSNTAYMQLSSLTSEDSAVYFCARSW |
| | | NYDARWGYWGQGTTVTVSS |
| 606 VL | 128 | QIVLTQTPASLAVSLGQRATISCRASESIDSYGISFMHWYQQKPGQPPKL |
| | | LIYRASNLESGIPARFSGSGSRPDFTLTINPVEADDVATYYCQQSNEDPF |
| | | TFGSGTKLEIK |
| 2D8 VH | 129 | QVQLKESGPGLVAPSQSLSITCTVSGFSLTSYGVSWVRQPPGKGLEWLGI |
| | | IWGDGSTNYHSALVSRLSISKDNSKSQVFLKLNSLQTDDTATYYCAKPNW |
| | | DYYAMDYWGQGTSVTVSS |
| 2D8 VL | 130 | DAVMTQTPASLAVSLGQRATISCRASESVDSYGISFMHWYQQKPGQPPKL |
| | | LIYRASILESGIPARFSGSGSRPDFSLTINPVEADDVATYYCQQSNEDPF |
| | | TFGSGTKLEIK |
| 48F12 VH | 131 | DVQLQESGPELVKPGASVKISCKSSGYSFINFYIHWVKQRPGQGLDWIGW |
| | | IFPGTGETNFNEKFKVKAALTADTSSSTVYMQLSTLTSEDSAVYFCARSW |
| | | NYDARWGYWGQGTSITVSS |
| 48F12 VL | 132 | DIQMTQTTSSLSASLGDRVTISCRASQDISNYLNWYQQKVDGTVKLLISY |
| | | TSRLHSGVPSRFSGSGSGTDYSLTISNLEQEDIATYFCQQGITLPLTFGA |
| | | GTKLELK |
| 1A9 VH | 133 | EVKLQQSGPDLVKPGASVKISCKASGYSFINFYIHWVKQRPGQGLEWIGW |
| | | IFPGNGETNYSEKFKGKATLTADTSSSTAYMQFNSLTYEDSAVYFCARTW |
| | | NYDARWGYWGQGTTLTVS |
| 1A9 VL | 134 | DVVMTQTPASLAVSLGQRATISCRASDGIDSYGISFMRWYQQKPGQPPTL |
| | | LIYRASTLESGIPARFSGSGSRTNFTLTINPVEADDVATYYCQQTNEDPF |
| | | TFGSGT KLEIK |
| 1E4C VH | 135 | QRELQQSGPELVKPGASVNISCKASGYSFTNHYINWVKQRPGQGLEWIGW |
| | | IFPGNGDTNYNEKFKGKATLTADTSSSTAYMQLSSLTSEDSAVYFCARTW |
| | | NYDARWGYWGQGTTVTVSS |
| 1E4C VL | 136 | DVVMTQTPAFLAVSLGQRATISCRASESIDSYGISFMHWYQQKPGQPPKV |
| | | LIYRTSNLESGIPARFSGSGSRTDFTLTINPVEADDVATYYCQQSNEDPF |
| | | TFGSGTKLEIK |
| 3A7A VH | 137 | QVQLKESGPELVKPGTSVKISCKASGYNFRNYYIQWVKQRPGQGLEWIGW |
| | | IFPGNNETNYNEKFKGKATLSADTSSTTAYMQLSSLTSEDSAVYFCARSW |
| | | NYDARWGYWGQGTTVTVSS |
| 3A7A VL | 138 | DVVMTQTPASLAVSLGQRATISCRASEIIDNYGISFIHWYQQKPGQPPKL |
| | | LIYRASNLESGIPARFSGSGSRTDSTLTINPVGADDVATYYCQQSNEDPF |
| | | TFGSGTKLELK |

In one embodiment, the anti-ILT2 antibodies can be prepared such that they do not have substantial specific binding to human Fcγ receptors, e.g., any one or more of CD16A, CD16B, CD32A, CD32B and/or CD64). Such antibodies may comprise constant regions of various heavy chains that are known to lack or have low binding to Fcγ receptors. Alternatively, antibody fragments that do not comprise (or comprise portions of) constant regions, such as F(ab′)2 fragments, can be used to avoid Fc receptor binding. Fc receptor binding can be assessed according to methods known in the art, including for example testing binding of an antibody to Fc receptor protein in a BIACORE assay. Also, generally any antibody IgG isotype can be used in which the Fc portion is modified (e.g., by introducing 1, 2, 3, 4, 5 or more amino acid substitutions) to minimize or eliminate binding to Fc receptors (see, e.g., WO 03/101485, the disclosure of which is herein incorporated by reference). Assays such as cell based assays, to assess Fc receptor binding are well known in the art, and are described in, e.g., WO 03/101485.

In one embodiment, the antibody can comprise one or more specific mutations in the Fc region that result in antibodies that have minimal interaction with effector cells. Reduced or abolished effector functions can be obtained by mutation in the Fc region of the antibodies and have been described in the art: N297A mutation, the LALA mutations, (Strohl, W., 2009, Curr. Opin. Biotechnol. vol. 20(6):685-691); and D265A (Baudino et al., 2008, J. Immunol. 181: 6664-69) see also Heusser et al., WO2012/065950, the disclosures of which are incorporated herein by reference. In one embodiment, an antibody comprises one, two, three or more amino acid substitutions in the hinge region. In one embodiment, the antibody is an IgG1 or IgG2 and comprises one, two or three substitutions at residues 233-236, optionally 233-238 (EU numbering). In one embodiment, the antibody is an IgG4 and comprises one, two or three substitutions at residues 327, 330 and/or 331 (EU numbering). Examples of modified Fc IgG1 antibodies that have reduced FcgammaR interaction are the LALA mutant comprising L234A and L235A mutation in the IgG1 Fc amino acid sequence. Another example of an Fc-reduced mutation is a mutation at residue D265, or at D265 and P329 for example as used in an IgG1 antibody as the DAPA (D265A, P329A) mutation (U.S. Pat. No. 6,737,056). Another modified IgG1 antibody comprises a mutation at residue N297 (e.g., N297A, N297S mutation), which results in aglycosylated/non-glycosylated antibodies. Other mutations include: substitutions at residues L234 and G237 (L234A/G237A); substitutions at residues S228, L235 and R409 (S228P/L235E/R409K,T,M,L); substitutions at residues H268, V309, A330 and A331 (H268Q/V309L/A330S/A331S); substitutions at residues C220, C226, C229 and P238 (C220S/C226S/C229S/P238S); substitutions at residues C226, C229, E233, L234 and L235 (C226S/C229S/E233P/L234V/L235A; substitutions at residues K322, L235 and L235 (K322A/L234A/L235A); substitutions at residues L234, L235 and P331 (L234F/L235E/P331S); substitutions at residues 234, 235 and 297; substitutions at residues E318, K320 and K322 (L235E/E318A/K320A/K322A); substitutions at residues (V234A, G237A, P238S); substitutions at residues 243 and 264; substitutions at residues 297 and 299; substitutions such that residues 233, 234, 235, 237, and 238 defined by the EU numbering system, comprise a sequence selected from PAAAP, PAAAS and SAAAS (see WO2011/066501).

In one embodiment, an antibody comprises a heavy chain constant region comprising the amino acid sequence below, or an amino acid sequence at least 90%, 95% or 99% identical thereto but retaining the amino acid residues at Kabat positions 234, 235 and 331 (underlined):

(SEQ ID NO: 42)
ASTKGPSVFPLAPSSKSTSGGTAALGCLVKDYFP
EPVTVSWNSGALTSGVHTFPAVLQSSGLYSLSSV
VTVPSSSLGTQTYICNVNHKPSNTKVDKRVEPKS
CDKTHTCPPCPAPEAEGGPSVFLFPPKPKDTLMI
SRTPEVICVVVDVSHEDPEVKFNWYVDGVEVHNA
KTKPREEQYNSTYRVVSVLTVLHQDWLNGKEYKC
KVSNKALPASIEKTISKAKGQPREPQVYTLPPSR
EEMTKNQVSLTCLVKGFYPSDIAVEWESNGQPEN
NYKTIPPVLDSDGSFFLYSKLTVDKSRWQQGNVF
SCSVMHEALHNHYTQKSLSLSPGK.

In one embodiment, an antibody comprises a heavy chain constant region comprising the amino acid sequence below, or an amino acid sequence at least 90%, 95% or 99% identical thereto but retaining the amino acid residues at Kabat positions 234, 235 and 331 (underlined):

(SEQ ID NO: 43)
ASTKGPSVFPLAPSSKSTSGGTAALGCLVKDYFP
EPVTVSWNSGALTSGVHTFPAVLQSSGLYSLSSV
VTVPSSSLGTQTYICNVNHKPSNTKVDKRVEPKS
CDKTHTCPPCPAPEFEGGPSVFLFPPKPKDTLMI
SRTPEVICVVVDVSHEDPEVKFNWYVDGVEVHNA
KTKPREEQYNSTYRVVSVLTVLHQDWLNGKEYKC
KVSNKALPASIEKTISKAKGQPREPQVYTLPPSR
EEMTKNQVSLTCLVKGFYPSDIAVEWESNGQPEN
NYKTIPPVLDSDGSFFLYSKLTVDKSRWQQGNVF
SCSVMHEALHNHYTQKSLSLSPGK.

In one embodiment, an antibody comprises a heavy chain constant region comprising the amino acid sequence below, or an amino acid sequence at least 90%, 95% or 99% identical thereto but retaining the amino acid residues at Kabat positions 234, 235, 237, 330 and 331 (underlined):

(SEQ ID NO: 44)
ASTKGPSVFPLAPSSKSTSGGTAALGCLVKDYFP
EPVTVSWNSGALTSGVHTFPAVLQSSGLYSLSSV
VTVPSSSLGTQTYICNVNHKPSNTKVDKRVEPKS
CDKTHTCPPCPAPEAEGAPSVFLFPPKPKDTLMI
SRTPEVTCVVVDVSHEDPEVKFNWYVDGVEVHNA
KTKPREEQYNSTYRVVSVLTVLHQDWLNGKEYKC
KVSNKALPSSIEKTISKAKGQPREPQVYTLPPSR
EEMTKNQVSLTCLVKGFYPSDIAVEWESNGQPEN
NYKTIPPVLDSDGSFFLYSKLTVDKSRWQQGNVF
SCSVMHEALHNHYTQKSLSLSPGK.

In one embodiment, an antibody comprises a heavy chain constant region comprising the amino acid sequence below, or a sequence at least 90%, 95% or 99% identical thereto but retaining the amino acid residues at Kabat positions 234, 235, 237 and 331 (underlined):

(SEQ ID NO: 45)
ASTKGPSVFPLAPSSKSTSGGTAALGCLVKDYFP
EPVTVSWNSGALTSGVHTFPAVLQSSGLYSLSSV
VTVPSSSLGTQTYICNVNHKPSNTKVDKRVEPKS
CDKTHTCPPCPAPEAEGAPSVFLFPPKPKDTLMI
SRTPEVTCVVVDVSHEDPEVKFNWYVDGVEVHNA
KTKPREEQYNSTYRVVSVLTVLHQDWLNGKEYKC
KVSNKALPASIEKTISKAKGQPREPQVYTLPPSR
EEMTKNQVSLTCLVKGFYPSDIAVEWESNGQPEN
NYKTIPPVLDSDGSFFLYSKLTVDKSRWQQGNVF
SCSVMHEALHNHYTQKSLSLSPGK.

Fragments and derivatives of antibodies (which are encompassed by the term “antibody” or “antibodies” as used in this application, unless otherwise stated or clearly contradicted by context) can be produced by techniques that are known in the art. “Fragments” comprise a portion of the intact antibody, generally the antigen binding site or variable region. Examples of antibody fragments include Fab, Fab′, Fab′-SH, F (ab′) 2, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv molecules (2) single chain polypeptides containing only one light chain variable domain, or a fragment thereof that contains the three CDRs of the light chain variable domain, without an associated heavy chain moiety and (3) single chain polypeptides containing only one heavy chain variable region, or a fragment thereof containing the three CDRs of the heavy chain variable region, without an associated light chain moiety; and multispecific (e.g., bispecific) antibodies formed from antibody fragments. Included, inter alia, are a nanobody, domain antibody, single domain antibody or a “dAb”.

In certain embodiments, the DNA of a hybridoma producing an antibody, can be modified prior to insertion into an expression vector, for example, by substituting the coding sequence for human heavy- and light-chain constant domains in place of the homologous non-human sequences (e.g., Morrison et al., PNAS pp. 6851 (1984)), or by covalently joining to the immunoglobulin coding sequence all or part of the coding sequence for a non-immunoglobulin polypeptide. In that manner, “chimeric” or “hybrid” antibodies are prepared that have the binding specificity of the original antibody. Typically, such non-immunoglobulin polypeptides are substituted for the constant domains of an antibody.

Optionally an antibody is humanized. “Humanized” forms of antibodies are specific chimeric immunoglobulins, immunoglobulin chains or fragments thereof (such as Fv, Fab, Fab′, F (ab′) 2, or other antigen-binding subsequences of antibodies) which contain minimal sequence derived from the murine immunoglobulin. For the most part, humanized antibodies are human immunoglobulins (recipient antibody) in which residues from a complementary-determining region (CDR) of the recipient are replaced by residues from a CDR of the original antibody (donor antibody) while maintaining the desired specificity, affinity, and capacity of the original antibody.

In some instances, Fv framework residues of the human immunoglobulin may be replaced by corresponding non-human residues. Furthermore, humanized antibodies can comprise residues that are not found in either the recipient antibody or in the imported CDR or framework sequences. These modifications are made to further refine and optimize antibody performance. In general, the humanized antibody will comprise substantially all of at least one, and typically two, variable domains, in which all or substantially all of the CDR regions correspond to those of the original antibody and all or substantially all of the FR regions are those of a human immunoglobulin consensus sequence. The humanized antibody optimally also will comprise at least a portion of an immunoglobulin constant region (Fc), typically that of a human immunoglobulin. For further details see Jones et al., Nature, 321, pp. 522 (1986); Reichmann et al, Nature, 332, pp. 323 (1988); Presta, Curr. Op. Struct. Biol., 2, pp. 593 (1992); Verhoeyen et Science, 239, pp. 1534; and U.S. Pat. No. 4,816,567, the entire disclosures of which are herein incorporated by reference.) Methods for humanizing the antibodies are well known in the art.

The choice of human variable domains, both light and heavy, to be used in making the humanized antibodies is very important to reduce antigenicity. According to the so-called “best-fit” method, the sequence of the variable domain of an antibody is screened against the entire library of known human variable-domain sequences. The human sequence which is closest to that of the mouse is then accepted as the human framework (FR) for the humanized antibody (Sims et al., J. Immunol. 151, pp. 2296 (1993); Chothia and Lesk, J. Mol. 196, 1987, pp. 901). Another method uses a particular framework from the consensus sequence of all human antibodies of a particular subgroup of light or heavy chains. The same framework can be used for several different humanized antibodies (Carter et al., PNAS 89, pp. 4285 (1992); Presta et al., J. Immunol., 151, p. 2623 (1993)).

It is further important that antibodies be humanized with retention of high affinity for ILT-2 receptors and other favorable biological properties. To achieve this goal, according to one method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequences so that the desired antibody characteristic, such as increased affinity for the target antigen (s), is achieved. In general, the CDR residues are directly and most substantially involved in influencing antigen binding.

Another method of making “humanized” monoclonal antibodies is to use a XenoMouse (Abgenix, Fremont, Calif.) as the mouse used for immunization. A XenoMouse is a murine host according that has had its immunoglobulin genes replaced by functional human immunoglobulin genes. Thus, antibodies produced by this mouse or in hybridomas made from the B cells of this mouse, are already humanized. The XenoMouse is described in U.S. Pat. No. 6,162,963, which is herein incorporated in its entirety by reference.

Human antibodies may also be produced according to various other techniques, such as by using, for immunization, other transgenic animals that have been engineered to express a human antibody repertoire (Jakobovitz et al., Nature 362 (1993) 255), or by selection of antibody repertoires using phage display methods. Such techniques are known to the skilled person and can be implemented starting from monoclonal antibodies as disclosed in the present application.

Compositions and Kits

Also provided herein are pharmaceutical compositions comprising a EGFR-binding antibody and/or an ILT-2 neutralizing agent such as an anti-ILT-2 antibody. In particular, in one aspect, provided is a pharmaceutical composition containing a neutralizing anti-EGFR antibody and a neutralizing anti-ILT-2 antibody, and optionally further a pharmaceutically acceptable carrier.

An anti-EGFR antibody and/or an ILT-2-neutralizing antibody can be incorporated in a pharmaceutical formulation in a concentration from 1 mg/ml to 500 mg/ml, wherein said formulation has a pH from 2.0 to 10.0.

The anti-EGFR antibody and the anti-ILT-2 agent can be comprised in the same or separate pharmaceutical formulations.

The formulation may further comprise a buffer system, preservative(s), tonicity agent(s), chelating agent(s), stabilizers and surfactants. In one embodiment, the pharmaceutical formulation is an aqueous formulation, i.e., formulation comprising water. Such formulation is typically a solution or a suspension. In a further embodiment, the pharmaceutical formulation is an aqueous solution. The term “aqueous formulation” is defined as a formulation comprising at least 50% w/w water. Likewise, the term “aqueous solution” is defined as a solution comprising at least 50% w/w water, and the term “aqueous suspension” is defined as a suspension comprising at least 50% w/w water.

In another embodiment, the pharmaceutical formulation is a freeze-dried formulation, whereto the physician or the patient adds solvents and/or diluents prior to use.

In another embodiment, the pharmaceutical formulation is a dried formulation (e.g. freeze-dried or spray-dried) ready for use without any prior dissolution.

In a further aspect, the pharmaceutical formulation comprises an aqueous solution of such an antibody, and a buffer, wherein the antibody is present in a concentration from 1 mg/ml or above, and wherein said formulation has a pH from about 2.0 to about 10.0.

In a another embodiment, the pH of the formulation is in the range selected from the list consisting of from about 2.0 to about 10.0, about 3.0 to about 9.0, about 4.0 to about 8.5, about 5.0 to about 8.0, and about 5.5 to about 7.5.

In a further embodiment, the buffer is selected from the group consisting of sodium acetate, sodium carbonate, citrate, glycylglycine, histidine, glycine, lysine, arginine, sodium dihydrogen phosphate, disodium hydrogen phosphate, sodium phosphate, and tris(hydroxymethyl)-aminomethan, bicine, tricine, malic acid, succinate, maleic acid, fumaric acid, tartaric acid, aspartic acid or mixtures thereof. Each one of these specific buffers constitutes an alternative embodiment of the invention.

In a further embodiment, the formulation further comprises a pharmaceutically acceptable preservative. In a further embodiment, the formulation further comprises an isotonic agent. In a further embodiment, the formulation also comprises a chelating agent. In a further embodiment of the invention the formulation further comprises a stabilizer. In a further embodiment, the formulation further comprises a surfactant. For convenience reference is made to Remington: The Science and Practice of Pharmacy, 19^{th} edition, 1995.

It is possible that other ingredients may be present in the pharmaceutical formulation of the present invention. Such additional ingredients may include wetting agents, emulsifiers, antioxidants, bulking agents, tonicity modifiers, chelating agents, metal ions, oleaginous vehicles, proteins (e.g., human serum albumin, gelatine or proteins) and a zwitterion (e.g., an amino acid such as betaine, taurine, arginine, glycine, lysine and histidine). Such additional ingredients, of course, should not adversely affect the overall stability of the pharmaceutical formulation of the present invention.

Administration of pharmaceutical compositions according to the invention may be through any appropriate route of administration, for example, intravenous. Suitable antibody formulations can also be determined by examining experiences with other already developed therapeutic monoclonal antibodies.

Also provided are kits, for example kits which include:
(i) a pharmaceutical composition containing an anti-EGFR antibody, and an ILT-2 neutralizing agent such as an anti-ILT-2 antibody, or
(ii) a first pharmaceutical composition containing an ILT-2 neutralizing agent such as an anti-ILT-2 antibody, and a second pharmaceutical composition containing an anti-EGFR antibody, or
(iii) a pharmaceutical composition containing antibody, and a second pharmaceutical composition containing an anti-EGFR antibody, and instructions to administer said anti-EGFR antibody with an ILT-2 neutralizing agent such as an anti-ILT-2 antibody, or
(iv) a pharmaceutical composition containing an ILT-2 neutralizing agent such as an anti-ILT-2 antibody, and instructions to administer said ILT-2 neutralizing agent antibody with an anti-EGFR antibody.

A pharmaceutical composition may optionally be specified as comprising a pharmaceutically-acceptable carrier. An anti-EGFR or anti-ILT-2 antibody may optionally be specified as being present in a therapeutically effective amount adapted for use in any of the methods herein. The kits optionally also can include instructions, e.g., comprising administration schedules, to allow a practitioner (e.g., a physician, nurse, or patient) to administer the composition contained therein to a patient having cancer. In any embodiment, a kit optionally can include instructions to administer said an anti-EGFR antibody simultaneously, separately, or sequentially with said anti-ILT-2 antibody. In any embodiment, a kit optionally can include instructions for use in the treatment of a cancer (e.g. a cancer further described herein). In any embodiment, a kit optionally can include instructions for use in the treatment of a colorectal cancer, for example. The kit also can include a syringe.

Optionally, the kits include multiple packages of the single-dose pharmaceutical compositions each containing an effective amount of the anti-EGFR antibody, and/or the anti-ILT-2 antibody, for a single administration in accordance with the methods provided above. Instruments or devices necessary for administering the pharmaceutical composition(s) also may be included in the kits. For instance, a kit may provide one or more pre-filled syringes containing an amount of the anti-EGFR or an anti-ILT-2 antibody.

### Diagnostics, Prognostics, and Treatment of Malignancies

Described are methods useful in the diagnosis, prognosis, monitoring and treatment of a HNSCC cancer in an individual. Described are methods useful in the diagnosis, prognosis, monitoring, treatment and prevention of a head and neck cancer in an individual. A HNSCC is a squamous cell or basaloid tumor that arises in the head or neck region and includes tumors of the nasal cavity, sinuses, lips, mouth and oral cavity, salivary glands, pharynx, or larynx. Anti-ILT-2 agents can be particularly useful for example in the treatment of oropharyngeal tumors, tumors of the larynx, tumors of the oral cavity and tumors of the hypopharynx. Such tumors are routinely identified by practitioners in the field of oncology, such as physicians, medical oncologists, histopathologists and oncologic clinicians. Treatment of HNSCC also includes the treatment of a premalignant lesion thereof. The premalignant lesions of HNSCC may include for example, dysplasia, hyperplasia, leukoplakia, erythroplakia, or hairy tongue. The methods can be for enhancing and/or eliciting an anti-tumor immune response in an individual. The methods can be for enhancing and/or potentiating the activity (e.g. cytotoxic activity toward cancer cells) of NK and/or CD8 T cells (optionally tumor-infiltrating NK and/or CD8 T cells) in an individual having HNSCC. Optionally, the anti-tumor immune response is at least partially mediated by NK and/or CD8 T cells. In another embodiment, the methods can be for enhancing and/or potentiating the anti-tumor immune response mediated by an antibody that binds EGFR (e.g. cetuximab).

In one embodiment, a tumor or cancer is known to be characterized by lack of or low HLA-A and/or HLA-G-expression, for example as assessed by detecting HLA-A- and/or HLA-G-expressing tumor cells, e.g., by immunohistochemistry. In one embodiment, a tumor cancer is known to be characterized by HLA-E-expression

In one embodiment, provided is use of an ILT-2-neutralizing antibody in combination with an anti-EGFR antibody. In one embodiment, provided is use of an ILT-2-neutralizing antibody in combination with an antibody that neutralizes the inhibitory activity of PD-1 as described herein, to advantageously treat a HNSCC.

In one aspect, a HLA-G-positive cancer is of a type or has a profile known to be generally or regularly characterized by lack or low levels of HLA-A (e.g. HLA-A2) and/or HLA-G-expression, for example at the surface of tumor cells). Accordingly, there is no requirement for a step of testing individuals or biological samples from individuals. In another aspect, HLA-G- and/or HLA-A2-expressing tumor cells can be detected in the tumor or tumor environment in order to determine if tumor or cancer is HLA-G and/or HLA-A2 positive or negative. In one embodiment, the HLA-G- and/or HLA-A2-negative cancer is characterized by a tumor determined (e.g. by in vitro detection of HLA-G and/or HLA-A2 in a tumor biopsy) to substantially lack HLA-G- and/or HLA-A2-expressing cells. In one aspect, the combination of an ILT-2-neutralizing antibody and an anti-EGFR antibody are used to treat an individual having an HLA-G- and HLA-A2-negative tumor or cancer. In one aspect, the combination of an ILT-2-neutralizing antibody and an anti-EGFR antibody are used to treat an individual having an HLA-G-negative tumor or cancer. In one aspect, the combination of an ILT-2-neutralizing antibody and an anti-EGFR antibody are used to treat an individual having an HLA-A2-negative tumor or cancer. In one aspect, the combination of an ILT-2-neutralizing antibody and an anti-EGFR antibody are used to treat an individual having a HLA-E-positive., HLA-G- and/or HLA-A2-negative tumor or cancer. In one aspect, the combination of an ILT-2-neutralizing antibody and an anti-EGFR antibody are used to treat a population of individuals that comprises (or that can comprise) individuals having an HLA-A2-negative tumor or cancer and/or individuals having an HLA-G-negative tumor or cancer.

Determining whether an individual has a cancer characterized by cells that express HLA-G and/or HLA-A2 polypeptides can for example comprise obtaining a biological sample (e.g. by performing a biopsy) from the individual that comprises cells from the cancer environment (e.g. tumor or tumor adjacent tissue), bringing said cells into contact with an antibody that binds an HLA-G polypeptide and/or an antibody that binds an HLA-A2 polypeptide, and detecting whether the cells express HLA-G and/or HLA-A2 on their surface. Optionally, determining whether an individual has cells that express HLA-G and/or HLA-A2 comprises conducting an immunohistochemistry assay.

As used herein, adjunctive or combined administration (co-administration) includes simultaneous administration of the compounds in the same or different dosage form, or separate administration of the compounds (e.g., sequential administration). Thus, an anti-EGFR antibody can be used in combination with the ILT-2 neutralizing antibody. For example, an anti-EGFR antibody and an anti-ILT2 antibody can be simultaneously administered in a single formulation. Alternatively, the anti-EGFR antibody and anti-ILT-2 antibody can be formulated for separate administration and are administered concurrently or sequentially.

Unless indicated otherwise, any of the treatment regimens and methods described herein may be used with or without a prior step of detecting the expression of HLA molecules on cells in a biological sample obtained from an individual (e.g. a biological sample comprising cancer cells, cancer tissue or cancer-adjacent tissue). In one embodiment, the cancer treated with the methods disclosed herein is a cancer characterized by HLA-E. In one embodiment, a cancer is a tumor or cancer known to be generally characterized by presence of HLA-E-expressing cells.

In another embodiment, the treatment regimens and methods described herein that combine ILT2-neutralizing antibodies and the anti-EGFR antibodies can be advantageously used in further combination with an agent that neutralizes the inhibitory activity of human PD-1, e.g., that inhibits the interaction between PD-1 and PD-L1. Examples of agents or antibodies that neutralize the inhibitory activity of human PD-1 include antibodies that bind PD1 or PD-L1. Many such antibodies are known and can be used, for example, at the exemplary the doses and/or frequencies that such agents are typically used. In one embodiment, the second or additional second therapeutic agent is an agent (e.g., an antibody) that inhibits the PD-1 axis (i.e. inhibits PD-1 or PD-L1).

PD-1 is an inhibitory member of the CD28 family of receptors that also includes CD28, CTLA-4, ICOS and BTLA. PD-1 is expressed on activated B cells, T cells, and myeloid cells Okazaki et al. (2002) Curr. Opin. Immunol. 14: 391779-82; Bennett et al. (2003) J Immunol 170:711-8). Two ligands for PD-1 have been identified, PD-L1 and PD-L2, that have been shown to downregulate T cell activation upon binding to PD-1 (Freeman et al. (2000) J Exp Med 192:1027-34; Latchman et al. (2001) Nat Immunol 2:261-8; Carter et al. (2002) Eur J Immunol 32:634-43). PD-L1 is abundant in a variety of human cancers (Dong et al. (2002) Nat. Med. 8:787-9). The interaction between PD-1 and PD-L1 results in a decrease in tumor infiltrating lymphocytes, a decrease in T-cell receptor mediated proliferation, and immune evasion by the cancerous cells. Immune suppression can be reversed by inhibiting the local interaction of PD-1 with PD-L1, and the effect is additive when the interaction of PD-1 with PD-L2 is blocked as well. Blockade of PD-1 can advantageously involve use of an antibody that prevents PD-L1-induced PD-1 signaling, e.g. by blocking the interaction with its natural ligand PD-L1. In one aspect the antibody binds PD-1 (an anti-PD-1 antibody); such antibody may block the interaction between PD-1 and PD-L1 and/or between PD-1 and PD-L2. In another aspect the antibody binds PD-L1 (an anti-PD-L1 antibody) and blocks the interaction between PD-1 and PD-L1.

There are currently at least six agents blocking the PD-1/PD-L1 pathway that are marketed or in clinical evaluation, any of these may be useful in combination with the anti-ILT2 antibodies of the disclosure. One agent is BMS-936558 (Nivolumab/ONO-4538, Bristol-Myers Squibb; formerly MDX-1106). Nivolumab, (Trade name Opdivo®) is an FDA-approved fully human IgG4 anti-PD-L1 mAb that inhibits the binding of the PD-L1 ligand to both PD-1 and CD80 and is described as antibody 5C4 in WO 2006/121168, the disclosure of which is incorporated herein by reference. For melanoma patients, the most significant OR was observed at a dose of 3 mg/kg, while for other cancer types it was at 10 mg/kg. Nivolumab is generally dosed at 10 mg/kg every 3 weeks until cancer progression. Another agent is durvalumab (Imfinzi®, MEDI-4736), an anti-PD-L1 developed by AstraZeneca/Medimmune and described in WO2011/066389 and US2013/034559. Another agent is MK-3475 (human IgG4 anti-PD1 mAb from Merck), also referred to as lambrolizumab or pembrolizumab (Trade name Keytruda®) has been approved by the FDA for the treatment of melanoma and is being tested in other cancers. Pembrolizumab was tested at 2 mg/kg or 10 mg/kg every 2 or 3 weeks until disease progression. Another agent is atezolizumab (Tecentriq®, MPDL3280A/RG7446, Roche/Genentech), a human anti-PD-L1 mAb that contains an engineered Fc domain designed to optimize efficacy and safety by minimizing FcγR binding and consequential antibody-dependent cellular cytotoxicity (ADCC). Doses of ≤1, 10, 15, and 25 mg/kg MPDL3280A were administered every 3 weeks for up to 1 year. In phase 3 trial, MPDL3280A is administered at 1200 mg by intravenous infusion every three weeks in NSCLC. In other aspects, a treatment or use may optionally be specified as not being in combination with (or excluding treatment with) an antibody or other agent that inhibits the PD-1 axis.

The present disclosure also provides an agent that is an antibody that binds to ILT-2 and neutralizes the inhibitory activity of ILT-2 in an NK cell, for use in treating a human individual who has cancer, wherein said antibody that binds ILT-2 is administered in combination with an anti-EGFR antibody.

For instance, also provided are:

the agent for use as described above, wherein said individual has a HNSCC, optionally a metastatic and/or recurrent HNSCC;

the agent for use as described above, wherein said anti-EGFR antibody is an antibody that inhibits EGFR;

the agent for use as described above, wherein said ILT-2 neutralizing agent is an antibody that binds a human ILT-2 protein, optionally a human or humanized anti-ILT-2 antibody;

the agent for use as described above, wherein said ILT-2-neutralizing agent is an antibody that is capable of inhibiting the binding of ILT-2 to HLA-G1;

the agent for use as described above, wherein said ILT-2-neutralizing agent comprises (a) the heavy chain H-CDR1, H-CDR2 and H-CDR3 domains having the sequences of SEQ ID NOS: 14-16, and the light chain L-CDR1, L-CDR2 and L-CDR3 domains having the sequences of SEQ ID NOS: 17-19, respectively; or (b) the heavy chain H-CDR1, H-CDR2 and H-CDR3 domains having the sequences of SEQ ID NOS: 22-24, and the light chain L-CDR1, L-CDR2 and L-CDR3 domains having the sequences of SEQ ID NOS: 25-27, respectively; or (c) the heavy chain H-CDR1, H-CDR2 and H-CDR3 domains having the sequences of SEQ ID NOS: 30-32, and the light chain L-CDR1, L-CDR2 and L-CDR3 domains having the sequences of SEQ ID NOS: 33-35, respectively;

the agent for use as described above, wherein said anti-EGFR antibody and said antibody that binds ILT-2 are administered simultaneously, separately, or sequentially;

the agent for use as described above, wherein said anti-EGFR antibody and said antibody that binds ILT-2 are formulated for separate administration and are administered concurrently or sequentially; and/or

the agent for use as described above, wherein said anti-EGFR antibody is administered at a dose ranging from 0.1 to 10 mg/kg and said antibody that binds ILT-2 is administered at a dose ranging from 1 to 20 mg/kg. In one embodiment, an ILT-2-neutralizing antibody can be administered in an amount that induces or increases immune cell (e.g. CD8 T cell, NK cell) infiltration into a tumor.

In the combination treatment methods, when anti-EGFR antibody is administered in combination with an ILT-2-neutralizing antibody, the anti-EGFR antibody and ILT-2-neutralizing antibody can be administered separately, together or sequentially, or in a cocktail. In some embodiments, the anti-EGFR antibody is administered prior to the administration of the ILT-2-neutralizing antibody. For example, the anti-EGFR antibody can be administered approximately 0 to 30 days prior to the administration of the ILT-2-neutralizing antibody. In some embodiments, the anti-EGFR antibody is administered from about 30 minutes to about 2 weeks, from about 30 minutes to about 1 week, from about 1 hour to about 2 hours, from about 2 hours to about 4 hours, from about 4 hours to about 6 hours, from about 6 hours to about 8 hours, from about 8 hours to 1 day, or from about 1 to 5 days prior to the administration of the anti-ILT-2 antibodies. In some embodiments, an anti-EGFR antibody s administered concurrently with the administration of the ILT-2-neutralizing antibody. In some embodiments, an anti-EGFR antibody is administered after the administration of the ILT-2-neutralizing antibody. For example, an anti-EGFR antibody can be administered approximately 0 to 30 days after the administration of the ILT-2-neutralizing antibody. In some embodiments, an anti-EGFR antibody is administered from about 30 minutes to about 2 weeks, from about 30 minutes to about 1 week, from about 1 hour to about 2 hours, from about 2 hours to about 4 hours, from about 4 hours to about 6 hours, from about 6 hours to about 8 hours, from about 8 hours to 1 day, or from about 1 to 5 days after the administration of the ILT-2-neutralizing antibody.

### EXAMPLES

### Example 1: ILT2 (LILRB1) is Expressed on Healthy Human Donor Memory CD8 T Cells and CD56dim NK Cells

LILRB1 expression on peripheral blood mononuclear cells was determined by flow cytometry on fresh whole blood from healthy human donors. The NK population was determined as CD3−CD56+ cells (anti CD3 AF700—BioLegend #300424; anti CD56 BV421—BD Biosciences #740076). Among NK cells, CD56bright subset was identify as CD16− cells whereas CD56dim subset as CD16+ cells (anti CD16 BV650—BD Biosciences #563691). CD4+ and CD8+ T cells were identify as CD3+CD56−CD4+ and CD3+CD56−CD8+ cells, respectively (CD3—see above; CD4 BV510—BD Biosciences #740161; CD8 BUV737—BD Biosciences #564629). Among the CD4+ T cell population, Tconv and Treg were identify as CD127+CD25−/low and CD127lowCD25high cells, respectively (CD127 PE-Cy7—BD Biosciences #560822; CD25 VioBright—Miltenyi Biotec #130-104-274). Among the CD8+ T cell population, the naïve, central memory, effector memory and effector memory T cell populations were identify as CD45RA+CCR7+, CD45RA−CCR7+, CD45RA−CCR7−, CD45RA+CCR7− cells, respectively (CD45RA BUV395—BD Biosciences #740298; CCR7 PerCP-Cy5.5—BioLegend #353220). A population named “CD3+CD56+ ly” was an heterogeneous cell population comprising NKT cells and γδ T cells. Monocytes were identify as CD3−CD56−CD14+ cells (CD14 BV786—BD Biosciences #563691) and B cells as CD3−CD56−CD19+ cells (CD19 BUV496—BD Biosciences #564655). Anti-LILRB1 antibody (clone HP-F1—APC—BioLegend #17-5129-42) as used. Whole blood was incubated 20 min at RT in the dark with staining Ab mix then red blood cells were lyzed with Optilyse C (Beckman Coulter #A11895) following the provider TDS. Cells were washed twice with PBS and fluorescence was revealed with Fortessa flow cytometer (BD Biosciences).

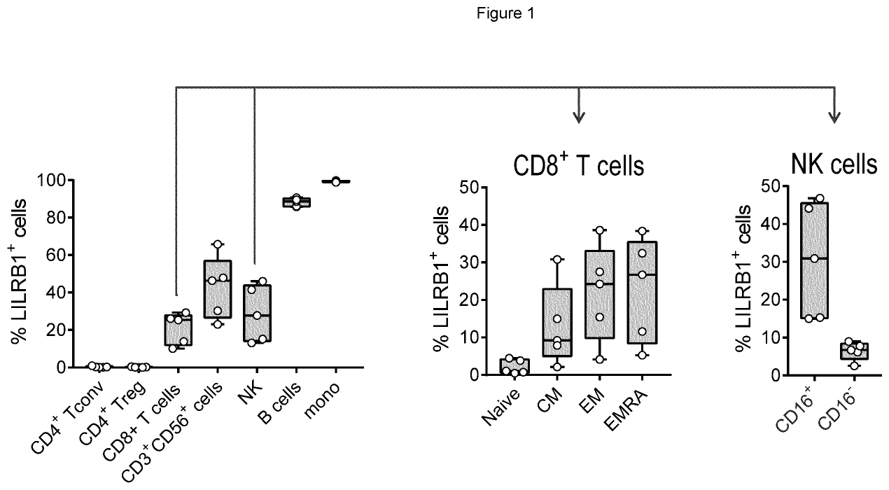

Results are shown in FIG. 1. While B lymphocytes and monocytes generally always express ILT2, conventional CD4 T cells and CD4 Treg cells did not express ILT2, but a significant fraction of CD8 T cells (about 25%), CD3+ CD56+ lymphocytes (about 50%) and NK cells (about 30%) expressed ILT2, suggesting that a proportion of each of such CD8 T and NK cell populations can be inhibited by ILT2, as a function of the HLA class I ligands present, for example on tumor cells.

Among the CD8 T cells, ILT2 expression was not present on naïve cells, but was present in effector memory fraction of CD8 T cells, and to a lesser extent, central memory CD8 T cells. Among the NK cells, the ILT2 expression was essentially only on the CD16+ subset (CD56dim), and much less frequently on CD16− NK cells (CD56bright).

### Example 2: ILT2 is Upregulated in Multiple Human Cancers

ILT2 expression on monocytes, B cells, CD4+ T cells, CD8+ T cells and both CD16− and CD16+ NK cells was determined by flow cytometry on peripheral blood mononuclear cells (PBMC) purified from whole blood of human cancer patient donors. Cell populations were identified and ILT2 expression was assessed using the same antibody mix detailed in example 1. PBMC were incubated 20 min at 4° C. in the dark with the antibody mix, wash twice in staining buffer and fluorescence was measured on a Fortessa flow cytometer.

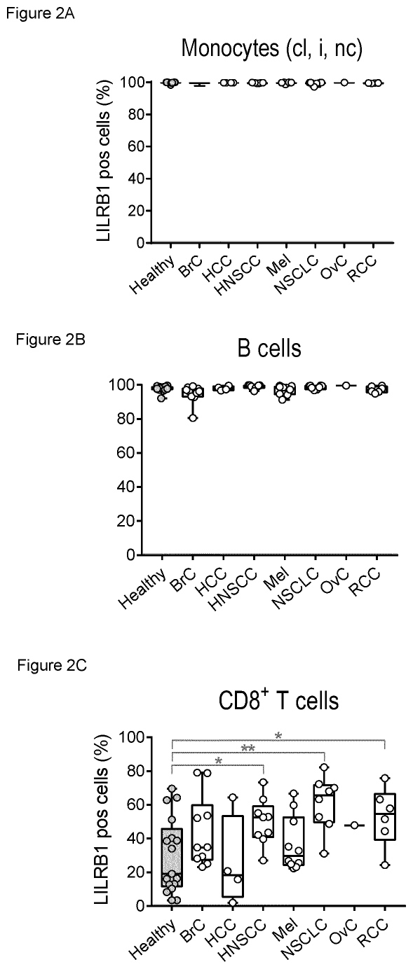

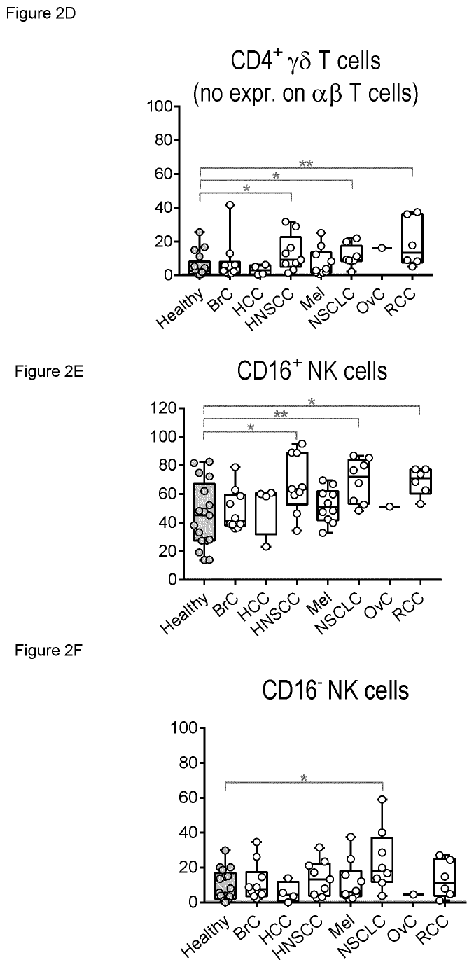

Results from the cancer patient samples are shown in FIG. 2. As can be seen, ILT2 was once again expressed on all monocytes and B cells. However on the lymphocyte subsets, NK cells and CD8 T cells, ILT2 was expressed more frequently with statistical significance on cells from three types of cancers, HNSCC, NSCLC and RCC. ILT2 was upregulated also in ovarian cancer although greater numbers of patient samples need to be studied. This increased expression of ILT2 in cancer patient samples was observed in CD8 T cells, γδ T cells (no expression on αβ T cells) and CD16+ NK cells, in head and neck cancer (HNSCC), lung cancer (NSCLC) and kidney cancer (RCC).

### Example 3: Generation of Anti-ILT2 Antibodies

### Materials and Methods

### Cloning and Production of the ILT-2_6×His Recombinant Protein

The ILT-2 protein (Uniprot access number Q8NHL6) was cloned into the pTT-5 vector between the NruI and BamHI restriction sites. A heavy chain peptide leader was used. The PCR were performed with the following primers:

(SEQ ID NO: 57)
ILT-2_For_ACAGGCGTGCATTCGGGGCACCTCCCCAAGCCCAC,
(SEQ ID NO: 58)
ILT-2_Rev_CGAGGTCGGGGGATCCTCAATGGTGGTGATGATGGT
GGTGCCTTCCCAGACCACTCTG,

A 6×His tag was added at the C-terminal part of the protein for purification. The EXP1293 cell line was transfected with the generated vector for transient production. The protein was purified from the supernantant using Ni-NTA beads and monomers were purified using a SEC.

The amino acid sequence for the ILT-2_6×His recombinant protein is shown below:

(SEQ ID NO: 59)
GHLPKPTLWAEPGSVITQGSPVTLRCQGGQETQEYRLYREKKTALWITR
IPQELVKKGQFPIPSITWEHAGRYRCYYGSDTAGRSESSDPLELVVTGA
YIKPTLSAQPSPVVNSGGNVILQCDSQVAFDGFSLCKEGEDEHPQCLNS
QPHARGSSRAIFSVGPVSPSRRWWYRCYAYDSNSPYEWSLPSDLLELLV
LGVSKKPSLSVQPGPIVAPEETLTLQCGSDAGYNRFVLYKDGERDFLQL
AGAQPQAGLSQANFTLGPVSRSYGGQYRCYGAHNLSSEWSAPSDPLDIL
IAGQFYDRVSLSVQPGPTVASGENVTLLCQSQGWMQTFLLTKEGAADDP
WRLRSTYQSQKYQAEFPMGPVTSAHAGTYRCYGSQSSKPYLLTHPSDPL
ELVVSGPSGGPSSPTTGPTSTSGPEDQPLITTGSDPQSGLGRHHHHHHH

### Generation of CHO and KHYG Cell Lines Expressing ILT Family Members at the Cell Surface

The complete forms of ILT-2 were amplified by PCR using the following primers: ILT-2_For ACAGGCGTGCATTCGGGGCACCTCCCCAAGCCC (SEQ ID NO: 60), and ILT-2_Rev_CCGCCCCGACTCTAGACTAGTGGATGGCCAGAGTGG (SEQ ID NO: 61). The PCR products were inserted into the expression vector at appropriate restriction sites. A heavy chain peptide leader was used. The vectors were then transfected into the CHO and KHYG cell lines to obtain stable clones expressing the ILT-2 protein at the cell surface. These cells were then used for hybridoma screening. CHO cells expressing other ILT family members were prepared similarly, including cells expressing ILT-1, ILT-3, ILT-4, ILT-5, ILT-6, ILT7 and ILT-8. The amino acid sequences of the ILT proteins used to prepare the ILT-1, ILT-3, ILT-4, ILT-5 and ILT-6-expressing cells are provided in Table 4 below.

### Generation of K562 Cell Line Expressing HLA-G at the Cell Surface

The complete forms of HLA-G (Genbank access number NP_002118.1, sequence shown below) was amplified by PCR using the following primers: HLA-G_For 5′ CCAGAACACAGGATCCGCCGCCACCATGGTGGTCATGGCGCCC 3′ (SEQ ID NO: 62), HLA-G_Rev_5′ TTTTCTAGGTCTCGAGTCAATCTGAGCTCTTCTTTC 3′ (SEQ ID NO: 63). The PCR products were inserted into a vector between the BamHI and XhoI restriction sites and used to transduce K562 cell lines which either did not express HLA-E or were engineered to stably overexpress HLA-E.

```
HLA-G amino acid sequence:
(SEQ ID NO: 10)
    1 MVVMAPRTLF LLLSGALTLT ETWAGSHSMR YFSAAVSRPG RGEPRFIAMG YVDDTQFVRF
  61 DSDSACPRME PRAPWVEQEG PEYWEEETRN TKAHAQTDRM NLQTLRGYYN QSEASSHTLQ
121 WMIGCDLGSD GRLLRGYEQY AYDGKDYLAL NEDLRSWTAA DTAAQISKRK CEAANVAEQR
181 RAYLEGTCVE WLHRYLENGK EMLQRADPPK THVTHHPVFD YEATLRCWAL GFYPAEIILT
241 WQRDGEDQTQ DVELVETRPA GDGTFQKWAA VVVPSGEEQR YTCHVQHEGL PEPLMLRWKQ
301 SSLPTIPIMG IVAGLVVLAA VVTGAAVAAV LWRKKSSD
HLA-E amino acid sequence (Uniprot P13747):
(SEQ ID NO: 11)
MVDGTLLLLL SEALALTQTW AGSHSLKYFH TSVSRPGRGE PRFISVGYVD
DTQFVRFDND AASPRMVPRA PWMEQEGSEY WDRETRSARD TAQIFRVNLR
TLRGYYNQSE AGSHTLQWMH GCELGPDGRF LRGYEQFAYD GKDYLTLNED
LRSWTAVDTA AQISEQKSND ASEAEHQRAY LEDTCVEWLH KYLEKGKETL
LHLEPPKTHV THHPISDHEA TLRCWALGFY PAEITLTWQQ DGEGHTQDTE
LVETRPAGDG TFQKWAAVVV PSGEEQRYTC HVQHEGLPEP VTLRWKPASQ
PTIPIVGIIA GLVLLGSVVS GAVVAAVIWR KKSSGGKGGS YSKAEWSDSA
QGSESHSL
```

### Immunization and Screening

An immunization was performed by immunizing balb/c mice with ILT-2_6×His protein. After the immunization protocol the mice were sacrificed to perform fusions and get hybridomas. The hybridoma supernatants were used to stain CHO-ILT2 and CHO-ILT4 cell lines to check for monoclonal antibody reactivities in a flow cytometry experiment. Briefly, the cells were incubated with 50 μl of supernatant for 1H at 4° C., washed three times and a secondary antibody Goat anti-mouse IgG Fc specific antibody coupled to AF647 was used (Jackson Immunoresearch, JI115-606-071). After 30 min of staining, the cells were washed three times and analyzed using a FACS CANTO II (Becton Dickinson).

About 1500 hybridoma supernatants were screened, to identify those producing antibodies that bind to ILT2 and have the ability to block the interaction between ILT2 with HLA-G. Briefly, recombinant 6×HIS tagged ILT2 was incubated with 50 μl of hybridoma supernatant for 20 min at RT prior incubation with 10⁵ K562 cells expressing HLA-G. Then, cells were washed once and incubated with a secondary complex made of rabbit anti-6×HIS (Bethyl lab, A190-214A) antibody and anti-rabbit IgG F(ab′)² antibody coupled to PE (Jackson lab, 111-116-114). After 30 min of staining, the cells were washed once in PBS and fixed with Cell Fix (Becton Dickinson, 340181). Analysis was performed on a FACS CANTO II flow cytometer.

This assays permitted the identification of a panel of anti-ILT2 antibodies that were highly effective in blocking the interaction of ILT2 with its HLA class I ligand HLA-G. Antibodies 3H5, 12D12, 26D8, 18E1, 27C10, 27H5, 1C11, 1D6, 9G1, 19F10a and 27G10 were identified as having good blocking activity and thus selected for further study.

The resulting antibodies were produced as modified human IgG1 antibodies having heavy chains with Fc domain mutations L234A/L235E/G237A/A330S/P331S (Kabat EU numbering) which resulted in lack of binding to human Fcγ receptors CD16A, CD16B, CD32A, CD32B and CD64. These Fc domain mutated L234A/L235E/G237A/A330S/P331S antibodies were then used in all the other experiments described herein. Briefly, the VH and Vk sequences of each antibody (the VH and Vk variable regions shown in herein) were cloned into expression vectors containing the huIgG1 constant domains harboring the aforementioned mutations and the huCk constant domain respectively. The two obtained vectors were co-transfected into the CHO cell line. The established pool of cell was used to produce the antibody in the CHO medium.

### Example 4: Binding of Modified Human IgG1 Fc Domains to FcγR

The L234A/L235E/G237A/A330S/P331S Fc domains employed in Example 3, as well as other Fc mutations and wild-type antibodies, were previously evaluated to assess binding to human Fcγ receptors, as follows.

SPR (Surface Plasmon Resonance) measurements were performed on a Biacore T100 apparatus (Biacore GE Healthcare) at 25° C. In all Biacore experiments HBS-EP+ (Biacore GE Healthcare) and 10 mM NaOH, 500 mM NaCl served as running buffer and regeneration buffer respectively. Sensorgrams were analyzed with Biacore T100 Evaluation software. Recombinant human FcR's (CD64, CD32a, CD32b, CD16a and CD16b) were cloned, produced and purified.

Antibodies tested included: antibodies having wild type human IgG1 domain, antibodies having a human IgG4 domain with S241P substitution, human IgG1 antibodies having a N297S substitution, human IgG1 antibodies having L234F/L235E/P331S substitutions, human IgG1 antibodies having L234A/L235E/P331S substitutions, human IgG1 antibodies having L234A/L235E/G237A/A330S/P331S substitutions, and human IgG1 antibodies having L234A/L235E/G237A/P331S substitutions.

Antibodies were immobilized covalently to carboxyl groups in the dextran layer on a Sensor Chip CMS. The chip surface was activated with EDC/NHS (N-ethyl-N′-(3-dimethylaminopropyl) carbodiimidehydrochloride and N-hydroxysuccinimide (Biacore GE Healthcare)). Antibodies were diluted to 10 μg/ml in coupling buffer (10 mM acetate, pH 5.6) and injected until the appropriate immobilization level was reached (i.e. 800 to 900 RU). Deactivation of the remaining activated groups was performed using 100 mM ethanolamine pH 8 (Biacore GE Healthcare).

Monovalent affinity study was assessed following a classical kinetic wizard (as recommended by the manufacturer). Serial dilutions of soluble analytes (FcRs) ranging from 0.7 to 60 nM for CD64 and from 60 to 5000 nM for all the other FcRs were injected over the immobilized bispecific antibodies and allowed to dissociate for 10 min before regeneration. The entire sensorgram sets were fitted using the 1:1 kinetic binding model for CD64 and with the Steady State Affinity model for all the other FcRs.

The results are shown in Table 7, below. Results showed that while full length wild type human IgG1 bound to all human Fcγ receptors, and human IgG4 in particular bound significantly to FcγRI (CD64) (KD shown in Table 7), the L234A/L235E/G237A/A330S/P331S substitutions and L234A/L235E/G237A/P331S substitutions abolished binding to CD64 as well as to CD16a.

### Example 5: Ability of ILT2 Blocking Antibodies to Enhance NK Cell Lysis

The ability of the anti-ILT2 antibodies to control ILT2-mediated inhibition of NK cell activation was determined by the capacity of ILT2-expressing KHYG cells described in Example 3 to lyse target cells in presence of antibodies. Effector cells were KHYG cells expressing ILT2 and GFP as control and target cells were ⁵¹Cr loaded K562 cell line (ATCC® CCL-243™) made to express HLA-G. Effector and target cells were mixed at a ratio 1:10. Antibodies were pre-incubated 30 minutes at 37° C. with effector cells and then target cells were co-incubated 4 hours at 37° C. Specific lysis of target cells was calculated by the release of ⁵¹Cr in co-culture supernatant with a TopCount NXT (Perkin Elmer).

This experiment evaluated antibodies 3H5, 12D12, 26D8, 18E1, 27C10, 27H5, 1C11, 1D6, 9G1, 19F10a, 27G10 identified in Example 2, as well as commercially available antibodies GHI/75 (mouse IgG2b, Biolegend #333720), 292319 (mouse IgG2b, Bio-Techne #MAB20172), HP-F1 (mouse IgG1, eBioscience #16-5129-82), 586326 (mouse IgG2b, Bio-Techne #MAB30851) and 292305 (mouse IgG1, Bio-Techne #MAB20171).

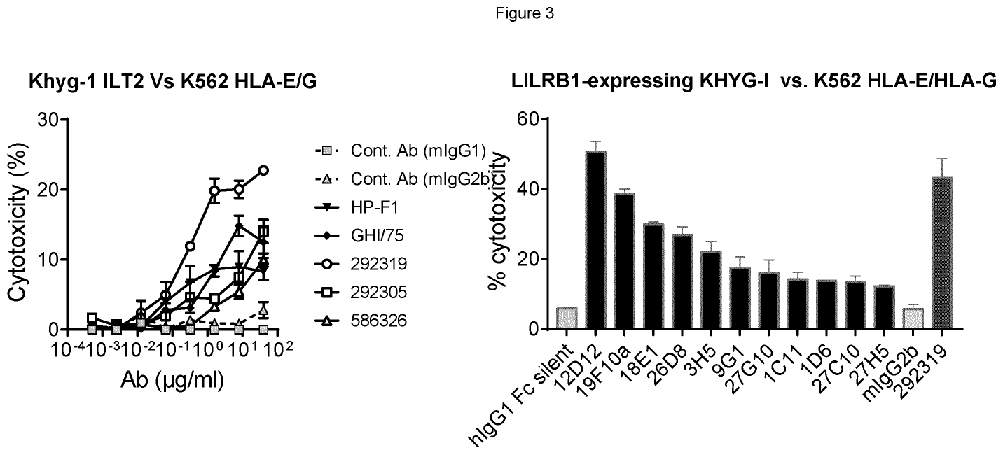

Results are shown in FIG. 3. Most of the ILT2/HLA-G blocking antibodies showed a significant increase in % cytotoxicity by the NK cell lines toward the K562-HLA-G tumor target cells. However, certain antibodies were particular potent at increasing NK cell cytotoxicity. Antibodies 12D12, 19F10a and commercial 292319 were significantly more effective than other antibodies in the ability to enhance NK cell cytotoxicity toward the target cells. Antibodies 18E1, 26D8, although less effective, displayed activity as enhancers of cytotoxicity, followed to a lesser extent by 3H5 and commercial antibody HP-F1. Other antibodies, including 27C10, 27H5, 1C11, 1D6, 9G1 and commercial antibodies 292305, 586326, GHI/75 were considerably less active than 18E1, 26D8 in their ability to induce cytotoxicity toward target cells.

### Example 6: Blockade of ILT2 Binding to HLA Class I Molecules

HLA/ILT2 Blocking Assay

Ability of anti-ILT2 antibodies to block the interactions between HLA-G or HLA-A2 expressed at the surface of cell lines and recombinant ILT2 protein was assessed by flow cytometry. Briefly, BirA-tagged ILT2 protein was biotinylated to obtain 1 biotin molecule per ILT2 protein. APC-conjugated streptavidin (SA) was mixed with Biotinylated ILT2 protein (ratio 1 Streptavidin per 4 ILT2 protein) to form tetramers. Anti-ILT2 Abs (12D12, 18E1, 26D8) were incubated at 4° C. in staining buffer for 30 min with ILT2-SA tetramers. The Ab-ILT2-SA complexes were added on HLA-G or HLA-A2 expressing cells and incubated for 1 hour at 4° C. The binding of complexes on cells was evaluated on a Accury C6 flow cytometer equipped with an HTFC plate loader and analyzed using the FlowJo software.

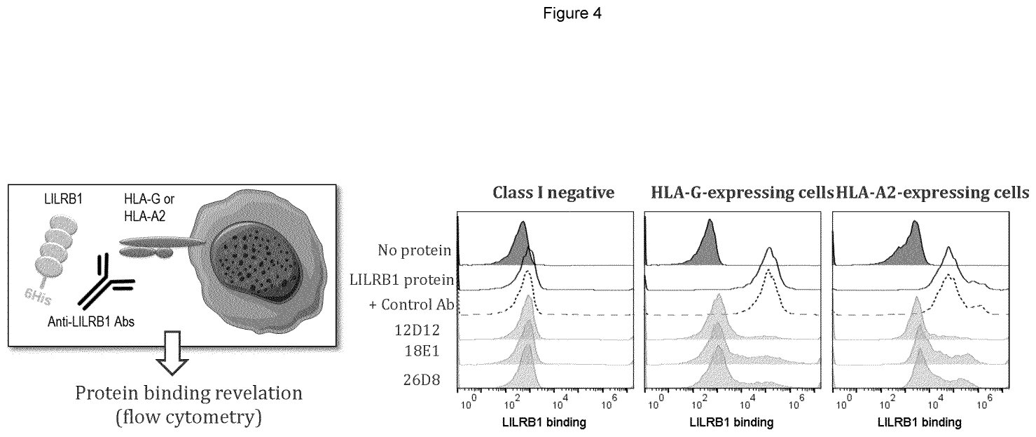

This assays permitted the identification of a panel of anti-ILT2 antibodies that were highly effective in blocking the interaction of ILT2 with its HLA class I ligand HLA-G. Antibodies 3H5, 12D12, 26D8, 18E1, 27C10, 27H5, 1C11, 1D6, 9G1, 19F10a and 27G10 all blocked ILT2 binding to HLA-G and HLA-A2. FIG. 4 shows representative results for antibodies 12D12, 18E1, and 26D8.

### Example 7: Antibody Titration on ILT2-Expressing Cells by Flow Cytometry

In order to explain the differences in NK cytotoxicity induction, unlabeled antibodies 3H5, 12D12, 26D8, 18E1, 27C10, 27H5, 1C11, 1D6, 9G1, 19F10a and 27G10 as well as the commercially available antibodies GHI/75, 292319, HP-F1, 586326 and 292305 were tested in experiments for binding to CHO cells modified to express human ILT-2. Cells were incubated with various concentrations of unlabeled anti-ILT2 antibodies from 30 μg/ml to 5×10-⁴ μg/ml, for 30 minutes at 4° C. After washes with staining buffer, cells were incubated for 30 min at 4° C. with Goat anti-human H+L AF488 secondary antibody (Jackson Immunoresearch #109-546-088) or Goat anti-mouse H+L AF488 secondary antibody for commercially available antibodies (Jackson Immuoresearch #115-545-146). Fluorescence was measured on an Accury C6 flow cytometer equipped with an HTFC plate loader.

Results are shown in Table 1, below. Except for antibody GHI/75 which had an EC50 in the range of 1-log higher that the other antibodies, the rest of the antibodies all showed comparable EC50 values, suggesting that differences binding affinity does not explain the observed differences in ability to enhance NK cell cytotoxicity.

TABLE 1

| | CHO-ILT2 | Primary NK |
|---|---|---|
| | cells | cells |
| Antibody | EC50 (μg/mL) | EC50 (μg/mL) |
| 3H5 | 0.35 | 0.48 |
| 12D12 | 0.36 | 0.09 |
| 26D8 | 0.15 | 0.11 |
| 18E1 | 0.12 | 0.11 |
| 27C10 | 0.25 | 0.33 |
| 27H5 | 0.52 | NA |
| 1C11 | 0.30 | 0.22 |
| 1D6 | 0.21 | 0.20 |
| 9G1 | 0.35 | 0.24 |
| 19F10a | 0.11 | 0.09 |
| 27G10 | 0.21 | 1.1 |
| HP-F1 | 0.56 | 0.09 |
| 292319 | 0.22 | 0.47 |
| 586326 | 0.13 | ND |
| GHI/75 | 5.39 | ND |
| 292305 | 0.27 | ND |

### Example 8: Monovalent Affinity Determination

Antibodies 3H5, 12D12, 26D8, 18E1, 27C10, 27H5, 1C11, 1D6, 9G1, 19F10a, and 27G10 as well as the commercially available antibodies GHI/75, 292319 and HP-F1 were tested for binding affinity to human ILT2 proteins.

SPR (Surface Plasmon Resonance) methods were used to test antibodies 3H5, 12D12, 26D8, 18E1, 27C10, 27H5, 1C11, 1D6, 9G1, 19F10a, 27G10 (all of human IgG1 isotype). Measurements were performed on a Biacore T200 apparatus (Biacore GE Healthcare) at 25° C. In all Biacore experiments HBS-EP+ (Biacore GE Healthcare) and NaOH 10 mM served as running buffer and regeneration buffer respectively. Sensorgrams were analyzed with Biacore T100 Evaluation software. Protein-A was purchased from (GE Healthcare). Human ILT2 recombinant proteins were cloned, produced and purified at Innate Pharma. Protein-A proteins were immobilized covalently to carboxyl groups in the dextran layer on a Sensor Chip CMS. The chip surface was activated with EDC/NHS (N-ethyl-N′-(3-dimethylaminopropyl) carbodiimidehydrochloride and N-hydroxysuccinimide (Biacore GE Healthcare)). Protein-A was diluted to 10 μg/ml in coupling buffer (10 mM acetate, pH 5.6) and injected until the appropriate immobilization level was reached (i.e. 600 RU). Deactivation of the remaining activated groups was performed using 100 mM ethanolamine pH 8 (Biacore GE Healthcare). Anti-ILT2 antibodies at 2 μg/mL were captured onto the Protein-A chip and recombinant human ILT2 proteins were injected at different concentrations in a range from 250 nM to 1.95 nM over captured antibodies. For blank subtraction, cycles were performed again replacing ILT2 proteins with running buffer. The monovalent affinity analysis was conducted following a regular Capture-Kinetic protocol as recommended by the manufacturer (Biacore GE Healthcare kinetic wizard). Seven serial dilutions of human ILT2 proteins, ranging from 1.95 nM to 250 nM were sequentially injected over the captured antibodies and allowed to dissociate for 10 min before regeneration. The entire sensorgram sets were fitted using the 1:1 kinetic binding model or two state reaction model, as a function of the profile of the curves.

OCTET analysis was used to evaluate antibodies GHI/75, 292319 and HP-F1, (all mouse isotypes). Measurements were performed on an Octet RED96 System (Fortebio). In all Biacore experiments Kinetics Buffer 10× (Fortebio) and Glycine 10 mM pH 1.8 served as running buffer and regeneration buffer respectively. Graphs were analyzed with Data Analysis 9.0 software. Anti-Mouse IgG Fc Capture (AMC) biosensors are used. Anti-ILT2 antibodies at 5 μg/mL were captured onto Anti-Mouse IgG Fc Capture (AMC) biosensors. Seven dilutions of recombinant human ILT2 proteins were injected (from 1000 nM to 15.625 nM for 292319 and HP-F1 and from 100 nM to 1.5625 nM for GHI-75). The curves were fitted using the model 1:1

Results are shown in Table 2, below. The KD differences generally does not appear to correlate to the differences in ability to enhance NK cell cytotoxicity. Binding affinity therefore does not explain the differences in the antibodies' ability to enhance NK cell cytotoxicity.

TABLE 2

| mAb | KD (nM) | Ka (1/ms) | Kd (1/s) |
|---|---|---|---|
| 3H5 | 4.4 | ka1: 2.8E+5 | kd1: 8.0E−3 |
| | | ka2: 8.7E−4 | kd2: 1.6E−4 |
| 12D12 | 1.0 | 4.3E+5 | 4.2E−4 |
| 26D8 | 0.4 | 6.2E+5 | 2.2E−4 |
| 18E1 | 0.2 | 7.5E+5 | 1.1E−4 |
| 27C10 | 0.2 | 1.4E+5 | 3.0E−4 |
| 27H5 | 13.9 | ka1: 6.6E+5 | kd1: 0.1 |
| | | ka2: 5.3E−3 | kd2: 4.2E−4 |
| 1C11 | 0.3 | 3.4E+5 | 1.1E−4 |
| 1D6 | 0.4 | 3.2E+5 | 1.2E−4 |
| 9G1 | 0.3 | 4.0E+5 | 1.3E−4 |
| 19F10a | 5.3 | 6.6E+5 | 3.5E−3 |
| 27G10 | 0.5 | 3.5E+5 | 1.8E−4 |
| GHI/75 | 28.1 | 1.3E+4 | 3.8E−4 |
| 292319 | 0.6 | 3.0E+5 | 1.7E−4 |
| HP-F1 | 2.3 | 4.6E+5 | 1.1E−3 |

### Example 9: Identification of Antibodies that Increase Cytotoxicity in Primary Human NK Cells

We considered the possibility that the inability of prior antibodies to neutralize ILT2 in NK cells might be related to differences in ILT2 expression in primary NK cells compared for example to highly selected or modified NK cell lines that express much higher levels of ILT2 at their surface. We studied and selected antibodies in primary NK cells from a number of healthy human donors. The effect of the anti-ILT2 antibodies of Example 5 was studied by activation assays by assessing CD137 surface expression on NK cells. In each case, primary NK cells (as fresh NK cells purified from donors) were used as effector cells and K562 cells (chronic myelogenous leukemia (CML)) expressing HLA-E/G were used as targets. The targets consequently thus expressed not only the ILT2 ligand HLA-G, but also HLA-E which is an HLA class I ligand expressed on the surface of a range of cancer cells and which can interact with inhibitory receptors on the surface of NK and CD8 T cells.

Briefly, the effect of the anti-ILT2 antibodies on NK cells activation was determined by analysis by flow cytometry of CD137 expression on total NK cells, ILT2-positive NK cells and ILT2-negative NK cells. Effector cells were primary NK cells (fresh NK cells purified from donors, incubation overnight at 37° C. before use) and target cells (K562 HLA-E/G cell line) were mixed at a ratio 1:1. The CD137 assay was carried out in 96 U well plates in completed RPMI, 200 μL final/well. Antibodies were pre-incubated 30 minutes at 37° C. with effector cells and then target cells were co-incubated overnight at 37° C. The following steps were: spin 3 min at 500 g; wash twice with Staining Buffer (SB); addition of 50 μL of staining Ab mix (anti-CD3 Pacific blue—BD Biosciences; anti-CD56-PE-Vio770—Miltenyi Biotec; anti-CD137-APC—Miltenyi Biotec; anti-ILT2-PE—clone HP-F1, eBioscience); incubation 30 min at 4° C.; wash twice with SB; resuspended pellet with SB; and fluorescence revealed with Canto II (HTS). Negative controls were NK cells vs K562-HLA-E/G alone and in presence of isotype control.

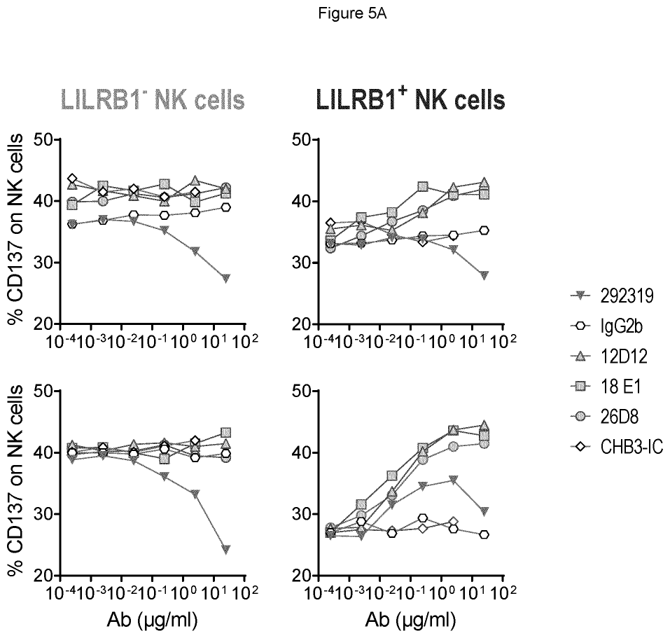

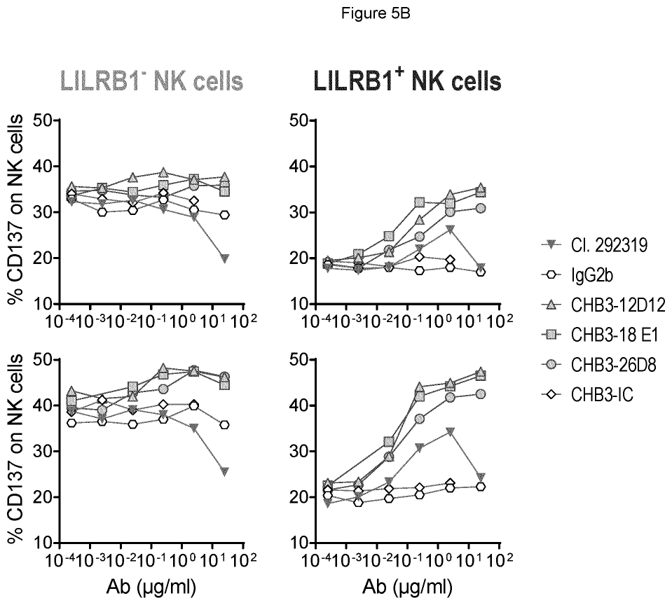

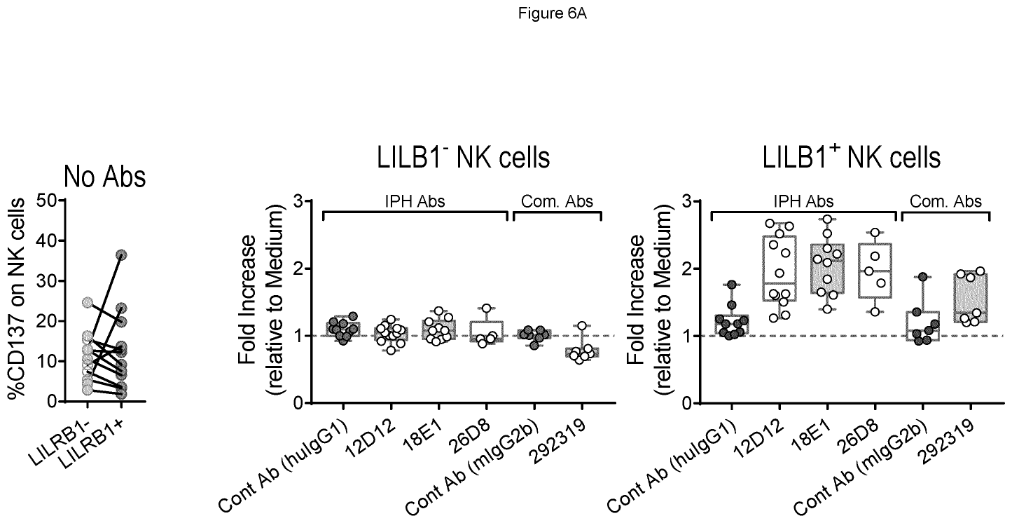

FIG. 5A is a representative figure showing the increase of % of total NK cells expressing CD137 mediated by anti-ILT2 antibodies using NK cells from two human donors and K562 tumor target cells made to express HLA-E and HLA-G. FIG. 5B is a representative figure showing the increase of % of ILT2-positive (left hand panel) and ILT2-negative (right hand panel) NK cells expressing CD137 mediated anti-ILT2 antibodies using NK cells from two human donors and an HLA-A2-expressing B cell line.

Surprisingly, it was observed that antibodies that were most effective in enhancing cytotoxicity of NK cell lines were not necessarily able to activate the primary human NK cells. Among the antibodies 12D12, 19F10a and 292319 that were most effective in enhancing cytotoxicity of NK cell lines, both 19F10a and 292319 substantially lacked the ability to activate the primary NK cells all, compared to isotype control antibodies.

On the other hand, antibodies 12D12, 18E1 and 26D8 showed strong activation of the primary NK cells. Study of ILT2-positive NK cells showed that these antibodies mediated a two-fold increase in activation of the NK cells toward the target cells. As a control, % of ILT2-negative NK cells expressing CD137 were not affected by the antibodies.

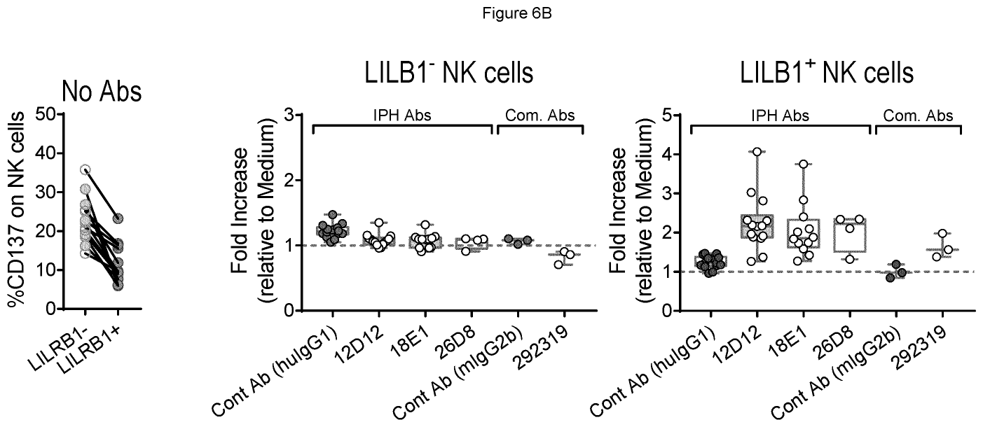

FIGS. 6A and 6B shows the ability of antibodies to enhance cytotoxicity of primary NK cells toward the tumor target cells in terms of fold-increase of cytotoxicity marker CD137. FIG. 6A shows the ability of antibodies to enhance NK cell activation in presence of HLA-G-expressing target cells using primary NK cells from 5-12 different donors against HLA-G and HLA-E expressing K562 target cells. FIG. 6A shows the ability of antibodies to enhance NK cell activation in presence of HLA-G-expressing target cells using primary NK cells from 3-14 different donors against the HLA-A2 expressing target B cells. In each case 12D12, 18E1 and 26D8 had greater enhancement of NK cytotoxicity compared to one of the antibodies (292319) which was among the antibodies showing strongest enhancement of NK cytotoxicity when using NK cell lines in Example 5.

### Example 10: Characterization of Binding to ILT Family Members

To further characterize the binding specificity of the antibodies, antibodies were tested by flow cytometry for binding to the cells made to express different ILT family proteins. In addition to ILT2 (LILRB1)-expressing cells described above, cells expressing human ILT1 (LILRA2), ILT3 (LILRB4), ILT4 (LILRB2), ILT5 (LILRB3), ILT6 (LILRA3), ILT7 (LILRA4) or ILT8 (LILRA6) were generated.

The human ILT genes were amplified by PCR using the primers described in Table 3 below. The PCR product were inserted into the expression vector at appropriate restriction sites. A heavy chain peptide leader was used and a V5 tag having the amino acid sequence GKPIPNPLLGLDST (SEQ ID NO: 80) was added at the N-terminal (not shown in the sequences in Table 4). Amino acid sequences for different human ILT proteins used herein are shown below in Table 4, below. The vectors were then transfected into the CHO cell line to obtain stable clones expressing the different ILT proteins at the cell surface.

TABLE 3

| | Genbank | |
|---|---|---|
| Constructs | number | Forward primers |
| ILT-1 | NM_001130917.2 | 5′ ACAGGCGTGCATTCGGGTAAGCCTATCCCTAACCCTCTCCTCG |
| | | GTCTCGATTCTACGGGGCACCTCCCCAAGCCCACCCTCTGGGCTGA |
| | | GCC 3′ (SEQ ID NO: 64) |
| ILT-2 | Q8NHL6.1 | 5′ ACAGGCGTGCATTCGGGTAAGCCTATCCCTAACCCTCTCCTCG |
| | | GTCTCGATTCTACGGGGCACCTCCCCAAGCCCACCCTCTGGGCTGA |
| | | GCC 3′ (SEQ ID NO: 65) |
| ILT-3 | NM_001278428.3 | 5′ ACAGGCGTGCATTCGGGTAAGCCTATCCCTAACCCTCTCCTCG |
| | | GTCTCGATTCTACGGGGCCCCTCCCCAAACCCACCCTCTGGGCTGA |
| | | GCCA 3′ (SEQ ID NO: 66) |
| ILT-4 | Q8N423.4 | 5′ ACAGGCGTGCATTCGGGTAAGCCTATCCCTAACCCTCTCCTCG |
| | | GTCTCGATTCTACGGGGACCATCCCCAAGCCCACCCTGTGGGCTGA |
| | | GCCA 3′ (SEQ ID NO: 67) |
| ILT-5 | AF000575.1 | 5′ ACAGGCGTGCATTCGGGTAAGCCTATCCCTAACCCTCTCCTCG |
| | | GTCTCGATTCTACGGGGCCCTTCCCCAAACCCACCCTCTGGGCTGA |
| | | GCC 3′ (SEQ ID NO: 68) |
| ILT-6 | | 5′ ACAGGCGTGCATTCGGGTAAGCCTATCCCTAACCCTCTCCTCG |
| | | GTCTCGATTCTACGGGGCCCCTCCCCAAACCCACCCTCTGGGCTGA |
| | | GCCA 3′ (SEQ ID NO: 69) |
| ILT-7 | AF041261.1 | 5′ ACAGGCGTGCATTCGGGTAAGCCTATCCCTAACCCTCTCCTCG |
| | | GTCTCGATTCTACGGAAAACCTACCCAAACCCATCCTGTGGGCCGA |
| | | GCCA 3′ (SEQ ID NO: 70) |
| ILT-8 | AF041262.1 | 5′ ACAGGCGTGCATTCGGGTAAGCCTATCCCTAACCCTCTCCTCG |
| | | GTCTCGATTCTACGGGGCCCTTCCCCAAACCCACCCTCTGGGCTGA |
| | | GCC 3′ (SEQ ID NO: 71) |
| ILT-1 | NM_001130917.2 | 5′ CCGCCCCGACTCTAGATCATCTCTGGCTGTGCTGAGC 3′ |
| | | (SEQ ID NO: 72) |
| ILT-2 | Q8NHL6.1 | 5′ CCGCCCCGACTCTAGACTAGTGGATGGCCAGAGTGG 3′ |
| | | (SEQ ID NO: 73) |
| ILT-3 | NM_001278428.3 | 5′ CCGCCCCGACTCTAGATCAGGCATAGACACTGGGCTC 3′ |
| | | (SEQ ID NO: 74) |
| ILT-4 | Q8N423.4 | 5′ CCGCCCCGACTCTAGACTAGTGGATGGCCAGGGTGG 3′ |
| | | (SEQ ID NO: 75) |
| ILT-5 | AF000575.1 | 5′ CCGCCCCGACTCTAGATCAGGCGTAGATGCTGGGCTC 3′ |
| | | (SEQ ID NO: 76) |
| ILT-6 | | 5′ CCGCCCCGACTCTAGATCAAGAGTAAAGATGCAGAAGACTAAG |
| | | ACTGACTACAAATAGGGAAGCAGTAGATTGAAGAGCACCCTCACCA |
| | | GCCTTGGAGTCGGACTTGTTTTGTGGT 3′ (SEQ ID NO: 77) |
| ILT-7 | AF041261.1 | 5′ CCGCCCCGACTCTAGATCACTCCACCACTCTGAAGGG 3′ |
| | | (SEQ ID NO: 78) |
| ILT-8 | AF041262.1 | 5′ CCGCCCCGACTCTAGATCAATCTTGGGGGTTTCTCTG 3′ |
| | | (SEQ ID NO: 79) |

TABLE 4
ILT sequences

| | SEQ ID | |
|---|---|---|
| Protein | NO | Sequence (AA) |
| Human | 3 | GHLPKPTLWAEPGSVIIQGSPVTLRCQGSLQAEEYHLYRENKSASWVRRIQEP |
| ILT-1 | | GKNGQFPIPSITWEHAGRYHCQYYSHNHSSEYSDPLELVVTGAYSKPTLSALP |
| | | SPVVTLGGNVTLQCVSQVAFDGFILCKEGEDEHPQRLNSHSHARGWSWAIFSV |
| | | GPVSPSRRWSYRCYAYDSNSPYVWSLPSDLLELLVPGVSKKPSLSVQPGPMVA |
| | | PGESLTLQCVSDVGYDRFVLYKEGERDFLQRPGWQPQAGLSQANFTLGPVSPS |
| | | HGGQYRCYSAHNLSSEWSAPSDPLDILITGQFYDRPSLSVQPVPTVAPGKNVT |
| | | LLCQSRGQFHTFLLTKEGAGHPPLHLRSEHQAQQNQAEFRMGPVTSAHVGTYR |
| | | CYSSLSSNPYLLSLPSDPLELVVSASLGQHPQDYTVENLIRMGVAGLVLVVLG |
| | | ILLFEAQHSQR |
| Human | 2 | GHLPKPTLWAEPGSVITQGSPVTLRCQGGQETQEYRLYREKKTAPWITRIPQE |
| ILT-2 | | LVKKGQFPIPSITWEHAGRYRCYYGSDTAGRSESSDPLELVVTGAYIKPTLSA |
| | | QPSPVVNSGGNVTLQCDSQVAFDGFILCKEGEDEHPQCLNSQPHARGSSRAIF |
| | | SVGPVSPSRRWWYRCYAYDSNSPYEWSLPSDLLELLVLGVSKKPSLSVQPGPI |
| | | VAPEETLTLQCGSDAGYNRFVLYKDGERDFLQLAGAQPQAGLSQANFTLGPVS |
| | | RSYGGQYRCYGAHNLSSEWSAPSDPLDILIAGQFYDRVSLSVQPGPTVASGEN |
| | | VTLLCQSQGWMQTFLLTKEGAADDPWRLRSTYQSQKYQAEFPMGPVTSAHAGT |
| | | YRCYGSQSSKPYLLTHPSDPLELVVSGPSGGPSSPTTGPTSTSAGPEDQPLTP |
| | | TGSDPQSGLGRHLGVVIGILVAVILLLLLLLLLFLILRHRRQGKHWTSTQRKA |
| | | DFQHPAGAVGPEPTDRGLQWRSSPAADAQEENLYAAVKHTQPEDGVEMDTRSP |
| | | HDEDPQAVTYAEVKHSRPRREMASPPSPLSGEFLDTKDRQAEEDRQMDTEAAA |
| | | SEAPQDVTYAQLHSLTLRRKATEPPPSQEGEPPAEPSIYATLAIH |
| Human | 4 | GPLPKPTLWAEPGSVISWGNSVTIWCQGTLEAREYRLDKEESPAPWDRQNPLE |
| ILT-3 | | PKNKARFSIPSMTEDYAGRYRCYYRSPVGWSQPSDPLELVMTGAYSKPTLSAL |
| | | PSPLVTSGKSVTLLCQSRSPMDTFLLIKERAAHPLLHLRSEHGAQQHQAEFPM |
| | | SPVTSVHGGTYRCFSSHGFSHYLLSHPSDPLELIVSGSLEGPRPSPTRSVSTA |
| | | GPEDQPLMPTGSVPHSGLRRHWEVLIGVLVVSILLLSLLLFLLLQHWRQGKHR |
| | | TLAQRQADFQRPPGAAEPEPKDGGLQRRSSPAADVQGENFCAAVKNTQPEDGV |
| | | EMDTRQSPHDEDPQAVTYAKVKHSRPRREMASPPSPLSGEFLDTKDRQAEEDR |
| | | QMDTEAAASEAPQDVTYAQLHSFTLRQKATEPPPSQEGASPAEPSVYA |
| Human | 5 | GTIPKPTLWAEPDSVITQGSPVTLSCQGSLEAQEYRLYREKKSASWITRIRPE |
| ILT-4 | | LVKNGQFHIPSITWEHTGRYGCQYYSRARWSELSDPLVLVMTGAYPKPTLSAQ |
| | | PSPVVISGGRVTLQCESQVAFGGFILCKEGEEEHPQCLNSQPHARGSSRAIFS |
| | | VGPVSPNRRWSHRCYGYDLNSPYVWSSPSDLLELLVPGVSKKPSLSVQPGPVV |
| | | APGESLTLQCVSDVGYDRFVLYKEGERDLRQLPGRQPQAGLSQANFTLGPVSR |
| | | SYGGQYRCYGAHNLSSECSAPSDPLDILITGQIRGTPFISVQPGPTVASGENV |
| | | TLLCQSWRQFHTFLLTKAGAADAPLRLRSIHEYPKYQAEFPMSPVTSAHAGTY |
| | | RCYGSLNSDPYLLSHPSEPLELVVSGPSMGSSPPPTGPISTPGPEDQPLTPTG |
| | | SDPQSGLGRHLGVVIGILVAVVLLLLLLLLLFLILRHRRQGKHWTSTQRKADF |
| | | QHPAGAVGPEPTDRGLQWRSSPAADAQEENLYAAVKDTQPEDGVEMDTRAAAS |
| | | EAPQDVTYAQLHSLTLRRKATEPPPSQEREPPAEPSIYATLAIH |
| Human | 6 | GPFPKPTLWAEPGSVISWGSPVTIWCQGSLEAQEYRLDKEGSPEPLDRNNPLE |
| ILT-5 | | PKNKARFSIPSMTEHHAGRYRCHYYSSAGWSEPSDPLELVMTGFYNKPTLSAL |
| | | PSPVVASGGNMTLRCGSQKGYHHFVLMKEGEHQLPRTLDSQQLHSGGFQALFP |
| | | VGPVNPSHRWRFTCYYYYMNTPQVWSHPSDPLEILPSGVSRKPSLLTLQGPVL |
| | | APGQSLTLQCGSDVGYDRFVLYKEGERDFLQRPGQQPQAGLSQANFTLGPVSP |
| | | SHGGQYRCYGAHNLSSEWSAPSDPLNILMAGQIYDTVSLSAQPGPTVASGENV |
| | | TLLCQSWWQFDTFLLTKEGAAHPPLRLRSMYGAHKYQAEFPMSPVTSAHAGTY |
| | | RCYGSYSSNPHLLSHPSEPLELVVSGHSGGSSLPPTGPPSTPGLGRYLEVLIG |
| | | VSVAFVLLLFLLLFLLLRRQRHSKHRTSDQRKTDFQRPAGAAETEPKDRGLLR |
| | | RSSPAADVQEENLYAAVKDTQSEDRVELDSQSPHDEDPQAVTYAPVKHSSPRR |
| | | EMASPPSSLSGEFLDTKDRQVEEDRQMDTEAAASEASQDVTYAQLHSLTLRRK |
| | | ATEPPPSQEGEPPAEPSIYA |
| Human | 7 | GPLPKPTLWAEPGSVITQGSPVTLRCQGSLETQEYHLYREKKTALWITRIPQE |
| ILT-6 | | LVKKGQFPILSITWEHAGRYCCIYGSHTAGLSESSDPLELVVTGAYSKPTLSA |
| | | LPSPVVTSGGNVTIQCDSQVAFDGFILCKEGEDEHPQCLNSHSHARGSSRAIF |
| | | SVGPVSPSRRWSYRCYGYDSRAPYVWSLPSDLLGLLVPGVSKKPSLSVQPGPV |
| | | VAPGEKLTFQCGSDAGYDRFVLYKEWGRDFLQRPGRQPQAGLSQANFTLGPVS |
| | | RSYGGQYTCSGAYNLSSEWSAPSDPLDILITGQIRARPFLSVRPGPTVASGEN |
| | | VTLLCQSQGGMHTFLLTKEGAADSPLRLKSKRQSHKYQAEFPMSPVTSAHAGT |
| | | YRCYGSLSSNPYLLTHPSDPLELVVSGAAETLSPPQNKSD |
| Human | 8 | ENLPKPILWAEPGPVITWHNPVTIWCQGTLEAQGYRLDKEGNSMSRHILKTLE |
| ILT-7 | | SENKVKLSIPSMMWEHAGRYHCYYQSPAGWSEPSDPLELVVTAYSRPTLSALP |
| | | SPVVTSGVNVTLRCASRLGLGRFTLIEEGDHRLSWTLNSHQHNHGKFQALFPM |
| | | GPLTFSNRGTFRCYGYENNTPYVWSEPSDPLQLLVSGVSRKPSLLTLQGPVVT |
| | | PGENLTLQCGSDVGYIRYTLYKEGADGLPQRPGRQPQAGLSQANFTLSPVSRS |
| | | YGGQYRCYGAHNVSSEWSAPSDPLDILIAGQISDRPSLSVQPGPTVTSGEKVT |
| | | LLCQSWDPMFTFLLTKEGAAHPPLRLRSMYGAHKYQAEFPMSPVTSAHAGTYR |
| | | CYGSRSSNPYLLSHPSEPLELVVSGATETLNPAQKKSDSKTAPHLQDYTVENL |
| | | IRMGVAGLVLLFLGILLFEAQHSQRSPPRCSQEANSRKDNAPFRVVE |
| Human | 9 | GPFPKPTLWAEPGSVISWGSPVTIWCQGSLEAQEYQLDKEGSPEPLDRNNPLE |
| ILT-8 | | PKNKARFSIPSMTQHHAGRYRCHYYSSAGWSEPSDPLELVMTGFYNKPTLSAL |
| | | PSPVVASGGNMTLRCGSQKGYHHFVLMKEGEHQLPRTLDSQQLHSGGFQALFP |
| | | VGPVTPSHRWRFTCYYYYTNTPRVWSHPSDPLEILPSGVSRKPSLLTLQGPVL |
| | | APGQSLTLQCGSDVGYDRFVLYKEGERDFLQRPGQQPQAGLSQANFTLGPVSP |
| | | SHGGQYRCYGAHNLSSEWSAPSDPLNILMAGQIYDTVSLSAQPGPTVASGENV |
| | | TLLCQSRGYFDTFLLTKEGAAHPPLRLRSMYGAHKYQAEFPMSPVTSAHAGTY |
| | | RCYGSYSSNPHLLSFPSEPLELMVSASHAKDYTVENLIRMGMAGLVLVFLGIL |
| | | LFEAQHSQRNPQD |

Briefly, for the flow cytometry screening, antibodies were incubated 1 hour with each ILT-expressing CHO cell lines (CHO ILT1 cell line, CHO ILT2 cell line, CHO ILT3 cell line, CHO ILT4 cell line, CHO ILT5 cell line, CHO ILT6 cell line, CHO ILT7 cell line, CHO ILT8 cell line), washed twice in staining buffer, revealed by Goat anti-mouse IgG H+L polyclonal antibody (pAb) labeled with PE (for commercially available antibodies, Jackson Immuoresearch #115-116-146) or Goat anti-human IgG H+L pAb labeled with PE (for chimeric antibodies, Jackson Immunoresearch #109-116-088) washed twice with staining buffer and stainings were acquired on a Accury C6 flowcytometer equipped with an HTFC plate loader and analyzed using the FlowJo software.

Results showed that many of the anti-ILT2 antibodies bound also to ILT6 (LILRA3) in addition to ILT2, either alone (i.e. ILT2/ILT6 cross-reactive) or with additional binding to ILT4 or ILT5 (i.e. ILT2/ILT4/ILT6 or ILT2/ILT5/ILT6 cross-reactive). Antibodies 1C11, 1D6, 9G1, 19F10a, 27G10, commercial antibodies 586326 and 292305 bound to ILT2 and also ILT6. Antibody 586326 furthermore also bound to ILT4 in addition to ILT2 and ILT6, whereas antibody 292305 further bound ILT5 in addition to ILT2 and ILT6. Finally, commercial antibody 292319 bound to ILT1 in addition to ILT2 (ILT1/ILT2 cross-reactive). However, a subset of antibodies exemplified by 3H5, 12D12, 26D8, 18E1, 27C10 and 27H5 bound only to ILT2 and no other ILT family member protein.

### Example 11: Epitope Mapping

### Anchored ILT2 Domain Fragment Proteins

### Generation of ILT2 Proteins

Nucleic acid sequences encoding different human ILT2 domains D1 (corresponding to residues 24-121 of the sequence shown in SEQ ID NO: 1), D2 (corresponding to residues 122-222 of the sequence shown in SEQ ID NO: 1), D3 (corresponding to residues 223-321 of the sequence shown in SEQ ID NO: 1), D4 (corresponding to residues 322-458 of the sequence shown in SEQ ID NO: 1), and combinations thereof, were amplified by PCR using the primers described in the Table below. The PCR products were inserted into an expression vector at appropriate restriction sites. A heavy chain peptide leader was used and a V5 tag was added at the N-terminal and expression at the surface of cells was confirmed by flow cytometry. For all of the domains that were not followed by a D4 domain, a CD24 GPI anchor was added to permit anchoring at the cell membrane. The amino acid sequences of the resulting different human ILT2 domain fragment-containing proteins are shown below in Table 5, below. The vectors were then transfected into the CHO cell line to obtain stable clones expressing the different ILT2 domain proteins at the cell surface.

TABLE 5

| | | SEQ |
|---|---|---|
| | | ID |
| Description | Amino acid sequence | NO |
| D1 domain | TGVHSGKPIPNPLLGLDSTGHLPKPTLWAEPGSVITQGSPVTLRCQGGQETQ | 46 |
| | EYRLYREKKTALWITRIPQELVKKGQFPIPSITWEHAGRYRCYYGSDTAGRS | |
| | ESSDPLELVVTGAGALQSTASLFVVSLSLLHLYS | |
| D2 domain | TGVHSGKPIPNPLLGLDSTYIKPTLSAQPSPVVNSGGNVILQCDSQVAFDGF | 47 |
| | SLCKEGEDEHPQCLNSQPHARGSSRAIFSVGPVSPSRRWWYRCYAYDSNSPY | |
| | EWSLPSDLLELLVLGVGALQSTASLFVVSLSLLHLYS | |
| D3 domain | TGVHSGKPIPNPLLGLDSTSKKPSLSVQPGPIVAPEETLTLQCGSDAGYNRF | 48 |
| | VLYKDGERDFLQLAGAQPQAGLSQANFTLGPVSRSYGGQYRCYGAHNLSSEW | |
| | SAPSDPLDILIAGQGALQSTASLFVVSLSLLHLYS | |
| D4 domain | TGVHSGKPIPNPLLGLDSTFYDRVSLSVQPGPTVASGENVTLLCQSQGWMQT | 49 |
| | FLLTKEGAADDPWRLRSTYQSQKYQAEFPMGPVTSAHAGTYRCYGSQSSKPY | |
| | LLTHPSDPLELVVSGPSGGPSSPTTGPTSTSGPEDQPLTPTGSDPQSGLGRH | |
| | LGVVIGILVAVILLLLLLLLLFLILRHRRQGKHWTSTQRKADFQHPAGAVGP | |
| | EPTDRGLQWRSSPAADAQEENLYAAVKHTQPEDGVEMDTRSPHDEDPQAVTY | |
| | AEVKHSRPRREMASPPSPLSGEFLDTKDRQAEEDRQMDTEAAASEAPQDVTY | |
| | AQLHSLTLRREATEPPPSQEGPSPAVPSIYATLAIH | |
| D1-D2 | TGVHSGKPIPNPLLGLDSTGHLPKPTLWAEPGSVITQGSPVTLRCQGGQETQ | 50 |
| domain | EYRLYREKKTALWITRIPQELVKKGQFPIPSITWEHAGRYRCYYGSDTAGRS | |
| | ESSDPLELVVTGAYIKPTLSAQPSPVVNSGGNVILQCDSQVAFDGFSLCKEG | |
| | EDEHPQCLNSQPHARGSSRAIFSVGPVSPSRRWWYRCYAYDSNSPYEWSLPS | |
| | DLLELLVLGVGALQSTASLFVVSLSLLHLYS | |
| D2-D3 | TGVHSGKPIPNPLLGLDSTYIKPTLSAQPSPVVNSGGNVILQCDSQVAFDGF | 51 |
| domain | SLCKEGEDEHPQCLNSQPHARGSSRAIFSVGPVSPSRRWWYRCYAYDSNSPY | |
| | EWSLPSDLLELLVLGVSKKPSLSVQPGPIVAPEETLTLQCGSDAGYNRFVLY | |
| | KDGERDFLQLAGAQPQAGLSQANFTLGPVSRSYGGQYRCYGAHNLSSEWSAP | |
| | SDPLDILIAGQGALQSTASLFVVSLSLLHLYS | |
| D3-D4 | TGVHSGKPIPNPLLGLDSTSKKPSLSVQPGPIVAPEETLTLQCGSDAGYNRF | 52 |
| domain | VLYKDGERDFLQLAGAQPQAGLSQANFTLGPVSRSYGGQYRCYGAHNLSSEW | |
| | SAPSDPLDILIAGQFYDRVSLSVQPGPTVASGENVTLLCQSQGWMQTFLLTK | |
| | EGAADDPWRLRSTYQSQKYQAEFPMGPVTSAHAGTYRCYGSQSSKPYLLTHP | |
| | SDPLELVVSGPSGGPSSPTTGPTSTSGPEDQPLTPTGSDPQSGLGRHLGVVI | |
| | GILVAVILLLLLLLLLFLILRHRRQGKHWTSTQRKADFQHPAGAVGPEPTDR | |
| | GLQWRSSPAADAQEENLYAAVKHTQPEDGVEMDTRSPHDEDPQAVTYAEVKH | |
| | SRPRREMASPPSPLSGEFLDTKDRQAEEDRQMDTEAAASEAPQDVTYAQLHS | |
| | LTLRREATEPPPSQEGPSPAVPSIYATLAIH | |
| D1-D2-D3 | TGVHSGKPIPNPLLGLDSTGHLPKPTLWAEPGSVITQGSPVTLRCQGGQETQ | 53 |
| domain | EYRLYREKKTALWITRIPQELVKKGQFPIPSITWEHAGRYRCYYGSDTAGRS | |
| | ESSDPLELVVTGAYIKPTLSAQPSPVVNSGGNVILQCDSQVAFDGFSLCKEG | |
| | EDEHPQCLNSQPHARGSSRAIFSVGPVSPSRRWWYRCYAYDSNSPYEWSLPS | |
| | DLLELLVLGVSKKPSLSVQPGPIVAPEETLTLQCGSDAGYNRFVLYKDGERD | |
| | FLQLAGAQPQAGLSQANFTLGPVSRSYGGQYRCYGAHNLSSEWSAPSDPLDI | |
| | LIAGQGALQSTASLFVVSLSLLHLYS | |
| D2-D3-D4 | TGVHSGKPIPNPLLGLDSTYIKPTLSAQPSPVVNSGGNVILQCDSQVAFDGF | 54 |
| domain | SLCKEGEDEHPQCLNSQPHARGSSRAIFSVGPVSPSRRWWYRCYAYDSNSPY | |
| | EWSLPSDLLELLVLGVSKKPSLSVQPGPIVAPEETLTLQCGSDAGYNRFVLY | |
| | KDGERDFLQLAGAQPQAGLSQANFTLGPVSRSYGGQYRCYGAHNLSSEWSAP | |
| | SDPLDILIAGQFYDRVSLSVQPGPTVASGENVTLLCQSQGWMQTFLLTKEGA | |
| | ADDPWRLRSTYQSQKYQAEFPMGPVTSAHAGTYRCYGSQSSKPYLLTHPSDP | |
| | LELVVSGPSGGPSSPTTGPTSTSGPEDQPLTPTGSDPQSGLGRHLGVVIGIL | |
| | VAVILLLLLLLLLFLILRHRRQGKHWTSTQRKADFQHPAGAVGPEPTDRGLQ | |
| | WRSSPAADAQEENLYAAVKHTQPEDGVEMDTRSPHDEDPQAVTYAEVKHSRP | |
| | RREMASPPSPLSGEFLDTKDRQAEEDRQMDTEAAASEAPQDVTYAQLHSLTL | |
| | RREATEPPPSQEGPSPAVPSIYATLAIH | |

Results

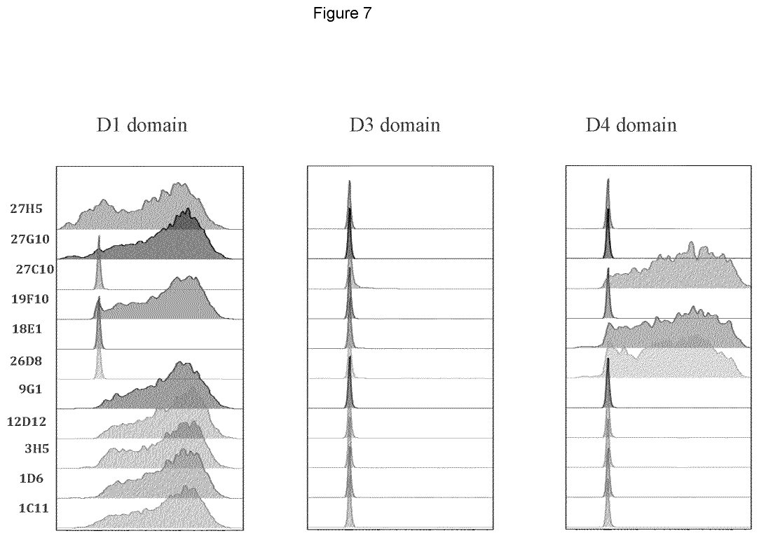

The ILT2 selective antibodies were tested for their binding to the different anchored ILT2 fragments by flow cytometry. 3H5, 12D12 and 27H5 all bound to the D1 domain of ILT2. These antibodies bound to all cells that expressed proteins that contained the D1 domain of ILT2, (the proteins of SEQ ID NOS: 46, 50 and 53) without binding to any of the cells that expressed the ILT2 proteins that lacked the D1 domain (the proteins of SEQ ID NOS: 47-49, 51, 52 and 54). The antibodies 3H5, 12D12 and 27H5 thus bind to a domain of ILT2 defined by residues 24-121 of the sequence shown in SEQ ID NO: 1 (also referred to as domain D1). Antibodies 26D8, 18E1 and 27C10 all bound to the D4 domain of ILT2. These antibodies bound to all cells that expressed proteins that contained the D4 domain of ILT2, (the proteins of SEQ ID NOS: 49, 52 and 54) without binding to any of the cells that expressed the ILT2 proteins that lacked the D4 domain (the proteins of SEQ ID NOS: 46-28, 50, 51, or 53). The antibodies 26D8, 18E1 and 27C10 thus bind to a domain of ILT2 defined by residues 322-458 of the sequence shown in SEQ ID NO: 1. FIG. 7 shows a representative example binding of the antibodies to the anchored ILT2 domain D1 fragment protein of SEQ ID NO: 46 (left hand panel), the D3 domain fragment protein of SEQ ID NO: 48 (middle panel), and the D4 domain protein of SEQ ID NO: 49 (right hand panel).

### ILT2 Point Mutation Study

The identification of antibodies that bound ILT2 without binding to the closely related ILT6 permitted the design of ILT2 mutations on amino acids exposed and different between ILT2 and ILT6. Anti-ILT2 antibodies that did not cross-react on ILT6 could then be mapped for loss of binding to different ILT2 mutants having amino acid substitutions in the D1, D2 or D4 domains of ILT2. The loss of binding to an ILT2 mutant together with loss of binding to human ILT6 can serve to identify to epitope on ILT2 bound by the antibodies that enhance NK cell cytotoxicity.

### Generation of ILT2 Mutants

ILT2 mutants were generated by PCR. The sequences amplified were run on agarose gel and purified using the Macherey Nagel PCR Clean-Up Gel Extraction kit (reference 740609). The purified PCR products generated for each mutant were then ligated into an expression vector, with the ClonTech InFusion system. The vectors containing the mutated sequences were prepared as Miniprep and sequenced. After sequencing, the vectors containing the mutated sequences were prepared as Midiprep using the Promega PureYield™ Plasmid Midiprep System. HEK293T cells were grown in DMEM medium (Invitrogen), transfected with vectors using Invitrogen's Lipofectamine 2000 and incubated at 37° C. in a CO2 incubator for 48 hours prior to testing for transgene expression. Mutants were transfected in Hek-293T cells, as shown in the table below. The targeted amino acid mutations are shown in the Table 6 below, listing the residue present in wild-type ILT2/position of residue/residue present in mutant ILT2, with position reference being to either the ILT2 protein lacking leader peptide shown in SEQ ID NO: 2 in the left column, or to the ILT2 protein with leader peptide shown in SEQ ID NO: 1 in the right column.

TABLE 6

| | Amino acid substitutions with | Amino acid substitutions with reference |
|---|---|---|
| | reference to ILT2 lacking leader | to ILT2 having leader peptide of SEQ ID |
| Mutant | peptide of SEQ ID NO: 2 | NO: 1 |
| 1 | G29S-Q30L-T32A-Q33A-D80H | G52S-Q53L-T55A-Q56A-D103H |
| 2 | E34A-R36A-Y76I-A82S-R84L | E57A-R59A-Y99I-A105S-R107L |
| 3 | Y99A-I100S-V126S-A127S- | Y122A-I123S-V149S-A150S- |
| | D129A-N180R-S181A-E184G | D152A-N203R-S204A-E207G |
| 3b | Q18A-W67A-Y99A-I100S-V126S- | Q41A-W90A-Y122A-I123S-V149S- |
| | S181A-E184G | S204A-E207G |
| 4 | S132A-L145S-N146A-Q148H- | S155A-L168S-N169A-Q171H- |
| | P149S | P172S |
| 5 | A127S-D129A-Q148H-R152A- | A150S-D152A-Q171H-R175A- |
| | N180R | N203R |
| 6 | Q107L-P108A-I119A-R156A | Q130L-P131A-I142A-R179A |
| 7 | P166A-R169A-W171S-L191A- | P189A-R192A-W194S-L214A- |
| | E193G-L195S-L197P | E216G-L218S-L220P |
| 8 | V111S-N113A-L195S-L197P | V134S-N136A-L218S-L220P |
| 4-1 | F299I-Y300R-D301A-W328G- | F322I-Y323R-D324A-W351G- |
| | Q378A-K381N | Q401A-K404N |
| 4-1b | Y300R-D301A-R302A-S304F- | Y323R-D324A-R325A-S327F- |
| | H387A-D390A | H410A-D413A |
| 4-2 | W328G-Q330H-R347A-T349A- | W351G-Q353H-R370A-T372A- |
| | Y350S-Y355A | Y373S-Y378A |
| 4-3 | Q324A-Q326S-S352A-Q353H- | Q347A-Q349S-S375A-Q376H- |
| | K354A | K377A |
| 4-4 | Q308A-P309G-N318A-T320A- | Q331A-P332G-N341A-T343A- |
| | E358S-G362S | E381S-G385S |
| 4-5 | D341A-D342S-W344L-R345A- | D364A-D365S-W367L-R368A- |
| | R347A | R370A |

### Results

The ILT2 selective antibodies were tested for their binding to each of mutants by flow cytometry. A first experiment was performed to determine antibodies that lose their binding to one or several mutants at one concentration. To confirm a loss of binding, titration of antibodies was done on antibodies for which binding seemed to be affected by the ILT2 mutations. A loss or decrease of binding for a test antibody indicated that one or more, or all of, the residues of the particular mutant are important to the core epitope of the antibodies, and thereby permitted the region of binding of ILT2 to be identified.

Antibodies 3H5, 12D12 and 27H5 bound an epitope in domain D1 of ILT2, as these three antibodies lost binding to mutant 2 having amino acid substitutions at residues 34, 36, 76, 82 and 84 (substitutions E34A, R36A, Y76I, A82S, R84L) in the domain 1 (D1 domain) of ILT2. 12D12 and 27H5 did not lose binding to any other mutant, however 3H5 also had a decrease (partial loss) of binding to mutant 1 having amino acid substitutions at residues 29, 30, 33, 32, 80 (substitutions G29S, Q30L, Q33A, T32A, D80H). These amino acid residues, together with lack of binding to human ILT6 polypeptide, therefore can identify an epitope that characterizes anti-ILT2 antibodies that enhance cytotoxicity in primary NK cells.

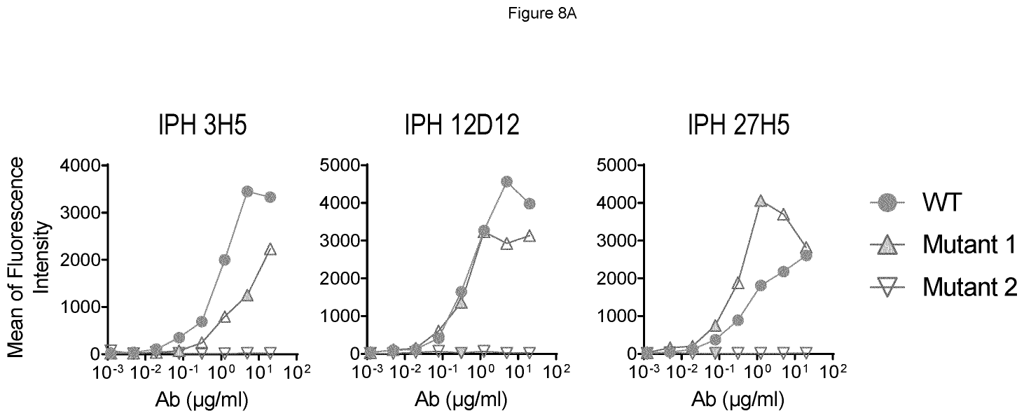

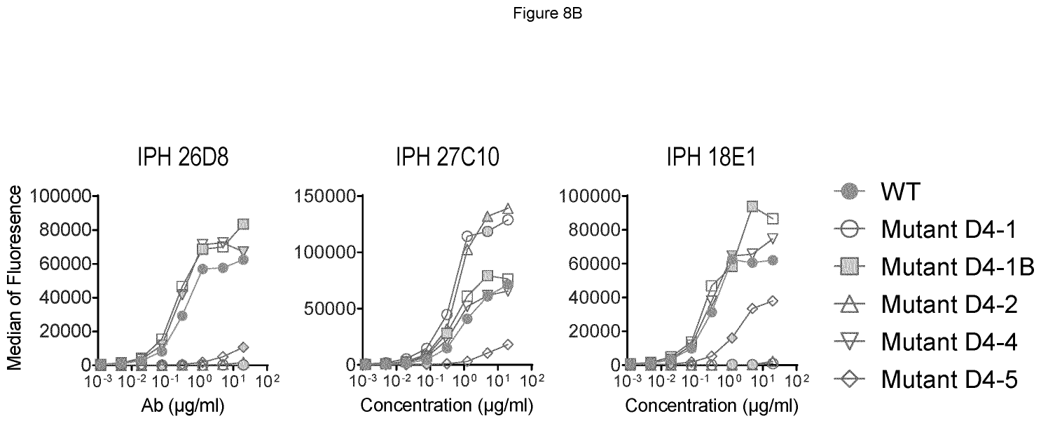

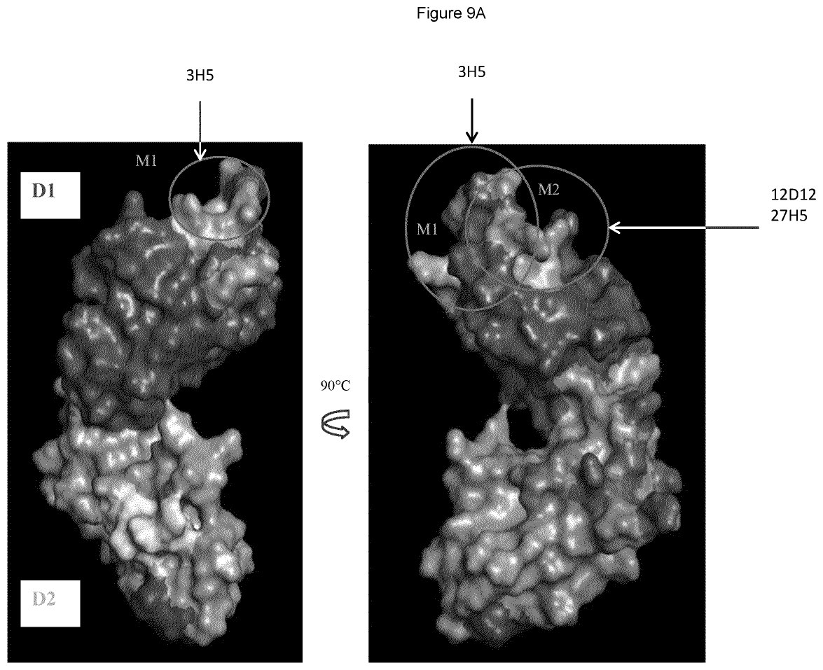

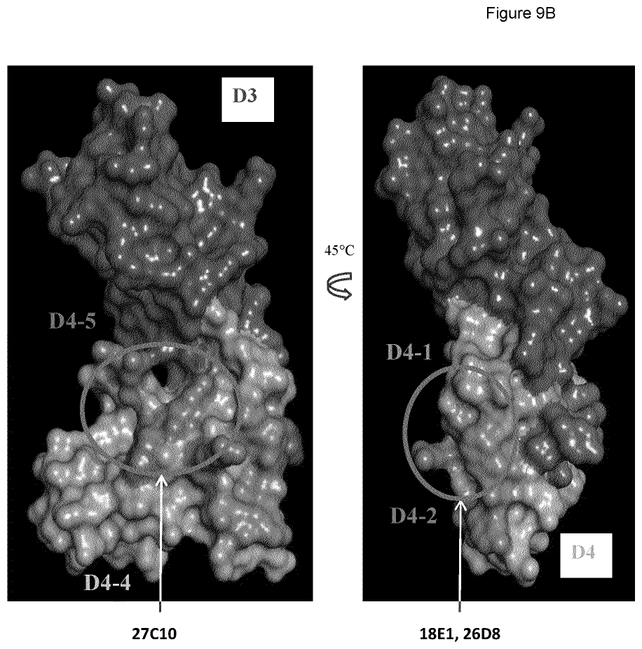

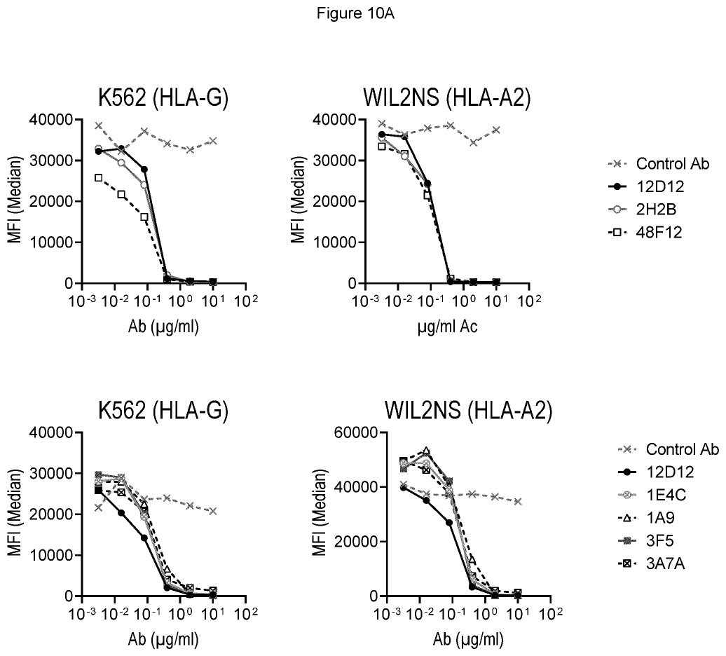

FIG. 8A shows a representative example of titration of antibodies 3H5, 12D12 and 27H5 for binding to mutants 1 and 2 by flow cytometry. FIG. 9A shows a model representing a portion of the ILT2 molecule that includes domain 1 (top portion, shaded in dark gray) and domain 2 (bottom, shaded in light gray). The figure shows the binding site of the antibodies as defined by the amino acid residues substituted in mutant 1 (M1) and mutant 2 (M2).

Antibodies 26D8, 18E1 and 27C10 all bound an epitope in domain D4 of ILT2. Antibodies 26D8 and 18E1 lost binding to mutants 4-1 and 4-2. Mutant 4-1 has amino acid substitutions at residues 299, 300, 301, 328, 378 and 381 (substitutions F299I, Y300R, D301A, W328G, Q378A, K381N). Mutant 4-2 has amino acid substitutions at residues 328, 330, 347, 349, 350 and 355 (substitutions W328G, Q330H, R347A, T349A, Y350S, Y355A). 26D8 furthermore lost binding to mutant 4-5, while antibody 18E1 had a decrease in binding (but not complete loss of binding) to mutant 4-5. 27C10 also lost binding to mutant 4-5, but not to any other mutant. Mutant 4-5 has amino acid substitutions at residues 341, 342, 344, 345 and 347 (substitutions D341A, D342S, W344L, R345A, R347A). 26D8 and 18E1 did not lose binding to any other mutants. These amino acid residues, together with lack of binding to human ILT6 polypeptide, therefore can identify an epitope that characterizes anti-ILT2 antibodies that enhance cytotoxicity in primary NK cells.

FIG. 8B shows a representative example of titration of antibodies 26D8, 18E1 and 27C10 for binding to D4 domain mutants 4-1, 4-1b, 4-2, 4-4 and 4-5 by flow cytometry

FIG. 9B shows a model representing a portion of the ILT2 molecule that includes domain 3 (top portion, shaded in dark gray) and domain 4 (bottom, shaded in light gray). The figure shows the binding site of the antibodies as defined by the amino acid residues substituted in mutants, 4-1, 4-2 and 4-5 which are all located within domain 4 of ILT2. Antibodies 26D8, 18E1 which potentiate the cytotoxicity of primary NK cells bind the site defined by mutants 4-1 and 4-2 without binding to the site defined by mutant 4-5, while antibodies 27C10 which did not potentiate the cytotoxicity of primary NK cells binds to the site defined by mutant 4-5.

### Example 12: Affinity Binding Threshold for Enhancement of Cytotoxicity in Primary Human NK Cells by ILT2-HLA-G Blocking Antibodies

In order to better understand the mechanism underlying the activity of the anti-ILT2 antibodies that were highly active in enhancing primary NK cell cytotoxicity, a further immunization and screening was carried out using the methods described in Example 3, combined with additional screening for binding to closely related ILT family members as in Example 10.

Balb/c mice were immunized with ILT-2_6×His protein. After the immunization protocol the mice were sacrificed to perform fusions and get hybridomas. The hybridoma supernatants were used to stain ILT-expressing CHO-cell lines described in Example 10 (CHO lines each expressing one of ILT1 (LILRA2), ILT3 (LILRB4), ILT4 (LILRB2), ILT5 (LILRB3), ILT6 (LILRA3) or ILT7 (LILRA4) to check for monoclonal antibody reactivities in a flow cytometry experiment. Briefly, the cells were incubated with 50 μl of supernatant for 1H at 4° C., washed three times and a secondary antibody Goat anti-mouse IgG Fc specific antibody coupled to AF647 was used (Jackson Immunoresearch, J1115-606-071). After 30 min of staining, the cells were washed three times and analyzed using a FACS CANTO II (Becton Dickinson).

Antibodies were cloned and screened, to identify those producing antibodies that bind to ILT2 without binding to human ILT1, ILT3, ILT4, ILT5, ILT6, or ILT7 and which have the ability to block the interaction between ILT2 with HLA-G. Briefly, recombinant biotinylated ILT2 was incubated with APC-conjugated streptavidin for 20 min at 4° C. prior addition of purified anti-ILT2 antibodies. After 20 min, the complexes were incubated with 5×10⁴ K562 cells expressing HLA-G or WIL2-NS cells expressing HLA-A2 for 30 supplemental min at 4° C. Cells were washed once in PBS and fixed with Cell Fix (Becton Dickinson, 340181). Analysis was performed on a FACS CANTO II flow cytometer.

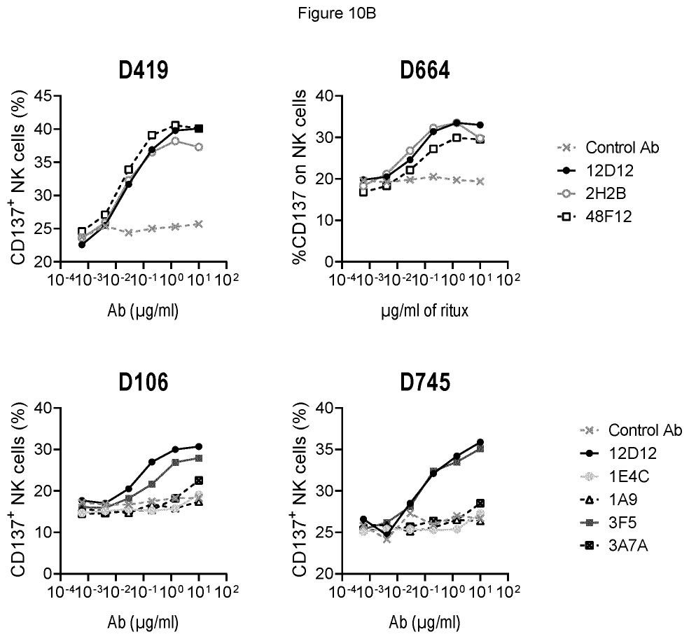

Ability of anti-ILT2 antibodies to block the interactions between HLA-G or HLA-A2 expressed at the surface of cell lines and recombinant ILT2 protein was assessed by flow cytometry, as described in Example 5. These assays permitted the identification of a panel of anti-ILT2 antibodies that were highly effective in blocking the interaction of ILT2 with its HLA class I ligand HLA-G. Antibodies 12D12, 2A8A, 2A8B, 2A9, 2B11, 2C4, 2C8, 2D8, 2E2B, 2E2C, 2E8, 2E11, 2G5, 2H2A, 2H2B, 2H12, 1A9, 1A10B, 1A10C, 1A10D, 1E4B, 1E4C, 3A7A, 3A7B, 3A8, 3B5, 3E5, 3E7A, 3E7B, 3E9A, 3E9B, 3F5, 4A8, 4C11B, 4E3A, 4E3B, 4H3, 5C5, 5D9, 6C6, 10H1, 48F12, 15D7, 2C3 all blocked ILT2 binding to HLA-G and HLA-A2. FIG. 10A shows representative results for antibodies 12D12, 2H2B, 48F12, 1E4C, 1A9, 3F5 and 3A7A. The resulting antibodies were tested for their binding to the different anchored ILT2 fragments and ILT2 point mutants by flow cytometry as shown in Example 11, and produced as modified chimeric antibodies having human IgG1 Fc domains with the mutations L234A/L235E/G237A/A330S/P331S.

Ability of anti-ILT2 antibodies to increase cytotoxicity in primary human NK cells was tested as in Example 9. Briefly, the effect of the anti-ILT2 antibodies on NK cells activation was determined by flow cytometry of CD137 expression on total NK cells, ILT2-positive NK cells and ILT2-negative NK cells. Effector cells were primary NK cells (fresh NK cells purified from donors, incubation overnight at 37° C. before use) and target cells (WIL2-NS cell line) were mixed at a ratio 1:1.

FIG. 10B is a representative figure showing the increase of % of total NK cells expressing CD137 mediated by anti-ILT2 antibodies 12D12, 2H2B, 48F12, 1E4C, 1A9, 3F5 and 3A7A using NK cells from two human donors and WIL2-NS that endogenously express HLA-A2. Antibodies showed strong activation of the primary NK cells. Study of ILT2-positive NK cells showed that these antibodies mediated a strong increase in activation of the NK cells toward the target cells. The characterization of their epitope on the point mutants showed that similarly to antibodies 3H5, 12D12 and 27H5, the antibodies 2H2B, 48F12 and 3F5 that were tested for domain binding all bound to the D1 domain of ILT2; they bound to all cells that expressed proteins that contained the D1 domain of ILT2, (the proteins of SEQ ID NOS: 46, 50 and 53) without binding to any of the cells that expressed the ILT2 proteins that lacked the D1 domain (the proteins of SEQ ID NOS: 47-49, 51, 52 and 54). When tested for binding to ILT-2 point mutants, Antibodies 12D12, 2H2B, 48F12, 1E4C, 1A9, 3F5 and 3A7A bound an epitope in domain D1 of ILT2, with loss of binding to mutant 2 having amino acid substitutions at residues 34, 36, 76, 82 and 84 (substitutions E34A, R36A, Y76I, A82S, R84L) in the domain 1 (D1 domain) of ILT2.

These results led to the observation that surprisingly some antibodies that were effective in blocking the interactions between HLA-G or HLA-A2 expressed at the surface of cell and bound the same area on the D1 domain of ILT2 were not necessarily able to mediate a an increase in or restore cytotoxicity of the primary human NK cells. In particular, as shown in FIG. 10B, antibodies 1E4C, 1A9 and 3A7A, despite being from the same murine V gene combinations as other antibodies (1E4C, 1A9 and 3A7A were from IGHV1-66*01 or IGHV1-84*01 genes combined with IGKV3-5*01), substantially lacked the ability to activate the primary NK cells all, compared to isotype control antibodies. Epitope mapping showed that these antibodies indeed bound to the D1 domain of ILT2; they bound to all cells that expressed proteins that contained the D1 domain of ILT2, (the proteins of SEQ ID NOS: 46, 50 and 53) without binding to any of the cells that expressed the ILT2 proteins that lacked the D1 domain (the proteins of SEQ ID NOS: 47-49, 51, 52 and 54), and that they showed loss of binding to mutant 2 having amino acid substitutions at residues 34, 36, 76, 82 and 84 (substitutions E34A, R36A, Y76I, A82S, R84L) in the domain 1 (D1 domain) of ILT2.

As part of an investigation into why these anti-D1 epitope antibodies did not function to enhance NK cell cytotoxicity in primary NK cells, we observed that for several antibodies that activated primary NK cells, there were also other antibodies having closely related variable region sequences which did not activate primary NK cells (despite being potent ILT2-HLA-G blockers. It may therefore be that the differences (in CDR residues in particular) may affect the affinity of the antibodies. The antibodies with CDRs derived from the same variable region genes were grouped and further characterized for their monovalent binding affinity to human ILT2 using the methods of Example 8. Briefly, anti-ILT2 antibodies at 1 μg/mL were captured onto a Protein-A chip and recombinant human ILT2 proteins were injected at 5 μg/mL over captured antibodies. For blank subtraction, cycles were performed again replacing ILT2 proteins with running buffer. The monovalent affinity analysis was conducted following a regular Capture-Kinetic protocol as recommended by the manufacturer (Biacore GE Healthcare kinetic wizard). Results are shown in Table 5, below. The antibodies 1E4C, 1A9 and 3A7A that blocked HLA-G and HLA-A2 but that did not enhance cytotoxicity of the primary human NK cells engaged the ILT-2 protein rapidly (ka in Table 5), however were characterized by a fast dissociation compared to the antibodies that are able to enhance cytotoxicity of the primary human NK cells. In particular, 1E4C, 1A9 and 3A7A were characterized by a 2 state reaction, in which the antibodies dissociate in two phases, a first rapid “kd1” phase and a second slower “kd2” phase. The first phase for 1E4C, 1A9 and 3A7A was characterized by a kd of greater than 1E-2. It therefore appears that while strong affinity in binding (on rate) may suffice to block the ILT2-HLA-G/A2 interaction in in vitro assays, a lower dissociation rate is required to enhance NK cell cytotoxicity. Differences in KD between the different D1 domain epitope antibodies was also generally observed, although less important than the kd. Results show that despite the ability of the anti-D1 domain epitope antibodies to potently block the interaction of ILT-2 with its HLA ligands, there is a threshold of affinity that is required to enhance NK cell cytotoxicity in primary NK cells.

Antibodies 2A8A, 2A9, 2C4, 2C8, 2D8, 2E2B, 2E2C, 2E8, 2E11, 2H2A, 2H12, 1A10D, 3E5, 3E7A, 3E7B, 3E9B, 3F5, 4C11B, 4E3A, 4E3B, 4H3, 5D9 and 6C6 had heavy chain variable region/CDRs derived from the murine IGHV1-66*01 gene and light chain variable region/CDRs derived from the murine IGKV3-5*01 gene. 1E4B had heavy chain variable region/CDRs derived from the murine IGHV1-66*01 gene and light chain variable region/CDRs derived from the murine IGKV3-4*01. 2H2B had heavy chain variable region/CDRs derived from the murine IGHV1-84*01 gene and light chain variable region/CDRs derived from the murine IGKV3-5*01 gene. The antibodies that activated primary NK cells displayed variable residues present at various positions in their VH and HCDRs as Kabat positions 32-35, 52A, 54, 55, 56, 57, 58, 60, 65, 95 and 101, and variable residues present at various positions in their VL and LCDRs as Kabat positions 24, 25, 26, 27, 27A, 28, 33, 34, 50, 53, 55, 91, 94 and 96.

48F12 had heavy chain variable region/CDRs derived from the murine IGHV2-3*01 gene and light chain variable region/CDRs derived from the murine IGKV10-96*02 gene.

The NK cell cytotoxicity-enhancing anti-D1 epitope antibodies 12D12, 2A8A, 2A9, 2C4, 2C8, 2D8, 2E2B, 2E2C, 2E8, 2E11, 2H2A, 2H2B, 2H12, 1A10D, 1E4B, 3E5, 3E7A, 3E7B, 3E9B, 3F5, 4C11B, 4E3A, 4E3B, 4H3, 5D9, 6C6 or 48F12 were characterized by a loss of binding to cells expressing ILT2 mutant 2 having amino acid substitutions at residues 34, 36, 76, 82 and 84 (substitutions E34A, R36A, Y76I, A82S, R84L), loss of binding to the human ILT-6 polypeptide, along with 1:1 Binding fit and/or dissociation or off rate (kd (1/s)) of less than 1E-2 or 1E-3 (monovalent binding affinity assay, as determined by SPR).

TABLE 5

| mAb | Fit | KD (nM) | ka (1/Ms) | kd (1/s) |
|---|---|---|---|---|
| 1A9 | Two State | 7.5 | ka1: 3.4E+5 | kd1: 3.5E−2 |
| | Reaction | | ka2: 2.7E−3 | kd2: 2.1E−4 |
| 1E4C | Two State | 1.8 | ka1: 1.1E+6 | kd1: 3.0E−2 |
| | Reaction | | ka2: 1.9E−3 | kd2: 1.3E−4 |
| 3A7A | Two State | 3.7 | ka1: 8.6E+5 | kd1: 3.1E−2 |
| | Reaction | | ka2: 1.8E−3 | kd2: 2.1E−4 |
| 2H2B | 1:1 Binding | 0.8 | 1.4E+6 | 1.1E−3 |
| 48F12 | 1:1 Binding | 0.2 | 5.0E+5 | 1.0E−4 |
| 3F5 | 1:1 Binding | 1.9 | 1.2E+6 | 2.2E−3 |

### Example 13: Antibodies Enhance NK Cell-Mediated ADDC

### Anti-ILT2 Antibodies Enhance NK Cell Cytotoxicity of Rituximab Towards Tumor Cells

The effect of the anti-ILT2 antibodies on NK cell activation was determined by analysis by flow cytometry of CD137 expression on NK cells, ILT2-positive NK cells and ILT2-negative NK cells from human tumor cells.

Tumor target cells were WIL2-NS tumor target cells in which ILT-2 was silenced. Effector cells (fresh NK cells purified from human healthy donors) and tumor target cells were mixed at a ratio 1:1. The CD137 assay was carried out in 96 U well plates in completed RPMI, 200 μL final/well. Antibodies used included anti-ILT-2 antibodies 12D12, 18E1 and 26D8 at a concentration of 10 μg/mL, isotype control antibodies, in combination with rituximab at a concentration of 0.001 μg/mL. Antibodies were pre-incubated 30 minutes at 37° C. with effector cells and then target cells were co-incubated overnight at 37° C. The following steps were: spin 3 min at 400 g; wash twice with Staining Buffer (SB); addition of 50 μL of staining Ab mix (anti-CD3 Pacific blue—BD Biosciences; anti-CD56-PE-Vio770—Miltenyi Biotec; anti-CD137-APC—Miltenyi Biotec; anti-ILT2-PE—clone HP-F1, eBioscience); incubation 30 min at 4° C.; wash twice with SB; resuspended pellet with Cellfix—Becton Dickinson; and fluorescence revealed with a FACS Canto II flow cytometer (Becton Dickinson). Negative controls were NK cells vs target cells alone and in presence of isotype control.

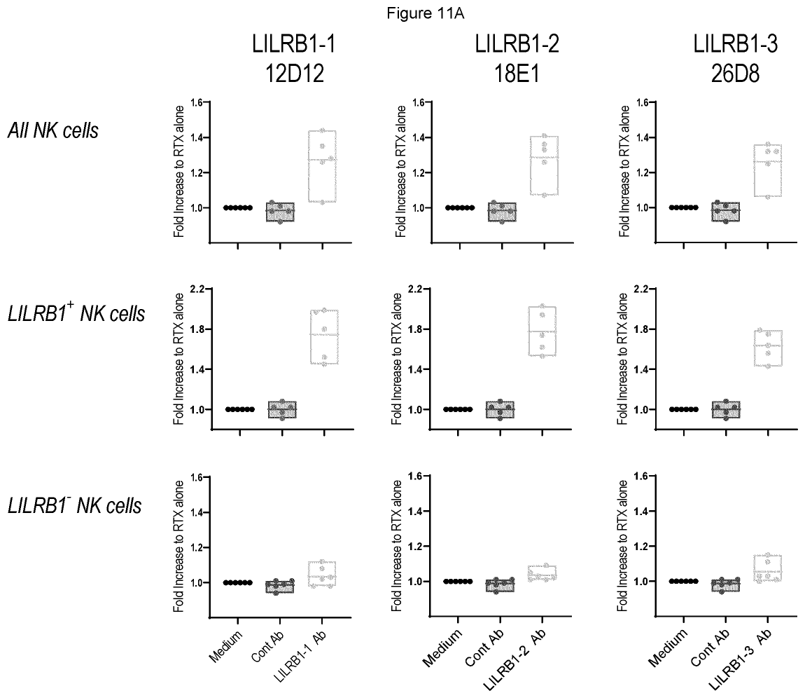

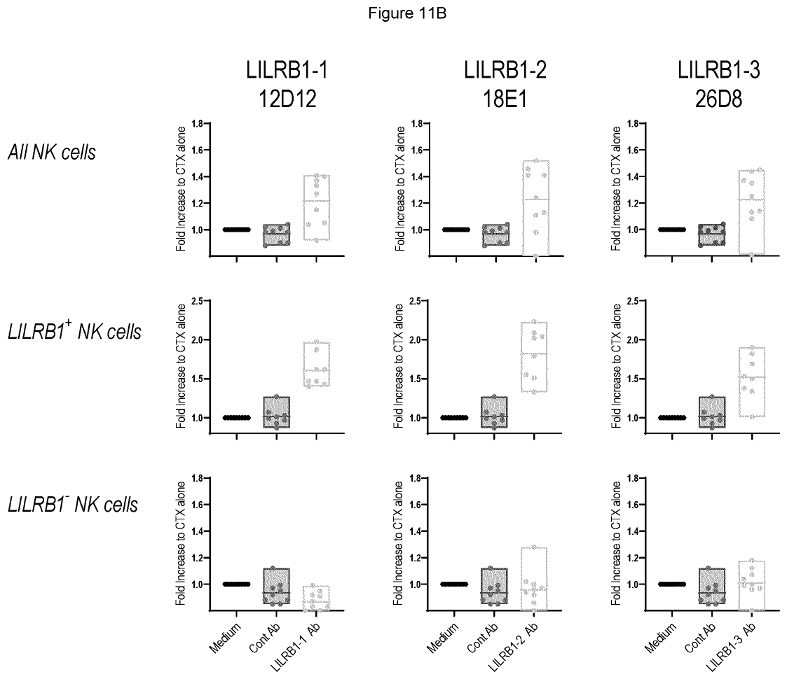

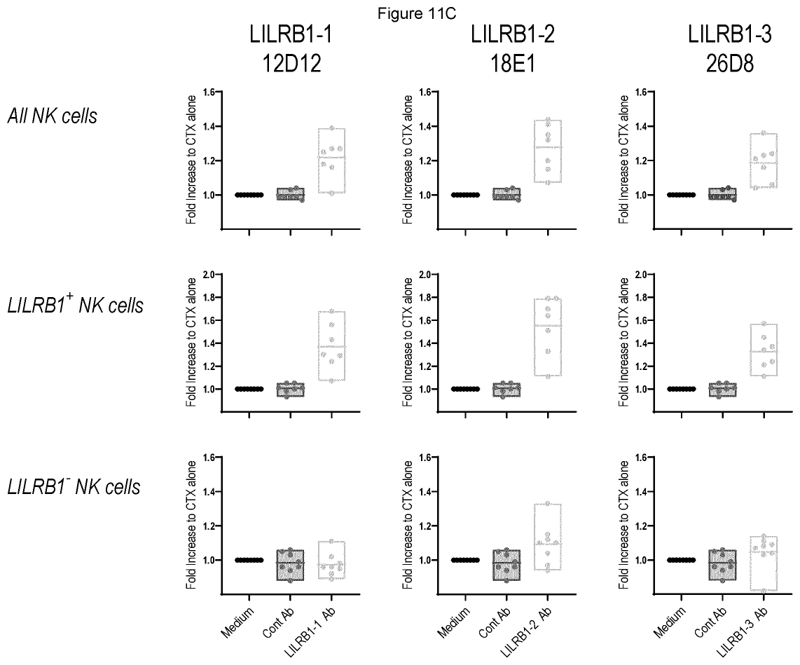

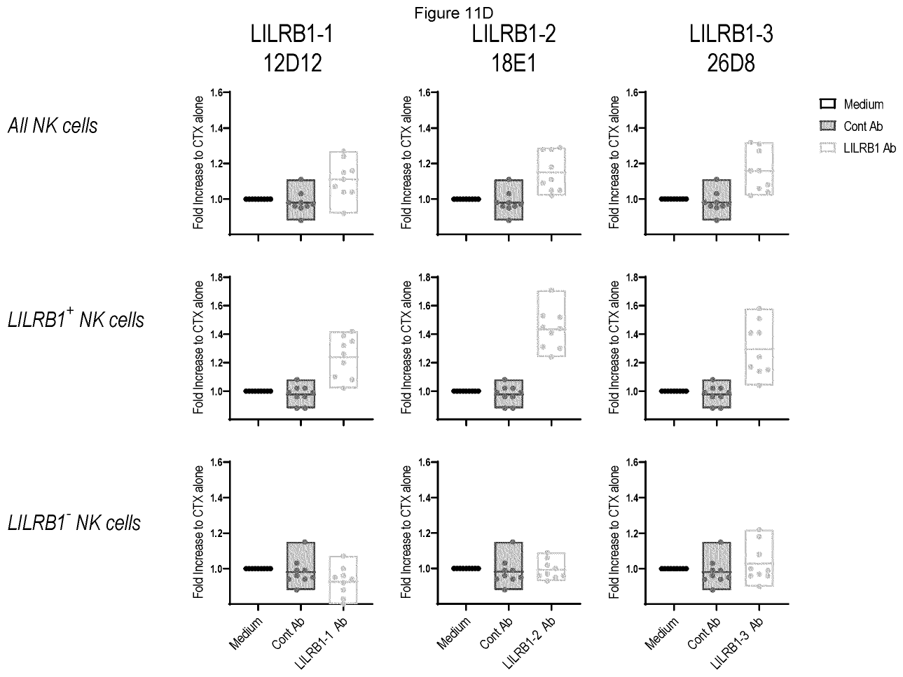

The anti-ILT2 antibodies were able to mediate a strong increase of NK cell cytotoxicity mediated by rituximab. Surprisingly, the combination of anti-ILT2 antibodies and rituximab resulted in stronger activation of total NK cell activation than either agent was able to mediate on its own. FIG. 11A shows the fold increase over rituximab alone (compared to medium) in activation of NK cells following incubation with rituximab without or without anti-ILT2 antibodies, and the tumor target cells, in five human donors. Each of the anti-ILT2 antibodies 12D12, 18E1 and 26D8 resulted in an increase of the NK cytotoxicity mediated by rituximab alone. The combination increased NK cell cytotoxicity of rituximab in the LILRB1+ population of NK cells and in the entire NK cell population.

### Anti-ILT2 Antibodies Enhance NK Cell Cytotoxicity of Cetuximab Towards Tumor Cells

The effect of the anti-ILT2 antibodies on NK cell activation was determined by analysis by flow cytometry of CD137 expression on NK cells, ILT2-positive NK cells and ILT2-negative NK cells from human tumor cells.

Tumor target cells were HN (human oral squamous cell carcinoma, DMSZ® ACC 417, FaDu (human pharynx tissue, HNSCC, ATCC® HTB-43) or Cal27 (human tongue tissue, HNSCC, ATCC® CRL-2095™). Effector cells (fresh NK cells purified from human healthy donors) and tumor target cells were mixed at a ratio 1:1. The CD137 assay was carried out in 96 U well plates in completed RPMI, 200 μL final/well. Antibodies used included anti-ILT-2 antibodies 12D12, 18E1 and 26D8 at a concentration of 10 μg/mL, isotype control antibodies, in combination with cetuximab at a concentration of 0.01 μg/mL. Antibodies were pre-incubated 30 minutes at 37° C. with effector cells and then target cells were co-incubated overnight at 37° C. The following steps were: spin 3 min at 400 g; wash twice with Staining Buffer (SB); addition of 50 μL of staining Ab mix (anti-CD3 Pacific blue—BD Biosciences; anti-CD56-PE-Vio770—Miltenyi Biotec; anti-CD137-APC—Miltenyi Biotec; anti-ILT2-PE—clone HP-F1, eBioscience); incubation 30 min at 4° C.; wash twice with SB; resuspended pellet with Cellfix—Becton Dickinson; and fluorescence revealed with a FACS Canto II flow cytometer (Becton Dickinson). Negative controls were NK cells vs target cells alone and in presence of isotype control.

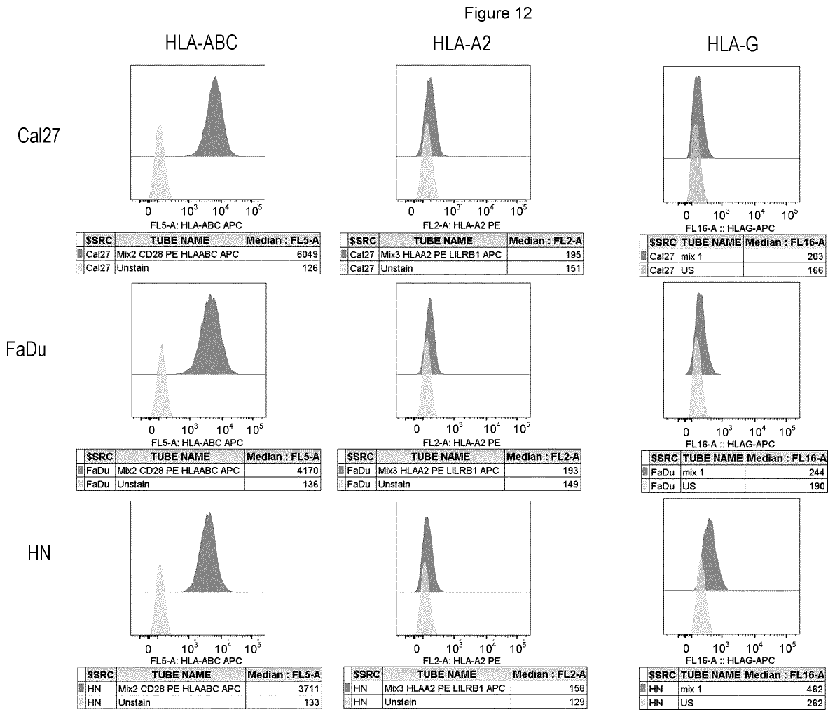

HNSCC tumor cells were found to be consistently negative for HLA-G and HLA-A2, as determined by flow cytometry, as shown in FIG. 12. However, despite the absence of the main known ligands of ILT2, the anti-ILT2 antibodies were able to mediate a strong increase of NK cell cytotoxicity mediated by cetuximab. Surprisingly, the combination of anti-ILT2 antibodies and cetuximab resulted in much stronger activation of total NK cell activation that either agent was able to mediate on its own. FIG. 11B shows the fold increase over cetuximab alone (compared to medium) in activation of NK cells following incubation with cetuximab with or without anti-ILT2 antibodies, and HN tumor target cells, in three human donors. FIG. 11C shows the fold increase over cetuximab alone (compared to medium) in activation of NK cells following incubation with cetuximab with or without anti-ILT2 antibodies, and FaDu tumor target cells, in three human donors. FIG. 11D shows the fold increase over cetuximab alone (compared to medium) in activation of NK cells following incubation with cetuximab with or without anti-ILT2 antibodies, and Cal27 tumor target cells, in three human donors. Each of the anti-ILT2 antibodies 12D12, 18E1 and 26D8 resulted in an increase of the NK cytotoxicity mediated by cetuximab alone. The combination increased NK cell cytotoxicity of cetuximab in the LILRB1+ population of NK cells and in the entire NK cell population.

### Example 14: Enhancement of Macrophage-Mediated ADCP

Antibodies were tested for the ability to enhance antibody-dependent cellular phagocytosis.

Briefly, monocyte derived macrophages from healthy donors were obtained after 6 to 7 days of culture in complete RPMI supplemented with 100 ng/mL of M-CSF in flat bottom 96 well plate (40000 cells/well). Antibody-dependent cell phagocytosis (ADCP) experiments were performed in RPMI without phenol red to avoid interference with the dye used to label target cells. Macrophages were starved in RPMI without FBS for 2 hours before addition of antibodies and target cells. A dose range of rituximab (10-1-0.1 μg/mL) and a fixed-dose of anti-ILT2 or control antibodies (10 μg/mL) were added on macrophages. 30000 cells/well HLA-A2-expressing target cells were labelled using ph-Rodo Red reagent (which is fluorescence at acidic pH in endocytic vesicles upon phagocytosis by macrophages), added to macrophages and incubated for 3 to 6 hours in the Incucyte-S3 imager. Images were acquired every 30 min. ADCP was quantified using total red objet integrated intensity (RCU×μm²/image) metrics.

Commercial anti-ILT2 antibody GHI/75 (mouse IgG2b isotype) and a variant (“HUB3”) thereof having human IgG1 Fc domains modified by introduction of the L234A/L235E/G237A/A330S/P331S mutations to substantially eliminate human FcγR binding were then tested for ability to increase rituximab-mediated phagocytosis by macrophages of HLA-A2-expressing B cells, compared to rituximab alone.

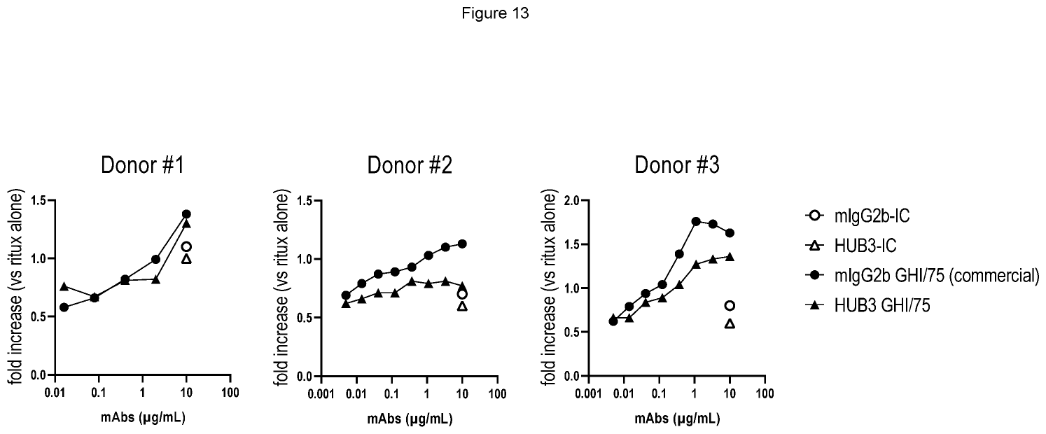

Results are shown in FIG. 13. The ILT2-blocking antibodies GHI/75 (commercial antibody, mouse IgG2b isotype) enhanced ADCP mediated by the anti-CD20 antibody rituximab in macrophages towards HLA-A2-expressing B cells (B104 cells). In comparison, the human IgG1 Fc-modified GHI/75 variant (HUB3 in FIG. 12) comprising the L234A/L235E/G237A/A330S/P331S mutations showed a decreased ability to enhance ADCP mediated by rituximab

The interactions between the Fc domain of anti-ILT2 antibodies and FcγR may therefore play an important role in the observed macrophage mediated cell death. This opens the possibility to modulate the ability of the anti-ILT2 antibodies to mediate ADCP through maintenance or inclusion of Fc domains that bind FcγR (e.g. native IgG1 domains) in order to mediate ADCP.

TABLE 6

| | | | | L234A/ | | | |
|---|---|---|---|---|---|---|---|
| | | | | L235E/ | L234A/ | Wild type | |
| | | L234F/ | L234A/ | G237A/ | L235E/ | human | Human IgG4 |
| | | L235E/ | L235E/ | A330S/ | G237A/ | IgG1 | antibody |
| Human Fc | N297S | P331S | P331S | P331S | P331S | antibody | with S241P |
| receptor | KD (nM) | KD (nM) | KD (nM) | KD (nM) | KD (nM) | KD (nM) | KD (nM) |
| CD64 | 278 | 933 | 1553 | No binding | No binding | 12.74 | 96.83 |
| CD32a | No binding | 14250 | 19900 | 18190 | 16790 | 2075 | 3218 |
| CD32b | No binding | 17410 | 79830 | 21800 | 16570 | 3914 | 2659 |
| CD16a(F) | No binding | 35580 | No binding | No binding | No binding | 961.9 | Low binding |
| CD16a(V) | No binding | 8627 | 9924 | No binding | No binding | 733.7 | 8511 |
| CD16b | No binding | No binding | No binding | No binding | No binding | 15020 | Low binding |
| FcRn | 712 | 627 | 1511 | 714 | 758 | 1272 | 1176 |

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law), regardless of any separately provided incorporation of particular documents made elsewhere herein.

Unless otherwise stated, all exact values provided herein are representative of corresponding approximate values (e.g., all exact exemplary values provided with respect to a particular factor or measurement can be considered to also provide a corresponding approximate measurement, modified by “about,” where appropriate). Where “about” is used in connection with a number, this can be specified as including values corresponding to +/−10% of the specified number.

The description herein of any aspect or embodiment of the invention using terms such as “comprising”, “having,” “including,” or “containing” with reference to an element or elements is intended to provide support for a similar aspect or embodiment of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

1-30. (canceled)

31. A method of treating an individual who has a head and neck squamous cell carcinoma (HNSCC), the method comprising administering to the individual an antibody that binds a human ILT-2 polypeptide and neutralizes the inhibitory activity of ILT-2 in combination with cetuximab.

32. The method of claim 31, wherein the antibody that binds a human ILT-2 polypeptide lacks an Fc domain or has a human Fc domain that is modified to reduce binding between the Fc domain and an Fcγ receptor.

33. The method of claim 31, wherein the antibody that binds a human ILT-2 polypeptide does not inhibit the binding of a soluble human ILT-6 protein to a HLA class I molecule.

34. The method of claim 31, wherein the individual has a HLA-G and/or HLA-A2 negative cancer.

35. The method of claim 34, wherein the treatment does not require a prior step of determining whether the individual has a HLA-G and/or HLA-A2 positive cancer.

36. The method of claim 31, wherein the antibody that binds a human ILT-2 polypeptide competes for binding to an ILT2 polypeptide of SEQ ID NO: 1 with an antibody comprising the heavy and light chain CDRs, or the heavy and light chain variable regions, of antibody 12D12, 3H5, 27H5, 26D8, 27C10 or 18E1.

37. The method of claim 31, wherein the antibody that binds a human ILT-2 polypeptide comprises a modified human IgG1 Fc domain comprising N-linked glycosylation at Kabat residue N297 and comprising an amino acid substitution at Kabat residue(s) 234 and 235, optionally further at Kabat residue 331, optionally at Kabat residues 234, 235, 237 and at Kabat residues 330 and/or 331, optionally wherein the Fc domain comprises L234A/L235E/P331S substitutions, L234F/L235E/P331S substitutions, L234A/L235E/G237A/P331S substitutions, or L234A/L235E/G237A/A330S/P331S substitutions.

38. The method of claim 31, wherein said antibody that binds ILT-2 is capable of enhancing the cytotoxicity of NK cells in a 4-hour in vitro ⁵¹Cr release cytotoxicity assay in which NK cells that express ILT2 are purified from human donors and incubated with target cells that express at their surface HLA-G polypeptides.

39. The method of claim 31, wherein the antibody that binds a human ILT-2 polypeptide does not inhibit the binding of a soluble human ILT-6 protein to a HLA class I molecule.

40. The method of claim 31, wherein the antibody has reduced binding to (i) a mutant ILT2 polypeptide comprising the mutations E34A, R36A, Y76I, A82S, R84L (with reference to SEQ ID NO: 2), (ii) a mutant ILT2 polypeptide comprising the mutations G29S, Q30L, Q33A, T32A, D80H (with reference to SEQ ID NO: 2), (iii) a mutant ILT2 polypeptide comprising the mutations F299I, Y300R, D301A, W328G, Q378A, K381N (with reference to SEQ ID NO: 2), or (iv) a mutant ILT2 polypeptide comprising the mutations W328G, Q330H, R347A, T349A, Y350S, Y355A (with reference to SEQ ID NO: 2), in each case relative to binding between the antibody and a wild-type ILT2 polypeptide comprising the amino acid sequence of SEQ ID NO: 2.

41. The method of claim 31, wherein the antibody that binds ILT-2 comprises the heavy and light chain CDR1, 2 and 3 of antibody 12D12, 26D8, 18E1, 2A8A, 2A9, 2C4, 2C8, 2D8, 2E2B, 2E2C, 2E8, 2E11, 2H2A, 2H12, 1A10D, 1E4B, 3E5, 3E7A, 3E7B, 3E9B, 4C11B, 4E3A, 4E3B, 4H3, 5D9, 6C6, 2H2B, 48F12, 3F5, 12D12, 3H5, 27H5, 26D8, 27C10 or 18E1.

42. The method of claim 31, wherein the antibody that binds ILT-2 and the antibody that binds EGFR are formulated for separate administration and are administered concurrently or sequentially.

43. A method of treating an individual who has an HNSCC, the method comprising administering to the individual an antibody that binds a human ILT-2 polypeptide and neutralizes the inhibitory activity of ILT-2 in combination with cetuximab, wherein the treatment is further in combination with an antibody that neutralizes the inhibitory activity of PD-1.

44. A pharmaceutical composition comprising an antibody that binds ILT-2 and an antibody that binds EGFR, wherein the antibody that binds ILT-2 has an Fc domain that is modified to reduce binding between the Fc domain and an Fcγ receptor.

45. A kit comprising a pharmaceutical composition containing an antibody that binds a human ILT-2 polypeptide and neutralizes the inhibitory activity of ILT-2, and a second pharmaceutical composition containing an anti-EGFR antibody, and instructions to administer said anti-EGFR antibody with an anti-ILT-2 antibody.


SEQUENCE LISTING (215 sequences)

SEQ ID NO: 1 - protein, 650 aa, homo sapiens
MTPILTVLICLGLSLGPRTHVQAGHLPKPTLWAEPGSVITQGSPVTLRCQGGQETQEYRLYREKKTALWITRIPQELVKKGQFPIPSITWEHAGRYRCYYGSDTAGRSESSDPLELVVTGAYIKPTLSAQPSPVVNSGGNVILQCDSQVAFDGFSLCKEGEDEHPQCLNSQPHARGSSRAIFSVGPVSPSRRWWYRCYAYDSNSPYEWSLPSDLLELLVLGVSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQFYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 2 - protein, 627 aa, homo sapiens
GHLPKPTLWAEPGSVITQGSPVTLRCQGGQETQEYRLYREKKTALWITRIPQELVKKGQFPIPSITWEHAGRYRCYYGSDTAGRSESSDPLELVVTGAYIKPTLSAQPSPVVNSGGNVILQCDSQVAFDGFSLCKEGEDEHPQCLNSQPHARGSSRAIFSVGPVSPSRRWWYRCYAYDSNSPYEWSLPSDLLELLVLGVSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQFYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 3 - protein, 435 aa, Homo sapiens
GHLPKPTLWAEPGSVIIQGSPVTLRCQGSLQAEEYHLYRENKSASWVRRIQEPGKNGQFPIPSITWEHAGRYHCQYYSHNHSSEYSDPLELVVTGAYSKPTLSALPSPVVTLGGNVTLQCVSQVAFDGFILCKEGEDEHPQRLNSHSHARGWSWAIFSVGPVSPSRRWSYRCYAYDSNSPYVWSLPSDLLELLVPGVSKKPSLSVQPGPMVAPGESLTLQCVSDVGYDRFVLYKEGERDFLQRPGWQPQAGLSQANFTLGPVSPSHGGQYRCYSAHNLSSEWSAPSDPLDILITGQFYDRPSLSVQPVPTVAPGKNVTLLCQSRGQFHTFLLTKEGAGHPPLHLRSEHQAQQNQAEFRMGPVTSAHVGTYRCYSSLSSNPYLLSLPSDPLELVVSASLGQHPQDYTVENLIRMGVAGLVLVVLGILLFEAQHSQR

SEQ ID NO: 4 - protein, 419 aa, homo sapiens
GPLPKPTLWAEPGSVISWGNSVTIWCQGTLEAREYRLDKEESPAPWDRQNPLEPKNKARFSIPSMTEDYAGRYRCYYRSPVGWSQPSDPLELVMTGAYSKPTLSALPSPLVTSGKSVTLLCQSRSPMDTFLLIKERAAHPLLHLRSEHGAQQHQAEFPMSPVTSVHGGTYRCFSSHGFSHYLLSHPSDPLELIVSGSLEGPRPSPTRSVSTAGPEDQPLMPTGSVPHSGLRRHWEVLIGVLVVSILLLSLLLFLLLQHWRQGKHRTLAQRQADFQRPPGAAEPEPKDGGLQRRSSPAADVQGENFCAAVKNTQPEDGVEMDTRQSPHDEDPQAVTYAKVKHSRPRREMASPPSPLSGEFLDTKDRQAEEDRQMDTEAAASEAPQDVTYAQLHSFTLRQKATEPPPSQEGASPAEPSVYA

SEQ ID NO: 5 - protein, 574 aa, homo sapiens
GTIPKPTLWAEPDSVITQGSPVTLSCQGSLEAQEYRLYREKKSASWITRIRPELVKNGQFHIPSITWEHTGRYGCQYYSRARWSELSDPLVLVMTGAYPKPTLSAQPSPVVTSGGRVTLQCESQVAFGGFILCKEGEEEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGPSMGSSPPPTGPISTPGPEDQPLTPTGSDPQSGLGRHLGVVIGILVAVVLLLLLLLLLFLILRHRRQGKHWTSTQRKADFQHPAGAVGPEPTDRGLQWRSSPAADAQEENLYAAVKDTQPEDGVEMDTRAAASEAPQDVTYAQLHSLTLRRKATEPPPSQEREPPAEPSIYATLAIH

SEQ ID NO: 6 - protein, 603 aa, homo sapiens
GPFPKPTLWAEPGSVISWGSPVTIWCQGSLEAQEYRLDKEGSPEPLDRNNPLEPKNKARFSIPSMTEHHAGRYRCHYYSSAGWSEPSDPLELVMTGFYNKPTLSALPSPVVASGGNMTLRCGSQKGYHHFVLMKEGEHQLPRTLDSQQLHSGGFQALFPVGPVNPSHRWRFTCYYYYMNTPQVWSHPSDPLEILPSGVSRKPSLLTLQGPVLAPGQSLTLQCGSDVGYDRFVLYKEGERDFLQRPGQQPQAGLSQANFTLGPVSPSHGGQYRCYGAHNLSSEWSAPSDPLNILMAGQIYDTVSLSAQPGPTVASGENVTLLCQSWWQFDTFLLTKEGAAHPPLRLRSMYGAHKYQAEFPMSPVTSAHAGTYRCYGSYSSNPHLLSHPSEPLELVVSGHSGGSSLPPTGPPSTPGLGRYLEVLIGVSVAFVLLLFLLLFLLLRRQRHSKHRTSDQRKTDFQRPAGAAETEPKDRGLLRRSSPAADVQEENLYAAVKDTQSEDRVELDSQSPHDEDPQAVTYAPVKHSSPRREMASPPSSLSGEFLDTKDRQVEEDRQMDTEAAASEASQDVTYAQLHSLTLRRKATEPPPSQEGEPPAEPSIYA

SEQ ID NO: 7 - protein, 411 aa, homo sapiens
GPLPKPTLWAEPGSVITQGSPVTLRCQGSLETQEYHLYREKKTALWITRIPQELVKKGQFPILSITWEHAGRYCCIYGSHTAGLSESSDPLELVVTGAYSKPTLSALPSPVVTSGGNVTIQCDSQVAFDGFILCKEGEDEHPQCLNSHSHARGSSRAIFSVGPVSPSRRWSYRCYGYDSRAPYVWSLPSDLLGLLVPGVSKKPSLSVQPGPVVAPGEKLTFQCGSDAGYDRFVLYKEWGRDFLQRPGRQPQAGLSQANFTLGPVSRSYGGQYTCSGAYNLSSEWSAPSDPLDILITGQIRARPFLSVRPGPTVASGENVTLLCQSQGGMHTFLLTKEGAADSPLRLKSKRQSHKYQAEFPMSPVTSAHAGTYRCYGSLSSNPYLLTHPSDPLELVVSGAAETLSPPQNKSD

SEQ ID NO: 8 - protein, 471 aa, homo sapiens
ENLPKPILWAEPGPVITWHNPVTIWCQGTLEAQGYRLDKEGNSMSRHILKTLESENKVKLSIPSMMWEHAGRYHCYYQSPAGWSEPSDPLELVVTAYSRPTLSALPSPVVTSGVNVTLRCASRLGLGRFTLIEEGDHRLSWTLNSHQHNHGKFQALFPMGPLTFSNRGTFRCYGYENNTPYVWSEPSDPLQLLVSGVSRKPSLLTLQGPVVTPGENLTLQCGSDVGYIRYTLYKEGADGLPQRPGRQPQAGLSQANFTLSPVSRSYGGQYRCYGAHNVSSEWSAPSDPLDILIAGQISDRPSLSVQPGPTVTSGEKVTLLCQSWDPMFTFLLTKEGAAHPPLRLRSMYGAHKYQAEFPMSPVTSAHAGTYRCYGSRSSNPYLLSHPSEPLELVVSGATETLNPAQKKSDSKTAPHLQDYTVENLIRMGVAGLVLLFLGILLFEAQHSQRSPPRCSQEANSRKDNAPFRVVE

SEQ ID NO: 9 - protein, 437 aa, homo sapiens
GPFPKPTLWAEPGSVISWGSPVTIWCQGSLEAQEYQLDKEGSPEPLDRNNPLEPKNKARFSIPSMTQHHAGRYRCHYYSSAGWSEPSDPLELVMTGFYNKPTLSALPSPVVASGGNMTLRCGSQKGYHHFVLMKEGEHQLPRTLDSQQLHSGGFQALFPVGPVTPSHRWRFTCYYYYTNTPRVWSHPSDPLEILPSGVSRKPSLLTLQGPVLAPGQSLTLQCGSDVGYDRFVLYKEGERDFLQRPGQQPQAGLSQANFTLGPVSPSHGGQYRCYGAHNLSSEWSAPSDPLNILMAGQIYDTVSLSAQPGPTVASGENVTLLCQSRGYFDTFLLTKEGAAHPPLRLRSMYGAHKYQAEFPMSPVTSAHAGTYRCYGSYSSNPHLLSFPSEPLELMVSASHAKDYTVENLIRMGMAGLVLVFLGILLFEAQHSQRNPQD

SEQ ID NO: 10 - protein, 338 aa, homo sapiens
MVVMAPRTLFLLLSGALTLTETWAGSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQSSLPTIPIMGIVAGLVVLAAVVTGAAVAAVLWRKKSSD

SEQ ID NO: 11 - protein, 358 aa, homo sapiens
MVDGTLLLLLSEALALTQTWAGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 12 - protein, 117 aa, mus musculus
QVQLQQSGAELVKPGASVKLSCKASGYTFTEHTIHWIKQRSGQGLEWIGWFYPGSGSMKYNEKFKDKATLTADKSSSTVYMELTRLTSEDSAVYFCARHTNWDFDYWGQGTTLTVSS

SEQ ID NO: 13 - protein, 111 aa, mus musculus
DIVLTQSPASLAVSLGQRATISCKASQSVDYGGDSYMNWYQQKPGQPPKLLIYAASNLESGIPARFSGSGSGTDLTLNIHPVEEDDAAMYYCQQSNEEPWTFGGGTKLEIK

SEQ ID NO: 14 - protein, 5 aa, mus musculus
EHTIH

SEQ ID NO: 15 - protein, 17 aa, mus musculus
WFYPGSGSMKYNEKFKD

SEQ ID NO: 16 - protein, 8 aa, Mus musculus
HTNWDFDY

SEQ ID NO: 17 - protein, 15 aa, Mus musculus
KASQSVDYGGDSYMN

SEQ ID NO: 18 - protein, 7 aa, Mus musculus
AASNLES

SEQ ID NO: 19 - protein, 9 aa, Mus musculus
QQSNEEPWT

SEQ ID NO: 20 - protein, 117 aa, Mus musculus
QVQLQQSGAELVKPGASVRLSCKASGYTFTAHTIHWVKQRSGQGLEWIGWLYPGSGSIKYNEKFKDKATLTADKSSSTVYMELSRLTSEDSAVYFCARHTNWDFDYWGQGTTLTVSS

SEQ ID NO: 21 - protein, 111 aa, Mus musculus
NIVLTQSPASLAVSLGQRATISCKASQSVDYGGASYMNWYQQKPGQPPKLLIYAASNLESGIPARFSGSGSGTDLTLNIHPVEEEDAAMYYCQQSNEEPWTFGGGTKLEIK

SEQ ID NO: 22 - protein, 5 aa, Mus musculus
AHTIH

SEQ ID NO: 23 - protein, 17 aa, Mus musculus
WLYPGSGSIKYNEKFKD

SEQ ID NO: 24 - protein, 8 aa, Mus musculus
HTNWDFDY

SEQ ID NO: 25 - protein, 15 aa, Mus musculus
KASQSVDYGGASYMN

SEQ ID NO: 26 - protein, 7 aa, Mus musculus
AASNLES

SEQ ID NO: 27 - protein, 9 aa, Mus musculus
QQSNEEPWT

SEQ ID NO: 28 - protein, 122 aa, Mus musculus
QVQLQQPGAELVKPGASVRMSCKASGYTFTSYWVHWVKQRPGQGLEWIGVIDPSDSYTSYNQNFKGKATLTVDTSSKTAYIHLSSLTSEDSAVYFCARGERYDGDYFAMDYWGQGTSVTVSS

SEQ ID NO: 29 - protein, 107 aa, Mus musculus
DIVMTQSPASLSVSVGETVTITCRASENIYSNLAWYQQKQGKSPQLLVYAATNLADGVPSRFSGSRSGTQYSLKINSLQSEDFGTYYCQHFWNTPRTFGGGTKLEIK

SEQ ID NO: 30 - protein, 5 aa, Mus musculus
SYWVH

SEQ ID NO: 31 - protein, 17 aa, Mus musculus
VIDPSDSYTSYNQNFKG

SEQ ID NO: 32 - protein, 13 aa, Mus musculus
GERYDGDYFAMDY

SEQ ID NO: 33 - protein, 11 aa, Mus musculus
RASENIYSNLA

SEQ ID NO: 34 - protein, 7 aa, Mus musculus
AATNLAD

SEQ ID NO: 35 - protein, 9 aa, Mus musculus
QHFWNTPRT

SEQ ID NO: 36 - protein, 118 aa, Mus musculus
QVQLKESGPGLVAPSQSLSITCTVSGFSLTSYGVSWVRQPPGKGLEWLGVIWGDGSTNYHSALISRLSISKDNSKSQVFLKLNSLQTDDTATYYCAKPRWDDYAMDYWGQGTSVTVSS

SEQ ID NO: 37 - protein, 106 aa, Mus musculus
DIQMTQTTSSLSASLGDRVTISCRASQDISNYLNWYQQKPDGTVKLLIYYTSRLHSGVPSRFSGSGSGTDYSLTISNLEQEDIATYFCQQGNTLWTFGGGTKLEIK

SEQ ID NO: 38 - protein, 120 aa, Mus musculus
EVQLQESGPGLVKPSQSLSLTCSVTGYSITSGYYWNWIRQFPENKLEWMGYIRYDGSNNYNPSLNNRISITRDASKNQFFLKLNSVTTEDTATYYCARGWLLWFYAVDYWGQGTSVTVSS

SEQ ID NO: 39 - protein, 112 aa, Mus musculus
DVVMTQTPLSLPVSLGDQASISCRSSQSIVHTNGNTYLEWYLQKSGQSPKLLIYKVSNRLSGVPDRFSGSGSGTDFTLKISRVEAEDLGIYYCFQGSHVPWTFGGGTKLEIK

SEQ ID NO: 40 - protein, 117 aa, Mus musculus
QVQLKESGPGLVAPSQSLSITCTVSGFSLTSYGVSWVRQPPGKGLEWLGVIWGDGNTNYHSALISRLSISKDNSKSQVFLKLNSLQTDDTATYYCARTNWDGWFAYWGQGTLVTVSA

SEQ ID NO: 41 - protein, 108 aa, Mus musculus
DIVMTQSHKFMSTSVGDRVSITCKASQDVGTAVAWYQQKPGQSPKLLIYWASTRHTGVPDRFTGSGSGTDFTLTISNVQSEDLADYFCQQYRSYPLGTFGGGTKLEIK

SEQ ID NO: 42 - protein, 330 aa, Artificial
  Homo sapiens with substitutions
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 330 aa, Artificial
  Homo sapiens with substitutions
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 330 aa, Artificial
  Homo sapiens with substitutions
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - protein, 330 aa, Artificial
  Homo sapiens with substitutions
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 138 aa, Homo sapiens
TGVHSGKPIPNPLLGLDSTGHLPKPTLWAEPGSVITQGSPVTLRCQGGQETQEYRLYREKKTALWITRIPQELVKKGQFPIPSITWEHAGRYRCYYGSDTAGRSESSDPLELVVTGAGALQSTASLFVVSLSLLHLYS

SEQ ID NO: 47 - protein, 141 aa, Homo sapiens
TGVHSGKPIPNPLLGLDSTYIKPTLSAQPSPVVNSGGNVILQCDSQVAFDGFSLCKEGEDEHPQCLNSQPHARGSSRAIFSVGPVSPSRRWWYRCYAYDSNSPYEWSLPSDLLELLVLGVGALQSTASLFVVSLSLLHLYS

SEQ ID NO: 48 - protein, 139 aa, Homo sapiens
TGVHSGKPIPNPLLGLDSTSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQGALQSTASLFVVSLSLLHLYS

SEQ ID NO: 49 - protein, 348 aa, Homo sapiens
TGVHSGKPIPNPLLGLDSTFYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 50 - protein, 239 aa, Homo sapiens
TGVHSGKPIPNPLLGLDSTGHLPKPTLWAEPGSVITQGSPVTLRCQGGQETQEYRLYREKKTALWITRIPQELVKKGQFPIPSITWEHAGRYRCYYGSDTAGRSESSDPLELVVTGAYIKPTLSAQPSPVVNSGGNVILQCDSQVAFDGFSLCKEGEDEHPQCLNSQPHARGSSRAIFSVGPVSPSRRWWYRCYAYDSNSPYEWSLPSDLLELLVLGVGALQSTASLFVVSLSLLHLYS

SEQ ID NO: 51 - protein, 240 aa, Homo sapiens
TGVHSGKPIPNPLLGLDSTYIKPTLSAQPSPVVNSGGNVILQCDSQVAFDGFSLCKEGEDEHPQCLNSQPHARGSSRAIFSVGPVSPSRRWWYRCYAYDSNSPYEWSLPSDLLELLVLGVSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQGALQSTASLFVVSLSLLHLYS

SEQ ID NO: 52 - protein, 447 aa, Homo sapiens
TGVHSGKPIPNPLLGLDSTSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQFYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 53 - protein, 338 aa, Homo sapiens
TGVHSGKPIPNPLLGLDSTGHLPKPTLWAEPGSVITQGSPVTLRCQGGQETQEYRLYREKKTALWITRIPQELVKKGQFPIPSITWEHAGRYRCYYGSDTAGRSESSDPLELVVTGAYIKPTLSAQPSPVVNSGGNVILQCDSQVAFDGFSLCKEGEDEHPQCLNSQPHARGSSRAIFSVGPVSPSRRWWYRCYAYDSNSPYEWSLPSDLLELLVLGVSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQGALQSTASLFVVSLSLLHLYS

SEQ ID NO: 54 - protein, 548 aa, Homo sapiens
TGVHSGKPIPNPLLGLDSTYIKPTLSAQPSPVVNSGGNVILQCDSQVAFDGFSLCKEGEDEHPQCLNSQPHARGSSRAIFSVGPVSPSRRWWYRCYAYDSNSPYEWSLPSDLLELLVLGVSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQFYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 55 - protein, 98 aa, Homo sapiens
GHLPKPTLWAEPGSVITQGSPVTLRCQGGQETQEYRLYREKKTALWITRIPQELVKKGQFPIPSITWEHAGRYRCYYGSDTAGRSESSDPLELVVTGA

SEQ ID NO: 56 - protein, 137 aa, homo sapiens
FYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSGPEDQPLTPTGSDPQSGLGRH

SEQ ID NO: 57 - DNA, 35 bp, homo sapiens
acaggcgtgcattcggggcacctccccaagcccac

SEQ ID NO: 58 - DNA, 58 bp, Homo sapiens
cgaggtcgggggatcctcaatggtggtgatgatggtggtgccttcccagaccactctg

SEQ ID NO: 59 - protein, 441 aa, Artificial
  synthetic
GHLPKPTLWAEPGSVITQGSPVTLRCQGGQETQEYRLYREKKTALWITRIPQELVKKGQFPIPSITWEHAGRYRCYYGSDTAGRSESSDPLELVVTGAYIKPTLSAQPSPVVNSGGNVILQCDSQVAFDGFSLCKEGEDEHPQCLNSQPHARGSSRAIFSVGPVSPSRRWWYRCYAYDSNSPYEWSLPSDLLELLVLGVSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQFYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSGPEDQPLTPTGSDPQSGLGRHHHHHHH

SEQ ID NO: 60 - protein, 33 aa, Homo sapiens
ACAGGCGTGCATTCGGGGCACCTCCCCAAGCCC

SEQ ID NO: 61 - protein, 36 aa, Homo sapiens
CCGCCCCGACTCTAGACTAGTGGATGGCCAGAGTGG

SEQ ID NO: 62 - protein, 43 aa, Homo sapiens
CCAGAACACAGGATCCGCCGCCACCATGGTGGTCATGGCGCCC

SEQ ID NO: 63 - protein, 36 aa, Homo sapiens
TTTTCTAGGTCTCGAGTCAATCTGAGCTCTTCTTTC

SEQ ID NO: 64 - DNA, 92 bp, Homo sapiens
acaggcgtgcattcgggtaagcctatccctaaccctctcctcggtctcgattctacggggcacctccccaagcccaccctctgggctgagcc

SEQ ID NO: 65 - DNA, 92 bp, Homo sapiens; identical to SEQ ID NO: 64

SEQ ID NO: 66 - DNA, 93 bp, Homo sapiens
acaggcgtgcattcgggtaagcctatccctaaccctctcctcggtctcgattctacggggcccctccccaaacccaccctctgggctgagcca

SEQ ID NO: 67 - DNA, 93 bp, Homo sapiens
acaggcgtgcattcgggtaagcctatccctaaccctctcctcggtctcgattctacggggaccatccccaagcccaccctgtgggctgagcca

SEQ ID NO: 68 - DNA, 92 bp, Homo sapiens
acaggcgtgcattcgggtaagcctatccctaaccctctcctcggtctcgattctacggggcccttccccaaacccaccctctgggctgagcc

SEQ ID NO: 69 - DNA, 93 bp, Homo sapiens; identical to SEQ ID NO: 66

SEQ ID NO: 70 - DNA, 93 bp, Homo sapiens
acaggcgtgcattcgggtaagcctatccctaaccctctcctcggtctcgattctacggaaaacctacccaaacccatcctgtgggccgagcca

SEQ ID NO: 71 - DNA, 92 bp, Homo sapiens; identical to SEQ ID NO: 68

SEQ ID NO: 72 - DNA, 37 bp, Homo sapiens
ccgccccgactctagatcatctctggctgtgctgagc

SEQ ID NO: 73 - DNA, 36 bp, Homo sapiens
ccgccccgactctagactagtggatggccagagtgg

SEQ ID NO: 74 - DNA, 37 bp, Homo sapiens
ccgccccgactctagatcaggcatagacactgggctc

SEQ ID NO: 75 - DNA, 36 bp, Homo sapiens
ccgccccgactctagactagtggatggccagggtgg

SEQ ID NO: 76 - DNA, 37 bp, Homo sapiens
ccgccccgactctagatcaggcgtagatgctgggctc

SEQ ID NO: 77 - DNA, 115 bp, Homo sapiens
cgccccgactctagatcaagagtaaagatgcagaagactaagactgactacaaatagggaagcagtagattgaagagcaccctcaccagccttggagtcggacttgttttgtggt

SEQ ID NO: 78 - DNA, 37 bp, Homo sapiens
ccgccccgactctagatcactccaccactctgaaggg

SEQ ID NO: 79 - DNA, 37 bp, homo sapiens
ccgccccgactctagatcaatcttgggggtttctctg

SEQ ID NO: 80 - protein, 14 aa, artificial
  synthetic
GKPIPNPLLGLDST

SEQ ID NO: 81 - protein, 119 aa, Mus musculus
QVQLQQSGPELVKPGASVKISCKASGYSFTNFYIHWVRQRPGQGLDWIGWIFPGSGETKFNEKFKVKATLTADTSSSTAYMQLNSLTSEDSAVYFCARSWNYDARWGYWGQGTSVTVSS

SEQ ID NO: 82 - protein, 111 aa, Mus musculus
QIVLTQSPASLAVSLGQRATISCRASESIDSYGISFLHWYQQKPGQPPKLLIYRASNLESGIPARFSGSGSRPDFTLTINPVEADDVATYYCQQSNEDPFTFGSGTKLEIK

SEQ ID NO: 83 - protein, 119 aa, Mus musculus
DVQLVESGPELVKPGASVKISCKASGYSFTNYYMQWVKQRPGQGLEWIGWIFPGGGESNYNEKFKGKATLSADTSSTTAYMQLSSLTSEDSAVYFCARTWNYDARWGYWGQGTTVTVSS

SEQ ID NO: 84 - protein, 111 aa, Mus musculus
DIQMTQSPASLTVSLGQRATISCRPSENIDSYGISFMHWYQQKPGQPPKLLIYRASNLESGIPVRFSGSGSRTDFTLTINPVEADDVATYYCQQTNEDPFTFGSGTKLEIK

SEQ ID NO: 85 - protein, 119 aa, Mus musculus
EVQLKQSGPELVKPGASVKISCKASGYSFTNYYIQWVKQRPGQGLEWIGWIFPGNGETNYNEKFKGKATLTADTSSSTAYMQLSSLTSEDSAVYFCARTWNYDARWGYWGQGTSVTVSS

SEQ ID NO: 86 - protein, 111 aa, Mus musculus
DIVLTQSPASLAVSLGQRATISCIPSESIDSYGISFMHWYQQKPGQPPKLLIYRASNLESGIPARFSGSGSRTDFTLTINPVEADDVATYYCQQSNEDPFTFGSGTKLEIK

SEQ ID NO: 87 - protein, 119 aa, Mus musculus
QVQLQQSGPELVKPGASVKISCKASGYSFTNYYMQWVKQRPGQGLEWIGWIFPGSGESNYNEKFKGKATLSADTSSTTAYMQLSSLTSEDSAVYFCARTWNYDARWGYWGQGTSVTVSS

SEQ ID NO: 88 - protein, 111 aa, Mus musculus
DILLTQSPASLTVSLGQRATISCRANESIDSYGISFMHWYQQKPGQPPKLLIYRASNLDSGIPARFSGSGSRTDFTLTINPVEADDVATYYCQQSNEDPFTFGSGTKLEIK

SEQ ID NO: 89 - protein, 119 aa, Mus musculus
EFQLQQSGPELVKPGASVKISCKASGYSFTNYYMQWVKQRPGQGLEWIGWIFPGSGESNYNEKFKGKATLSADTSSTTAYMQLSSLTSEDSAVYFCARTWNYDARWGYWGQGTTLTVSS

SEQ ID NO: 90 - protein, 111 aa, Mus musculus
DIVMTQSPASLAVSLGQRATISCIPSESIDSYGISFMHWYQQKPGQPPKLLIYRASNLESGIPARFSGSGSRTDFTLTINPVEADDVATYYCQQSNEDPFTFGSGTKLEIK

SEQ ID NO: 91 - protein, 119 aa, Mus musculus
EVKLEESGPELVKPGASVKLSCKASGYTFTNYYMQWVKQRPGQGLEWIGWIFPGSGESSYNEKFKGKATLSADTSSTTAYMQLSSLTSEDSAVYFCARTWNYDARWGYWGQGTTLTVSS

SEQ ID NO: 92 - protein, 111 aa, Mus musculus
DILMTQSPASLAVSLGQRATISCIPSESIDSYGISFMHWYQQKPGQPPKLLIYRASNLESGIPARFSGSGSRTDFTLTINPVEADDVATYYCQQSNEDPFTFGSGTKLELK

SEQ ID NO: 93 - protein, 119 aa, Mus musculus
EVKLQQSGPELVKPGASVKISCKASGYSFTNYYIHWVKQRPGQGLEWIGWIFPGSGETNYNEKFKVKATLSADTSSTTAYMQLSSLTSEDSAVYFCARTWNYDARWGYWGQGTTLTVSS

SEQ ID NO: 94 - protein, 111 aa, Mus musculus; identical to SEQ ID NO: 92

SEQ ID NO: 95 - protein, 119 aa, Mus musculus
QVQLKESGPELVKPGASVKISCKTSGYSFTNYYIHWVKQRPGQGLEWIGWIFPGSGDTNYNEKFKGKATLTADTSSNTASMHLSSLTSEDSAVYFCARTWNYDARWGYWGQGTTLTVSS

SEQ ID NO: 96 - protein, 111 aa, Mus musculus
DVVVTQTPASLAVSLGQRATISCRASESIDSYGISFMHWYQQKPGQPPKLLIYRASNLESGIPARFSGSGSRTDFTLTINPVEADDVATYYCQQSNEDPFTFGSGTKLEIK

SEQ ID NO: 97 - protein, 119 aa, Mus musculus
EVQLQQSGPDLVKPGASVKMSCKASGYSFTNFYIHWVKQRPGQGLEWIGWIFPGNGETNYSEKFKGKATLTADTSSSTAYMQFNSLTYEDSAVYFCARTWNYDARWVYWGQGTTVTVSS

SEQ ID NO: 98 - protein, 111 aa, Mus musculus
DIVMTQSPASLAVSLGQRATISCRVSESIDSYGISFMHWYQQKSGQPPKVLIYRASTLESGIPARFSGSGSRTDFTLTINPVEADDVATYYCQQSNEDPFTFGSGTKLEIK

SEQ ID NO: 99 - protein, 119 aa, Mus musculus
EVKLQQSGPDLVKPGASVKISCKASGYSFTNFYIHWVKQRPGQGLEWIGWIFPGNGETNYSEKFKGKATLTADTSSSTAYMQFNSLTYEDSAVYFCARTWNYDARWGYWGQGTTLTVSS

SEQ ID NO: 100 - protein, 111 aa, Mus musculus
EIVLTQSPASLAVSLGQRATISCRASDGIDSYGISFMHWYQQKPGQPPTVLIYRASILESGIPARFSGSGSRTDFTLTINPVEADDVATYYCQQTNEDPFTFGSGTKLEIK

SEQ ID NO: 101 - protein, 119 aa, Mus musculus; identical to SEQ ID NO: 83

SEQ ID NO: 102 - protein, 111 aa, Mus musculus
DILLTQSPASLAVSLGQRATISCRASDGIDSYGISFMHWYQQKPGQPPTLLIYRASTLESGIPARFSGSGSRTDFTLTINPVEADDVATYYCQQTNEAPFTFGSGTKLELK

SEQ ID NO: 103 - protein, 119 aa, Mus musculus
DVQLQESGPELVKPGASVKISCKSSGYSFTNFYIHWVKQRPGQGLDWIGWIFPGTGETNFNEKFKVKAALTADTSSSTVYMQLSTLTSEDSAVYFCARSWNYDARWGYWGQGTSITVSS

SEQ ID NO: 104 - protein, 107 aa, Mus musculus
DVVMTQTPAFLAVSLGQRATISCRASESIDSYMSWYQQKPGQPPKVLIYGASNLESGIPARFSGSGSGTDFTLNIHPVEEEDAATYYCQQSNEDPWTFGGGTKLEIK

SEQ ID NO: 105 - protein, 119 aa, Mus musculus
EVQLQESGPELVKPGASVKISCKASGYSFRNYYIQWVKQRPGQGLEWIGWIFPGNYETNYNEKFKGKATLSADTSSTTAYMQLSSLTSEDSAVYFCARSWNYDARWGYWGQGTSVTVSS

SEQ ID NO: 106 - protein, 111 aa, Mus musculus
ENVLTQSPASLAVSLGQRATISCRASESIDSFGISFMHWYQQKPGQPPKLLIYRASNLESGIPARFSGSGSGPDFSLTIDPVEADDVATYYCQQSNEAPFTFGSGTKLEIK

SEQ ID NO: 107 - protein, 119 aa, Mus musculus
QVQLKQSGPELVKPGASVKISCKASGYSFTNYYIHWVKQRPGQGLEWIGWIFPGSGETNFNEKFKGKATLTADTSSSTAYMQFSSLTSEDSAVYFCARTWNYDARWGYWGQGTTVTVSS

SEQ ID NO: 108 - protein, 111 aa, Mus musculus
EIVLTQSPASLAVSLGQRATISCRASESIDSYGISFMHWYQQKPGQPPKLLIYRASNLESGIPARFSGSGSRTDFTLTINPVEADDVATYYCQQSNEDPFTFGSGTKLEIK

SEQ ID NO: 109 - protein, 119 aa, Mus musculus; identical to SEQ ID NO: 107

SEQ ID NO: 110 - protein, 111 aa, Mus musculus
DILMTQSPASLAVSLGQRATISCRASEGIDSYGISFMHWYQQKPGQPPTLLIYRASNLESGIPARFSGSGSRTDFTLTINPVEADDVATYYCQQTNEDPFTFGSGTKLEIK

SEQ ID NO: 111 - protein, 119 aa, Mus musculus
EVQLQESGPELVKPGASVKISCKTSGYSFTNYYIHWVKQRPGQGLEWIGWIFPGSGETNYNEKFKGKATLSADTSSTTAYMQLSSLTSEDSAVYFCARTWNYDARWGYWGQGTTVTVSS

SEQ ID NO: 112 - protein, 111 aa, Mus musculus
EIQMTQSPASLAVSLGQRATISCRASEGIDSYGISFMHWYQQKPGQPPTLLIYRASNLVSGIPARFSGSGSRTDFTLTINPVEADDVATYYCQQTNEDPFTFGSGTKLEIK

SEQ ID NO: 113 - protein, 119 aa, Mus musculus
DVQLQESGPDLVKPGASVKISCKASGYSFRNYYIQWVKQRPGQGLEWIGWIFPGNNETNYNEKFKGKATLSADTSSTTAYMQLSSLTSEDSAVYFCARSWNYDARWGYWGQGTTLTVSS

SEQ ID NO: 114 - protein, 111 aa, Mus musculus
EILLTQSPASLAVSLGQRATISCRASETIDSYGISFMHWYQQKPGQPPKLLIYRASNLESGIPARFSGSGSRTDFTLTINPVEADDVATYYCQQSNEDPFTFGSGTKLEIK

SEQ ID NO: 115 - protein, 119 aa, Mus musculus
QVQLKESGPELVKPGASVKISCKASGYSFTNYYIHWVKQRPGQGLEWIGWIFPGSGETNYSEKFKGEAILTADTSSNTAYMQLSSLTSEDSAVYFCARSWNYDARWGYWGQGTTLTVSS

SEQ ID NO: 116 - protein, 111 aa, Mus musculus
EIVLTQSPASLAVSLGQRATISCRASEIIDSYGISFMHWYQQKPGQPPKLLIYRASNLESGIPARFSGSGSRTDFTLTINPVEADDVATYYCQQSNEDPFTFGSGTKLEIK

SEQ ID NO: 117 - protein, 119 aa, Mus musculus
QIQLQQSGPELVKPGASVKISCKASGYSFTNYYIQWVKQRPGQGLEWIGWIFPGSGETNYNENFKAKATLSADTSSTTAYMQLSSLTSEDSAVYFCARTWNYDARWGYWGQGTSVTVSS

SEQ ID NO: 118 - protein, 111 aa, Mus musculus
QIVLSQSPVSLAVSPGQRATISCRASESIDSYGISFMHWYKQKPGQPPKLLIYRASNLESGIPARFSGSGSRTDFTLTINPVEADDVATYYCQQSNEDPFTFGSGTKLEIK

SEQ ID NO: 119 - protein, 119 aa, Mus musculus
EVHLQQSGPELVKPGASVKISCKASGYSFTNYYIQWVKQRPGQGLEWIGWIFPGSGETNYNENFRAKATLSADTSSTTAYMQLSSLTSEDSAVYFCARTWNYDARWGYWGQGTTVTVSS

SEQ ID NO: 120 - protein, 112 aa, Mus musculus
EILLTQSPPASLAVSLGQRVTISCRPSENIDSYGISFMHWYQQKPGQPPKLLIYRASNLESGIPVRFSGSGSRTDFTLTINPVEADDVATYYCQQSNEDPFTFGSGTKLEIK

SEQ ID NO: 121 - protein, 119 aa, Mus musculus
QVQLKESGPELVKPGASVKISCKTSGYIFTNYYIHWVKQRPGQGLEWIGWIFPGSGDTNYNEKFKGKATLTADTSSSTASMQLSSLTSEDSAVYFCARTWNYDARWGYWGQGTTVTVSS

SEQ ID NO: 122 - protein, 111 aa, Mus musculus
DILLTQSPASLAVSLGQRATISCRPSENIDSYGISFMHWCQQKPGQPPKLLIYRASNLESGIPVRFSGSGSRTDFTLTINPVEADDVATYYCQQSNEDPFTFGSGTKLEIK

SEQ ID NO: 123 - protein, 119 aa, Mus musculus
QVQLKESGPELVKPGASVKISCKASGYSFTNYYIHWVKQRPGQGLEWIGWIFLGSGETNYNEKFKGEAILTADTSSTTAYMQLSSLTSEDSAVYFCARSWNYDARWGYWGQGTTLTVSS

SEQ ID NO: 124 - protein, 111 aa, Mus musculus
DILLTQSPASLAVSLGQRATISCRVSESIDSYGISFMHWYQQKSGQPPKVLIYRASTLESGIPARFSGSGSRTDFTLTINPVEADDVATYYCQQSNEDPFTFGSGTKLEIK

SEQ ID NO: 125 - protein, 119 aa, Mus musculus
EVQLQQSGPELVKPGASVKISCKASGYSFTNFYIHWVKQRPGQGLDWIGWIFPGSGETNYNERFKGKATLTSDTSSSTAYMQLSSLTSEDSAVYFCARSWNYDARWGYWGQGTTLTVSS

SEQ ID NO: 126 - protein, 111 aa, Mus musculus
EIVLTQSPASLAVSLGQRATISCRASESIDSYGISFIHWYQQKPGQPPKLLIYRASNLESGIPARFSGSGSRTDFTLTINPVEAEDVATYYCQQSNEDPFTFGSGTKLEIK

SEQ ID NO: 127 - protein, 119 aa, Mus musculus
EVQLQQSGPELVKPGASVKISCKSSGYSFTNFYIHWVKQRPGQGLDWIGWIFPGSGETNFNEKFKVKAALTADTSSNTAYMQLSSLTSEDSAVYFCARSWNYDARWGYWGQGTTVTVSS

SEQ ID NO: 128 - protein, 111 aa, Mus musculus
QIVLTQTPASLAVSLGQRATISCRASESIDSYGISFMHWYQQKPGQPPKLLIYRASNLESGIPARFSGSGSRPDFTLTINPVEADDVATYYCQQSNEDPFTFGSGTKLEIK

SEQ ID NO: 129 - protein, 118 aa, Mus musculus
QVQLKESGPGLVAPSQSLSITCTVSGFSLTSYGVSWVRQPPGKGLEWLGIIWGDGSTNYHSALVSRLSISKDNSKSQVFLKLNSLQTDDTATYYCAKPNWDYYAMDYWGQGTSVTVSS

SEQ ID NO: 130 - protein, 111 aa, Mus musculus
DAVMTQTPASLAVSLGQRATISCRASESVDSYGISFMHWYQQKPGQPPKLLIYRASILESGIPARFSGSGSRPDFSLTINPVEADDVATYYCQQSNEDPFTFGSGTKLEIK

SEQ ID NO: 131 - protein, 119 aa, Mus musculus; identical to SEQ ID NO: 103

SEQ ID NO: 132 - protein, 107 aa, Mus musculus
DIQMTQTTSSLSASLGDRVTISCRASQDISNYLNWYQQKVDGTVKLLISYTSRLHSGVPSRFSGSGSGTDYSLTISNLEQEDIATYFCQQGITLPLTFGAGTKLELK

SEQ ID NO: 133 - protein, 119 aa, Mus musculus; identical to SEQ ID NO: 99

SEQ ID NO: 134 - protein, 111 aa, Mus musculus
DVVMTQTPASLAVSLGQRATISCRASDGIDSYGISFMRWYQQKPGQPPTLLIYRASTLESGIPARFSGSGSRTNFTLTINPVEADDVATYYCQQTNEDPFTFGSGTKLEIK

SEQ ID NO: 135 - protein, 119 aa, Mus musculus
QRELQQSGPELVKPGASVNISCKASGYSFTNHYINWVKQRPGQGLEWIGWIFPGNGDTNYNEKFKGKATLTADTSSSTAYMQLSSLTSEDSAVYFCARTWNYDARWGYWGQGTTVTVSS

SEQ ID NO: 136 - protein, 111 aa, Mus musculus
DVVMTQTPAFLAVSLGQRATISCRASESIDSYGISFMHWYQQKPGQPPKVLIYRTSNLESGIPARFSGSGSRTDFTLTINPVEADDVATYYCQQSNEDPFTFGSGTKLEIK

SEQ ID NO: 137 - protein, 119 aa, Mus musculus
QVQLKESGPELVKPGTSVKISCKASGYNFRNYYIQWVKQRPGQGLEWIGWIFPGNNETNYNEKFKGKATLSADTSSTTAYMQLSSLTSEDSAVYFCARSWNYDARWGYWGQGTTVTVSS

SEQ ID NO: 138 - protein, 111 aa, Mus musculus
DVVMTQTPASLAVSLGQRATISCRASEIIDNYGISFIHWYQQKPGQPPKLLIYRASNLESGIPARFSGSGSRTDSTLTINPVGADDVATYYCQQSNEDPFTFGSGTKLELK

SEQ ID NO: 139 - protein, 5 aa, Mus musculus
NYYMQ

SEQ ID NO: 140 - protein, 17 aa, Mus musculus
WIFPGSGESSYNEKFKG

SEQ ID NO: 141 - protein, 10 aa, Mus musculus
TWNYDARWGY

SEQ ID NO: 142 - protein, 15 aa, Mus musculus
IPSESIDSYGISFMH

SEQ ID NO: 143 - protein, 7 aa, Mus musculus
RASNLES

SEQ ID NO: 144 - protein, 9 aa, Mus musculus
QQSNEDPFT

SEQ ID NO: 145 - protein, 5 aa, Mus musculus
NFYIH

SEQ ID NO: 146 - protein, 17 aa, Mus musculus
WIFPGSGETKFNEKFKV

SEQ ID NO: 147 - protein, 10 aa, Mus musculus
SWNYDARWGY

SEQ ID NO: 148 - protein, 15 aa, Mus musculus
RASESIDSYGISFLH

SEQ ID NO: 149 - protein, 7 aa, Mus musculus
RASNLES

SEQ ID NO: 150 - protein, 9 aa, Mus musculus
QQSNEDPFT

SEQ ID NO: 151 - protein, 5 aa, Mus musculus
NYYVQ

SEQ ID NO: 152 - protein, 17 aa, Mus musculus
WIFPGSGETNYNEKFKA

SEQ ID NO: 153 - protein, 9 aa, Mus musculus
QQTNEDPFT

SEQ ID NO: 154 - protein, 5 aa, Mus musculus
NYYMQ

SEQ ID NO: 155 - protein, 17 aa, Mus musculus
WIFPGGGESNYNEKFKG

SEQ ID NO: 156 - protein, 15 aa, Mus musculus
IPSESIDSYGISFMH

SEQ ID NO: 157 - protein, 5 aa, Mus musculus
NYYIQ

SEQ ID NO: 158 - protein, 17 aa, Mus musculus
WIFPGNGETNYNEKFKG

SEQ ID NO: 159 - protein, 15 aa, Mus musculus
RANESIDSYGISFMH

SEQ ID NO: 160 - protein, 7 aa, Mus musculus
RASNLDS

SEQ ID NO: 161 - protein, 17 aa, Mus musculus
WIFPGSGESNYNEKFKG

SEQ ID NO: 162 - protein, 15 aa, Mus musculus
IPSESIDSYGISFMH

SEQ ID NO: 163 - protein, 5 aa, Mus musculus
NYYIH

SEQ ID NO: 164 - protein, 17 aa, Mus musculus
WIFPGSGETNYNEKFKV

SEQ ID NO: 165 - protein, 15 aa, Mus musculus
RASESIDSYGISFMH

SEQ ID NO: 166 - protein, 17 aa, Mus musculus
WIFPGSGDTNYNEKFKG

SEQ ID NO: 167 - protein, 15 aa, Mus musculus
RVSESIDSYGISFMH

SEQ ID NO: 168 - protein, 7 aa, Mus musculus
RASTLES

SEQ ID NO: 169 - protein, 17 aa, Mus musculus
WIFPGNGETNYSEKFKG

SEQ ID NO: 170 - protein, 10 aa, Mus musculus
TWNYDARWVY

SEQ ID NO: 171 - protein, 15 aa, Mus musculus
RASDGIDSYGISFMH

SEQ ID NO: 172 - protein, 7 aa, Mus musculus
RASILES

SEQ ID NO: 173 - protein, 17 aa, Mus musculus
WIFPGNGETNYSEKFKG

SEQ ID NO: 174 - protein, 15 aa, Mus musculus
RASDGIDSYGISFMH

SEQ ID NO: 175 - protein, 9 aa, Mus musculus
QQTNEAPFT

SEQ ID NO: 176 - protein, 5 aa, Mus musculus
NYYIN

SEQ ID NO: 177 - protein, 17 aa, Mus musculus
WIFPGNGDTNYNEKFKG

SEQ ID NO: 178 - protein, 11 aa, Mus musculus
RASESIDSYMS

SEQ ID NO: 179 - protein, 7 aa, Mus musculus
GASNLES

SEQ ID NO: 180 - protein, 9 aa, Mus musculus
QQSNEDPWT

SEQ ID NO: 181 - protein, 15 aa, Mus musculus
RPSENIDSYGISFMH

SEQ ID NO: 182 - protein, 17 aa, Mus musculus
WIFPGTGETNFNEKFKV

SEQ ID NO: 183 - protein, 10 aa, Mus musculus
SWNYDARWGY

SEQ ID NO: 184 - protein, 15 aa, Mus musculus
RASESIDSFGISFMH

SEQ ID NO: 185 - protein, 9 aa, Mus musculus
QQSNEAPFT

SEQ ID NO: 186 - protein, 17 aa, Mus musculus
WIFPGSGETNFNEKFKG

SEQ ID NO: 187 - protein, 15 aa, Mus musculus
RASESIDSYGISFMH

SEQ ID NO: 188 - protein, 17 aa, Mus musculus
WIFPGSGETNFNEKFKG

SEQ ID NO: 189 - protein, 15 aa, Mus musculus
RASESIDSYGISFMH

SEQ ID NO: 190 - protein, 7 aa, Mus musculus
RASNLVS

SEQ ID NO: 191 - protein, 17 aa, Mus musculus
WIFPGSGETNYNEKFKG

SEQ ID NO: 192 - protein, 15 aa, Mus musculus
RASETIDSYGISFMH

SEQ ID NO: 193 - protein, 17 aa, Mus musculus
WIFPGNNETNYNEKFKG

SEQ ID NO: 194 - protein, 15 aa, Mus musculus
RASEIIDSYGISFMH

SEQ ID NO: 195 - protein, 17 aa, Mus musculus
WIFPGSGETNYSEKFKG

SEQ ID NO: 196 - protein, 15 aa, Mus musculus
RASESIDSYGISFMH

SEQ ID NO: 197 - protein, 17 aa, Mus musculus
WIFPGSGETNYNENFKA

SEQ ID NO: 198 - protein, 17 aa, Mus musculus
WIFPGSGETNYNENFRA

SEQ ID NO: 199 - protein, 15 aa, Mus musculus
RPSENIDSYGISFMH

SEQ ID NO: 200 - protein, 17 aa, Mus musculus
WIFPGSGDTNYNEKFKG

SEQ ID NO: 201 - protein, 15 aa, Mus musculus
RVSESIDSYGISFMH

SEQ ID NO: 202 - protein, 17 aa, Mus musculus
WIFLGSGETNYNEKFKG

SEQ ID NO: 203 - protein, 15 aa, Mus musculus
RASESIDSYGISFIH

SEQ ID NO: 204 - protein, 17 aa, Mus musculus
WIFPGSGETNYNERFKG

SEQ ID NO: 205 - protein, 15 aa, Mus musculus
RASESIDSYGISFMH

SEQ ID NO: 206 - protein, 17 aa, Mus musculus
WIFPGSGETNFNEKFKV

SEQ ID NO: 207 - protein, 15 aa, Mus musculus
RASESVDSYGISFMH

SEQ ID NO: 208 - protein, 5 aa, Mus musculus
SYGVS

SEQ ID NO: 209 - protein, 16 aa, Mus musculus
IIWGDGSTNYHSALVS

SEQ ID NO: 210 - protein, 10 aa, Mus musculus
PNWDYYAMDY

SEQ ID NO: 211 - protein, 11 aa, Mus musculus
RASQDISNYLN

SEQ ID NO: 212 - protein, 7 aa, Mus musculus
YTSRLHS

SEQ ID NO: 213 - protein, 9 aa, Mus musculus
QQGITLPLT

SEQ ID NO: 214 - protein, 449 aa, Artificial
  Chimeric human murine
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 215 - protein, 214 aa, artificial
  chimeric human murine
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC